(19) United States
(12) Patent Application Publication
(10) Pub. No.: US 2023/0364251 A1
(43) Pub. Date: Nov. 16, 2023

(54) METHODS FOR THE SYNTHESIS OF PROTEIN-DRUG CONJUGATES

(71) Applicant: Cidara Therapeutics, Inc., San Diego, CA (US)

(72) Inventors: Allen BORCHARDT, San Diego, CA (US); Robert Michael HUGHES, San Diego, CA (US)

(21) Appl. No.: 18/019,944

(22) PCT Filed: Aug. 6, 2021
(86) PCT No.: PCT/US2021/045074

§ 371 Date: Feb. 6, 2023

Related U.S. Application Data
(60) Provisional application No. 63/062,377, filed on Aug. 6, 2020.
(60) Provisional application No. 63/154,514, filed on Feb. 26, 2021.

(30) Foreign Application Priority Data
Sep. 9, 2020 (WO) PCT/US2020/050022

(51) Int. Cl. A61K 47/68

(52) U.S. Cl. CPC A61K 47/6803

(57) ABSTRACT

The present invention relates to intermediates and methods for synthesizing protein-drug conjugates that can be used for the treatment of diseases and related conditions.

### SEQUENCE LISTING

The instant application contains a Sequence Listing which has been submitted electronically in ASCII format and is hereby incorporated by reference in its entirety. Said ASCII copy, created on Aug. 4, 2021, is named 50945-068WO2_Sequence_Listing_08_04_21_ST25 and is 12,515 bytes in size.

### BACKGROUND

This disclosure features methods for the synthesis of conjugates useful for the treatment of diseases and conditions related thereto.

The utility of many therapeutics, such as small molecule therapeutic agents and biologics such as peptides, polypeptides, and polynucleotides, suffer from inadequate serum half-lives. This necessitates the administration of such therapeutics at high frequencies and/or higher doses, or the use of sustained release formulations in order to maintain the serum levels necessary for therapeutic effects. Frequent systemic administration of drugs is associated with considerable negative side effects. For example, frequent systemic injections represent a considerable discomfort to the subject, pose a high risk of administration related infections, and may require hospitalization or frequent visits to the hospital, in particular when the therapeutic is to be administered intravenously. Moreover, in long term treatments, daily intravenous injections can also lead to considerable side effects of tissue scarring and vascular pathologies caused by the repeated puncturing of vessels. Similar problems are known for all frequent systemic administrations of therapeutics. All these factors lead to a decrease in patient compliance and increased cost for the health system. An effective way of increasing therapeutic half-life and efficacy includes conjugating therapeutics (e.g., small molecule therapeutic agents and biologics such as peptides, polypeptides, and polynucleotides) to polypeptides to form, e.g., protein-drug conjugates.

Accordingly, there is a need for convenient synthetic methods that permit the commercial scale production of such protein-drug conjugates. These approaches can be useful alternatives to existing synthetic methods and can achieve higher yield, higher purity, elimination of impurity (e.g., mutagenic impurity), reduced waste stream, or any combination of the above.

### SUMMARY

The disclosure relates to methods and intermediates for making protein-drug conjugates that can be used for the treatment of diseases and related conditions.

In an aspect, the disclosure features a method of synthesizing a conjugate of formula (M-I):

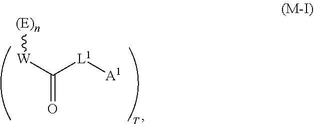

or a pharmaceutically acceptable salt thereof, where n is 1 or 2; W is O, S, NR^{N}, or

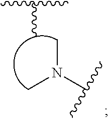

R^{N} is H, optionally substituted C₁-C₂₀ alkyl, or optionally substituted C₁-C₂₀ heteroalkyl;

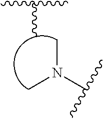

is optionally substituted C₂-C₁₀ heterocyclylene; each E is a polypeptide or polymer; L¹ is a linker including one or more of optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀alkynylene, optionally substituted C₂-C₂₀ heteroalkynylene, optionally substituted C₃-C₂₀ carbocyclylene, optionally substituted C₂-C₂₀ heterocyclylene, optionally substituted C₆-C₂₂ arylene, optionally substituted C₂-C₂₀ heteroarylene, carbonyl, thiocarbonyl, sulfonyl, phosphoryl, optionally substituted amino, O, and S; each A¹ is a therapeutic agent, or each A¹ is, independently, selected from any one of H, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, and optionally substituted amino; T is an integer from 1 to 20; and each squiggly line in formula (M-I) indicates that

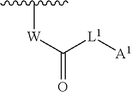

is covalently attached to each E, said method including:
(a) providing a first composition including E;
(b) providing a second composition including a compound of formula (F-I) or salt thereof:

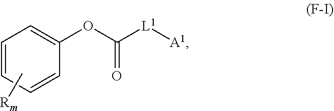

where m is 0, 1, 2, 3, or 4, and each R is, independently, halo, cyano, nitro, optionally substituted C₁-C₆ alkyl group, or optionally substituted C₁-C₆ heteroalkyl group; and
(c) combining the first composition, the second composition, and a buffer to form a mixture.

In some embodiments, E is a polypeptide.

In some embodiments, E is an Fc domain monomer, an Fc domain, an Fc-binding peptide, an albumin protein, or an albumin protein-binding peptide.

In some embodiments, E is an Fc domain monomer, an Fc domain, or an Fc-binding peptide. In some embodiments, E is an Fc domain monomer or an Fc domain.

In some embodiments, E includes at least one lysine residue. In some embodiments, the squiggly line in formula (M-I) is covalently bound to a lysine residue of each E. In some embodiments, W is NR^{N}. In some embodiments, R^{N} is H or optionally substituted C₁-C₂₀ alkyl. In some embodiments, R^{N} is H.

In some embodiments, E includes at least one cysteine residue. In some embodiments, the squiggly line in formula (M-I) is covalently bound to a cysteine residue of each E. In some embodiments, W is S.

In some embodiments, E includes at least one proline residue. In some embodiments, the squiggly line in formula (M-I) is covalently bound to a proline residue of each E. In some embodiments, is

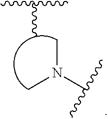

In some embodiments,

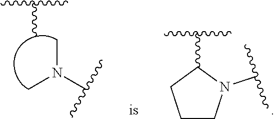

In some embodiments, n is 1. In some embodiments, n is 2.

In some embodiments, E is a polymer.

In some embodiments, E is a polymer derived from one or more species of monomers. In some embodiments, E is a polymer derived from one species of monomer.

In some embodiments, each monomer is, independently, optionally substituted C₁-C₂₀ alkylene (e.g., subunit derived from or including acrylamide), optionally substituted C₁-C₂₀ heteroalkylene (e.g., subunit derived from or including ethylene oxide), optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ carbocyclylene, optionally substituted C₂-C₂₀ heterocyclylene (e.g., saccharide, i.e., carbohydrate (e.g., subunit derived from or including glucose)), optionally substituted C₆-C₂₂ arylene, and optionally substituted C₂-C₂₀ heteroarylene.

In some embodiments, E includes an amine (e.g., NR^{N}R^{N}, where R^{N} is H, optionally substituted C₁-C₂₀ alkyl, or optionally substituted C₁-C₂₀ heteroalkyl), thiol, or hydroxyl. In some embodiments, E includes an amine (e.g., NR^{N}R^{N}, where R^{N} is H, optionally substituted C₁-C₂₀ alkyl, or optionally substituted C₁-C₂₀ heteroalkyl). In some embodiments, E includes —NH₂.

In some embodiments, W is NH.

In some embodiments, A¹ is a therapeutic agent.

In some embodiments, A¹ includes a small molecule. In some embodiments, A¹ includes a monomer, e.g., of a small molecule. In some embodiments, A¹ includes a dimer, e.g., of small molecules. In some embodiments, A¹ includes a monomer or dimer by way of a linker. In some embodiments, A¹ includes a monomer by way of a linker. In some embodiments, A¹ includes a dimer by way of a linker.

In some embodiments, A¹ is a small molecule. In some embodiments, A¹ is a monomer, e.g., of a small molecule. In some embodiments, A¹ is a dimer, e.g., of small molecules. In some embodiments, A¹ is a monomer or dimer by way of a linker. In some embodiments, A¹ is a monomer by way of a linker. In some embodiments, A¹ is a dimer by way of a linker.

In some embodiments, L¹ is:

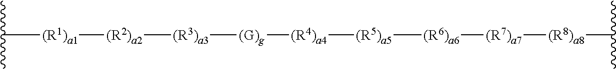

where g is 0 or 1; each of a1, a2, a3, a4, a5, a6, a7, and a8 is, independently, 0 or 1; G is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted C₃-C₁₀ carbocyclylene, optionally substituted C₂-C₁₀ heterocyclylene, optionally substituted C₆-C₁₀ arylene, or optionally substituted C₂-C₁₀ heteroarylene; R¹ is optionally substituted C₁-C₂₀ alkylene, optionally substituted C₁-C₂₀ heteroalkylene, optionally substituted amino, O, or S; R² is optionally substituted C₁-C₂₀ heteroalkylene, optionally substituted C₂-C₂₀ alkenylene, optionally substituted C₂-C₂₀ heteroalkenylene, optionally substituted C₃-C₂₀ cycloalkylene, optionally substituted C₃-C₂₀ heterocycloalkylene, optionally substituted C₆-C₂₂ arylene, or optionally substituted C₂-C₂₀ heteroarylene; R³ is optionally substituted C₁-C₂₀ alkylene, optionally substituted C₁-C₂₀ heteroalkylene, or carbonyl; R⁴ is optionally substituted C₁-C₂₀ alkylene, optionally substituted C₁-C₂₀ heteroalkylene, or carbonyl; R⁵ is optionally substituted C₁-C₂₀ heteroalkylene, optionally substituted C₂-C₂₀ alkenylene, optionally substituted C₂-C₂₀ heteroalkenylene, optionally substituted C₃-C₂₀ cycloalkylene, optionally substituted C₃-C₂₀ heterocycloalkylene, optionally substituted C₆-C₁₈ arylene, optionally substituted C₂-C₂₀ heteroarylene, optionally substituted amino, O, or S; R⁶ is optionally substituted C₁-C₂₀ alkylene, optionally substituted C₁-C₂₀ heteroalkylene, or carbonyl; R⁷ is optionally substituted C₁-C₂₀ heteroalkylene, optionally substituted C₂-C₂₀ alkenylene, optionally substituted C₂-C₂₀ heteroalkenylene, optionally substituted C₃-C₂₀ cycloalkylene, optionally substituted C₃-C₂₀ heterocycloalkylene, optionally substituted C₆-C₁₈ arylene, optionally substituted C₂-C₂₀ heteroarylene, optionally substituted amino, O, or S; and R⁸ is optionally substituted C₁-C₂₀ alkylene, optionally substituted C₁-C₂₀ heteroalkylene, or carbonyl.

In some embodiments, g is 0. In some embodiments, g is 1.

In some embodiments, a1 is 0. In some embodiments, a1 is 1. In some embodiments, a2 is 0. In some embodiments, a2 is 1. In some embodiments, a3 is 0. In some embodiments, a3 is 1. In some embodiments, a4 is 0. In some embodiments, a4 is 1. In some embodiments, a5 is 0. In some embodiments, a5 is 1. In some embodiments, a6 is 0. In some embodiments, a6 is 1. In some embodiments, a7 is 0. In some embodiments, a7 is 1. In some embodiments, a8 is 0. In some embodiments, a8 is 1.

In some embodiments, R¹ is optionally substituted C₁-C₂₀ alkylene or optionally substituted C₁-C₂₀ heteroalkylene. In some embodiments, R¹ is optionally substituted C₁-C₂₀ heteroalkylene. In some embodiments, R¹ is C₁-C₂₀ heteroalkylene.

In some embodiments, R¹ is:

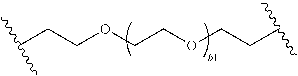

where b1 is 0, 1, 2, 3, 4, 5, 6, 7, or 8.

In some embodiments, R³ is optionally substituted C₁-C₂₀ alkylene or optionally substituted C₁-C₂₀ heteroalkylene. In some embodiments, R³ is optionally substituted C₁-C₂₀ heteroalkylene. In some embodiments, R³ is C₁-C₂₀ heteroalkylene.

In some embodiments, R³ is:

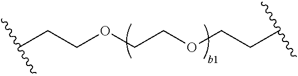

where b1 is 0, 1, 2, 3, 4, 5, 6, 7, or 8.

In some embodiments, R⁴ is optionally substituted C₁-C₂₀ alkylene or optionally substituted C₁-C₂₀ heteroalkylene.

In some embodiments, R⁴ is:

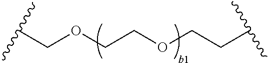

where b1 is 0, 1, 2, 3, 4, 5, 6, 7, or 8.

In some embodiments, R⁵ is optionally substituted amino or optionally substituted C₃-C₂₀ heterocycloalkylene.

In some embodiments, R⁶ is optionally substituted C₁-C₂₀ alkylene.

In some embodiments, R⁷ is optionally substituted amino.

In some embodiments, R⁸ is carbonyl.

In some embodiments, each R is, independently, halo, cyano, nitro, haloalkyl, or

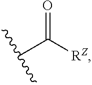

where R^{z} is optionally substituted C₁-C₅ alkyl group or optionally substituted C₁-C₅ heteroalkyl group.

In some embodiments, each R is, independently, halo, cyano, nitro, or haloalkyl.

In some embodiments, each R is, independently, F, Cl, Br, or I.

In some embodiments, each R is F.

In some embodiments, m is 1, 2, 3, 4, or 5. In some embodiments, m is 3, 4, or 5. In some embodiments, m is 3 or 4. In some embodiments, m is 3. In some embodiments, m is 4.

In some embodiments,

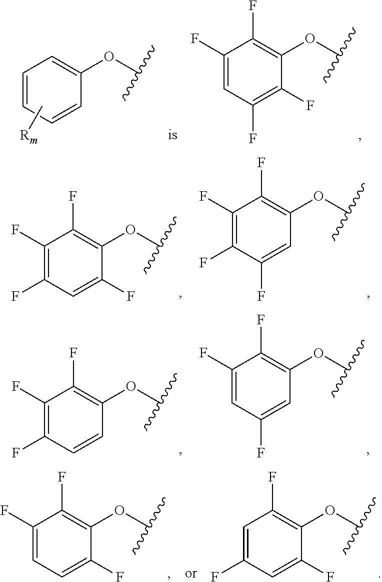

In some embodiments,

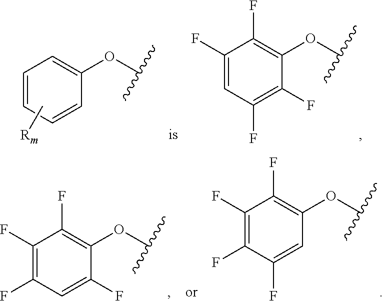

In some embodiments,

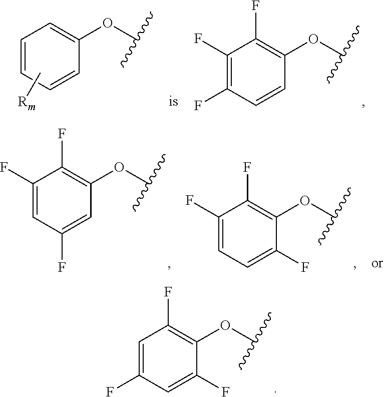

In some embodiments,

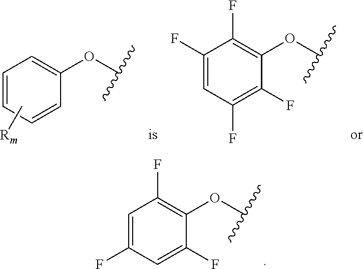

In some embodiments,

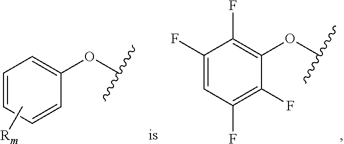

In some embodiments,

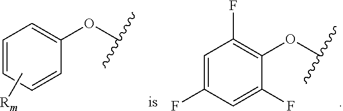

In some embodiments, the compound of formula (F-I) is described by formula (F-I-A):

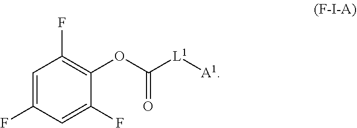

In some embodiments, the compound of formula (F-I) is described by formula (F-I-B):

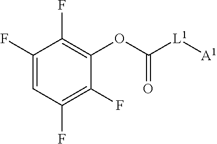

In some embodiments, a compound of formula (F-I), where each R is halo (e.g., F), provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In some embodiments, a compound of formula (F-I), where m is 3, provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In some embodiments, a compound of formula (F-I), where m is 3 and each R is halo (e.g., F), provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In some embodiments, the buffer includes borate or carbonate. In some embodiments, the buffer includes borate. In some embodiments, the buffer includes carbonate.

In some embodiments, the buffer has a pH of about 7.0 to 10.0 (e.g., about 7.0 to 7.5, 7.5 to 8.0, 8.0 to 8.5, 8.5 to 9.0, 9.0 to 9.5, 9.5 to 10.0, 7.0 to 8.0, 7.5 to 8.5, 8.0 to 9.0, 8.5 to 9.5, 9.0 to 10.0, 7.0 to 9.0, 7.5 to 9.5, or 8.0 to 10.0).

In some embodiments, the buffer has a pH of about 7.0. In some embodiments, the buffer has a pH of about 7.1. In some embodiments, the buffer has a pH of about 7.2. In some embodiments, the buffer has a pH of about 7.3. In some embodiments, the buffer has a pH of about 7.4. In some embodiments, the buffer has a pH of about 7.5. In some embodiments, the buffer has a pH of about 7.6. In some embodiments, the buffer has a pH of about 7.7. In some embodiments, the buffer has a pH of about 7.8. In some embodiments, the buffer has a pH of about 7.9. In some embodiments, the buffer has a pH of about 8.0. In some embodiments, the buffer has a pH of about 8.1. In some embodiments, the buffer has a pH of about 8.2. In some embodiments, the buffer has a pH of about 8.3. In some embodiments, the buffer has a pH of about 8.4. In some embodiments, the buffer has a pH of about 8.5. In some embodiments, the buffer has a pH of about 8.6. In some embodiments, the buffer has a pH of about 8.7. In some embodiments, the buffer has a pH of about 8.8. In some embodiments, the buffer has a pH of about 8.9. In some embodiments, the buffer has a pH of about 9.0. In some embodiments, the buffer has a pH of about 9.5. In some embodiments, the buffer has a pH of about 9.6. In some embodiments, the buffer has a pH of about 9.7. In some embodiments, the buffer has a pH of about 9.8. In some embodiments, the buffer has a pH of about 9.9. In some embodiments, the buffer has a pH of about 10.0.

In some embodiments, step (c) is conducted at a temperature of 5 to 50° C., such as 20 to 30° C. (e.g., 20 to 25, 21 to 26, 22 to 27, 23 to 28, 24 to 29, or 25 to 30° C.).

In some embodiments, step (c) is conducted at a temperature of about 25° C.

In some embodiments, step (c) is conducted for about 1 to 24 hours, such as 1 to 12 hours (e.g., 1 to 2, 1 to 5, 2 to 3, 2 to 5, 2 to 10, 2 to 12, 3 to 4, 4 to 5, 1 to 3, 2 to 4, or 3 to 5 hours).

In some embodiments, step (c) is conducted for about 2 hours. In some embodiments, step (c) is conducted for about 3 hours. In some embodiments, step (c) is conducted for about 4 hours. In some embodiments, step (c) is conducted for about 5 hours. In some embodiments, step (c) is conducted for about 6 hours. In some embodiments, step (c) is conducted for about 7 hours. In some embodiments, step (c) is conducted for about 8 hours. In some embodiments, step (c) is conducted for about 9 hours. In some embodiments, step (c) is conducted for about 10 hours. In some embodiments, step (c) is conducted for about 11 hours. In some embodiments, step (c) is conducted for about 12 hours.

In some embodiments, the first composition includes phosphate-buffered saline buffer.

In some embodiments, the buffer has a pH of about 7.0 to 8.0 (e.g., about 7.0 to 7.5, 7.5 to 8.0, 7.0 to 7.2, 7.2 to 7.4, 7.4 to 7.6, 7.6 to 7.8, or 7.8 to 8.0).

In some embodiments, the buffer has a pH of about 7.5.

In some embodiments, the second composition includes DMF (dimethylformamide).

In some embodiments, the method further includes a purification step. In some embodiments, the purification step includes dialysis, e.g., in arginine buffer. In some embodiments, the purification step includes a buffer exchange.

In another aspect, the disclosure features a method of synthesizing a conjugate of formula (M-II):

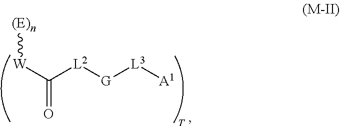

or a pharmaceutically acceptable salt thereof, where n is 1 or 2; W is O, S, NR^{N}, or

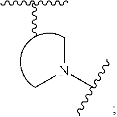

R^{N} is H, optionally substituted C₁-C₂₀ alkylene, or optionally substituted C₁-C₂₀ heteroalkylene;

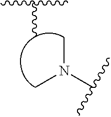

is optionally substituted C₂-C₁₀ heterocyclylene; each E is a polypeptide or polymer; L² is a linker including one or more of optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ carbocyclylene, optionally substituted C₂-C₂₀ heterocyclylene, optionally substituted C₆-C₂₂ arylene, optionally substituted C₂-C₂₀ heteroarylene, carbonyl, thiocarbonyl, sulfonyl, phosphoryl, optionally substituted amino, O, and S; L³ is a linker including one or more of optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ carbocyclylene, optionally substituted C₂-C₂₀ heterocyclylene, optionally substituted C₆-C₂₂ arylene, optionally substituted C₂-C₂₀ heteroarylene, carbonyl, thiocarbonyl, sulfonyl, phosphoryl, optionally substituted amino, O, and S; G is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted C₃-C₁₀ carbocyclylene, optionally substituted C₂-C₁₀ heterocyclylene, optionally substituted C₆-C₁₀ arylene, or optionally substituted C₂-C₁₀ heteroarylene; each A¹ is a therapeutic agent, or each A¹ is, independently, selected from any one of H, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, and optionally substituted amino; T is an integer from 1 to 20; and each squiggly line in formula (M-II) indicates that

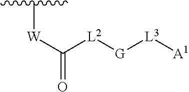

is covalently attached to each E, said method including:
(a) providing a first composition including E;
(b) providing a second composition including a compound of formula (F-II) or salt thereof:

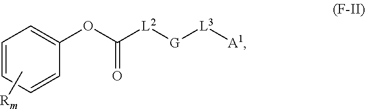

where m is 0, 1, 2, 3, or 4, and each R is, independently, halo, cyano, nitro, optionally substituted C₁-C₆ alkyl group, or optionally substituted C₁-C₆ heteroalkyl group; and
(c) combining the first composition, the second composition, and a buffer to form a mixture.

In some embodiments, G is optionally substituted C₁-C₆ heteroalkylene or optionally substituted C₂-C₁₀ heteroarylene. In some embodiments, G is optionally substituted C₁-C₆ heteroalkylene.

In some embodiments, G is

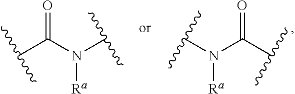

where R^{a} is H, optionally substituted C₁-C₂₀alkylene, or optionally substituted C₁-C₂₀ heteroalkylene.

In some embodiments, G is optionally substituted C₂-C₁₀ heteroarylene. In some embodiments, G is optionally substituted C₂-C₅ heteroarylene. In some embodiments, G is a 5-membered or 6-membered optionally substituted C₂-C₅ heteroarylene. In some embodiments, G is a triazolylene.

In some embodiments, the conjugate of formula (M-II) has the structure of formula (M-II-A):

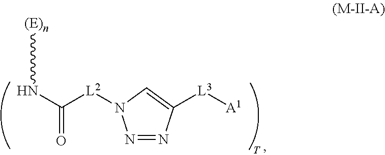

and said method includes:
(a) providing a first composition including E;
(b) providing a second composition including a compound of formula (F-II-A) or salt thereof:

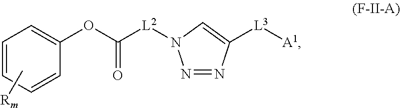

and
(c) combining the first composition, the second composition, and a buffer to form a mixture.

In some embodiments, the synthesis of compound of formula (F-II-A) includes:
(d) providing a third composition including formula (G1-A) or salt thereof:

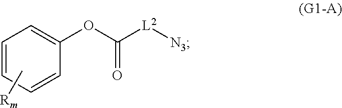

(e) providing a fourth composition including formula (G1-B) or salt thereof:

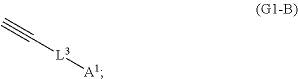

and
(f) combing the third composition and the fourth composition to form a mixture.

In some embodiments, the conjugate of formula (M-II) has the structure of formula (M-II-B):

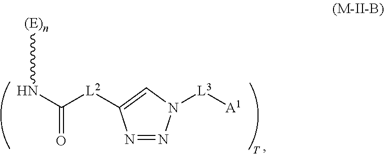

and said method includes:
(a) providing a first composition including E;
(b) providing a second composition including a compound of formula (F-II-A) or salt thereof:

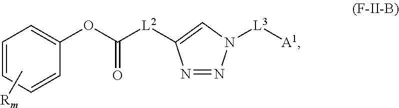

and
(c) combining the first composition, the second composition, and a buffer to form a mixture.

In some embodiments, the synthesis of compound of formula (F-II-B) includes:
(d) providing a third composition including formula (G2-A) or salt thereof:

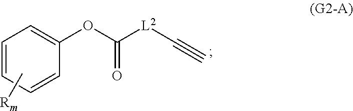

(e) providing a fourth composition including formula (G2-B) or salt thereof:

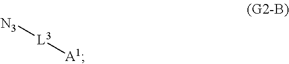

and
(f) combing the third composition and the fourth composition to form a mixture.

In some embodiments, step (f) includes the use of a Cu(I) source.

In some embodiments, the compound of formula (F-II-A) is described by formula (F-II-A-1):

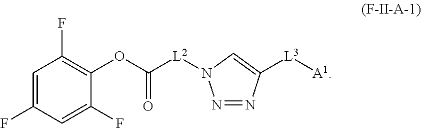

In some embodiments, the compound of formula (F-II-A) is described by formula (F-II-A-2):

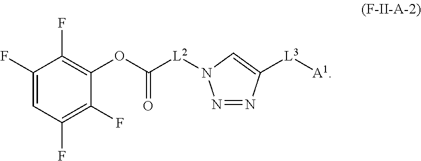

In some embodiments, the compound of formula (G1-A) is described by formula (G1-A-1):

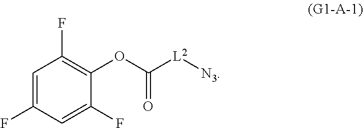

In some embodiments, the compound of formula (G1-A) is described by formula (G1-A-2):

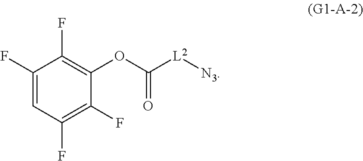

In an aspect, the disclosure features a method of synthesizing a conjugate of formula (M-II):

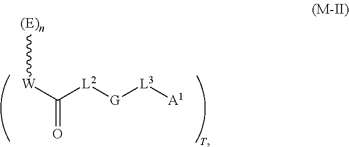

or a pharmaceutically acceptable salt thereof, where n is 1 or 2; W is O, S, NR^{N}, or

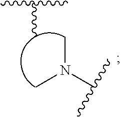

R^{N} is H, optionally substituted C₁-C₂₀ alkylene, or optionally substituted C₁-C₂₀ heteroalkylene;

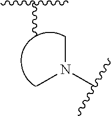

is optionally substituted C₂-C₁₀ heterocyclylene; ach E is a polypeptide or polymer; L² is a linker including one or more of optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ carbocyclylene, optionally substituted C₂-C₂₀ heterocyclylene, optionally substituted C₆-C₂₂ arylene, optionally substituted C₂-C₂₀ heteroarylene, carbonyl, thiocarbonyl, sulfonyl, phosphoryl, optionally substituted amino, O, and S; L³ is a linker including one or more of optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ carbocyclylene, optionally substituted C₂-C₂₀ heterocyclylene, optionally substituted C₆-C₂₂ arylene, optionally substituted C₂-C₂₀ heteroarylene, carbonyl, thiocarbonyl, sulfonyl, phosphoryl, optionally substituted amino, O, and S; G is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted C₃-C₁₀ carbocyclylene, optionally substituted C₂-C₁₀ heterocyclylene, optionally substituted C₆-C₁₀ arylene, or optionally substituted C₂-C₁₀ heteroarylene; each A¹ is a therapeutic agent, or each A¹ is, independently, selected from any one of H, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, and optionally substituted amino; T is an integer from 1 to 20; and each squiggly line in formula (M-II) indicates that

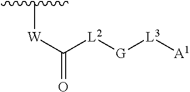

is covalently attached to each E, said method including:
(a) providing a first composition including formula (G3-A) or a salt thereof:

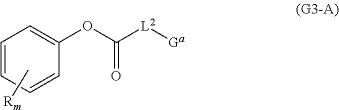

where G^{a} is a functional group that reacts with G^{b} to form G;
(b) providing a second composition including formula (G3-B) or a salt thereof:

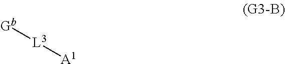

where G^{b} is a functional group that reacts with G^{a} to form G; and
(c) combining the first composition and the second composition to form a first mixture, where m is 0, 1, 2, 3, or 4; and each R is, independently, halo, cyano, nitro, optionally substituted C₁-C₆ alkyl group, or optionally substituted C₁-C₆ heteroalkyl group.

In some embodiments, step (c) includes the use of a Cu(I) source.

In some embodiments, the method further includes:
(d) providing a third composition including E; and
(e) combing the third composition, the first mixture, and a buffer to form a second mixture.

In some embodiments, G^{a} includes optionally substituted amino. In some embodiments, G^{b} includes a carbonyl.

In some embodiments, G^{a} includes a carbonyl. In some embodiments, G^{b} includes optionally substituted amino.

In some embodiments, G^{a} includes an azido group. In some embodiments, G^{b} includes an alkynl group.

In some embodiments, G^{a} includes an alkynyl group. In some embodiments, G^{b} includes an azido group.

In some embodiments, the compound of formula (G3-A) is described by formula (G3-A-1):

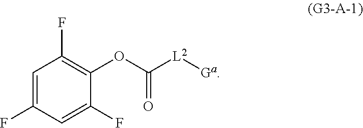

In some embodiments, the compound of formula (G3-A) is described by formula (G3-A-2):

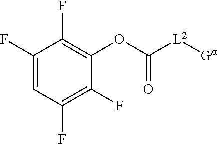

In some embodiments, E is a polypeptide.

In some embodiments, E is an Fc domain monomer, an Fc domain, an Fc-binding peptide, an albumin protein, or an albumin protein-binding peptide.

In some embodiments, E is an Fc domain monomer, an Fc domain, or an Fc-binding peptide. In some embodiments, E is an Fc domain monomer or an Fc domain.

In some embodiments, E includes at least one lysine residue. In some embodiments, the squiggly line in formula (M-I) is covalently bound to a lysine residue of each E. In some embodiments, W is NR^{N}. In some embodiments, R^{N} is H or optionally substituted C₁-C₂₀ alkyl. In some embodiments, R^{N} is H.

In some embodiments, E includes at least one cysteine residue. In some embodiments, the squiggly line in formula (M-I) is covalently bound to a cysteine residue of each E. In some embodiments, W is S.

In some embodiments, E includes at least one proline residue. In some embodiments, the squiggly line in formula (M-I) is covalently bound to a proline residue of each E. In some embodiments, is

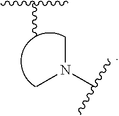

In some embodiments,

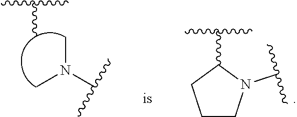

In some embodiments, n is 1. In some embodiments, n is 2.

In some embodiments, E is a polymer.

In some embodiments, E is a polymer derived from one or more species of monomers. In some embodiments, E is a polymer derived from one species of monomer.

In some embodiments, each monomer is, independently, optionally substituted C₁-C₂₀ alkylene (e.g., subunit derived from or including acrylamide), optionally substituted C₁-C₂₀ heteroalkylene (e.g., subunit derived from or including ethylene oxide), optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ carbocyclylene, optionally substituted C₂-C₂₀ heterocyclylene (e.g., saccharide, i.e., carbohydrate (e.g., subunit derived from or including glucose)), optionally substituted C₆-C₂₂ arylene, and optionally substituted C₂-C₂₀ heteroarylene.

In some embodiments, E includes an amine (e.g., NR^{N}R^{N}, where R^{N} is H, optionally substituted C₁-C₂₀ alkyl, or optionally substituted C₁-C₂₀ heteroalkyl), thiol, or hydroxyl. In some embodiments, E includes an amine (e.g., NR^{N}R^{N}, where R^{N} is H, optionally substituted C₁-C₂₀ alkyl, or optionally substituted C₁-C₂₀ heteroalkyl). In some embodiments, E includes —NH₂.

In some embodiments, W is NH.

In some embodiments, A¹ is a therapeutic agent.

In some embodiments, A¹ includes a small molecule. In some embodiments, A¹ includes a monomer, e.g., of a small molecule. In some embodiments, A¹ includes a dimer, e.g., of small molecules. In some embodiments, A¹ includes a monomer or dimer by way of a linker. In some embodiments, A¹ includes a monomer by way of a linker. In some embodiments, A¹ includes a dimer by way of a linker.

In some embodiments, A¹ is a small molecule. In some embodiments, A¹ is a monomer, e.g., of a small molecule. In some embodiments, A¹ is a dimer, e.g., of small molecules. In some embodiments, A¹ is a monomer or dimer by way of a linker. In some embodiments, A¹ is a monomer by way of a linker. In some embodiments, A¹ is a dimer by way of a linker.

In some embodiments, L² is:

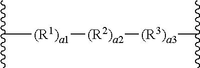

where each of a1, a2, and a3 is, independently, 0 or 1; R¹ is optionally substituted C₁-C₂₀ alkylene, optionally substituted C₁-C₂₀ heteroalkylene, optionally substituted amino, O, or S; R² is optionally substituted C₁-C₂₀ heteroalkylene, optionally substituted C₂-C₂₀ alkenylene, optionally substituted C₂-C₂₀ heteroalkenylene, optionally substituted C₃-C₂₀ cycloalkylene, optionally substituted C₃-C₂₀ heterocycloalkylene, optionally substituted C₆-C₁₈ arylene, or optionally substituted C₂-C₂₀ heteroarylene; and R³ is optionally substituted C₁-C₂₀ alkylene, optionally substituted C₁-C₂₀ heteroalkylene, or carbonyl.

In some embodiments, a1 is 0. In some embodiments, a1 is 1. In some embodiments, a2 is 0. In some embodiments, a2 is 1. In some embodiments, a3 is 0. In some embodiments, a3 is 1.

In some embodiments, a1 is 1 and a3 is 0. In some embodiments, a1 is 1 and a3 is 1.

In some embodiments, R¹ is optionally substituted C₁-C₂₀ alkylene or optionally substituted C₁-C₂₀ heteroalkylene.

In some embodiments, R¹ is optionally substituted C₁-C₂₀ alkylene or optionally substituted C₁-C₂₀ heteroalkylene. In some embodiments, R¹ is optionally substituted C₁-C₂₀ heteroalkylene. In some embodiments, R¹ is C₁-C₂₀ heteroalkylene.

In some embodiments, R¹ is:

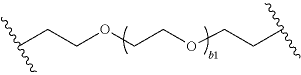

where b1 is 0, 1, 2, 3, 4, 5, 6, 7, or 8.

In some embodiments, R³ is optionally substituted C₁-C₂₀ alkylene or optionally substituted C₁-C₂₀ heteroalkylene. In some embodiments, R³ is optionally substituted C₁-C₂₀ heteroalkylene. In some embodiments, R³ is C₁-C₂₀ heteroalkylene.

In some embodiments, R³ is:

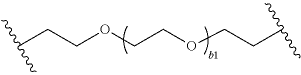

where b1 is 0, 1, 2, 3, 4, 5, 6, 7, or 8.

In some embodiments, L³ is:

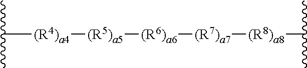

where each of a4, a5, a6, a7, and a8 is, independently, 0 or 1; R⁴ is optionally substituted C₁-C₂₀ alkylene, optionally substituted C₁-C₂₀ heteroalkylene, or carbonyl; R⁵ is optionally substituted C₁-C₂₀ heteroalkylene, optionally substituted C₂-C₂₀ alkenylene, optionally substituted C₂-C₂₀ heteroalkenylene, optionally substituted C₃-C₂₀ cycloalkylene, optionally substituted C₃-C₂₀ heterocycloalkylene, optionally substituted C₆-C₁₈ arylene, optionally substituted C₂-C₂₀ heteroarylene, optionally substituted amino, O, or S; R⁶ is optionally substituted C₁-C₂₀ alkylene, optionally substituted C₁-C₂₀ heteroalkylene, or carbonyl; R⁷ is optionally substituted C₁-C₂₀ heteroalkylene, optionally substituted C₂-C₂₀ alkenylene, optionally substituted C₂-C₂₀ heteroalkenylene, optionally substituted C₃-C₂₀ cycloalkylene, optionally substituted C₃-C₂₀ heterocycloalkylene, optionally substituted C₆-C₁₈ arylene, optionally substituted C₂-C₂₀ heteroarylene, optionally substituted amino, O, or S; and R⁸ is optionally substituted C₁-C₂₀ alkylene, optionally substituted C₁-C₂₀ heteroalkylene, or carbonyl.

In some embodiments, a4 is 0. In some embodiments, a4 is 1. In some embodiments, a5 is 0. In some embodiments, a5 is 1. In some embodiments, a6 is 0. In some embodiments, a6 is 1. In some embodiments, a7 is 0. In some embodiments, a7 is 1. In some embodiments, a8 is 0. In some embodiments, a8 is 1.

In some embodiments, a4 is 1, a5 is 1, a6 is 1, a7 is 1, and a8 is 1.

In some embodiments, R⁴ is optionally substituted C₁-C₂₀ alkylene or optionally substituted C₁-C₂₀ heteroalkylene.

In some embodiments, R⁴ is:

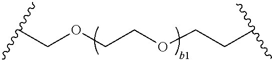

where b1 is 0, 1, 2, 3, 4, 5, 6, 7, or 8.

In some embodiments, R⁵ is optionally substituted amino or optionally substituted C₃-C₂₀ heterocycloalkylene.

In some embodiments, R⁶ is optionally substituted C₁-C₂₀ alkylene.

In some embodiments, R⁷ is optionally substituted amino.

In some embodiments, R⁸ is carbonyl.

In some embodiments, each R is, independently, halo, cyano, nitro, haloalkyl, or

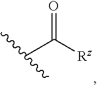

where R^{z} is optionally substituted C₁-C₅ alkyl group or optionally substituted C₁-C₅ heteroalkyl group. In some embodiments, each R is, independently, halo, cyano, nitro, or haloalkyl.

In some embodiments, each R is, independently, F, Cl, Br, or I.

In some embodiments, each R is F.

In some embodiments, m is 1, 2, 3, 4, or 5. In some embodiments, m is 3, 4, or 5. In some embodiments, m is 3 or 4. In some embodiments, m is 3. In some embodiments, m is 4.

In some embodiments,

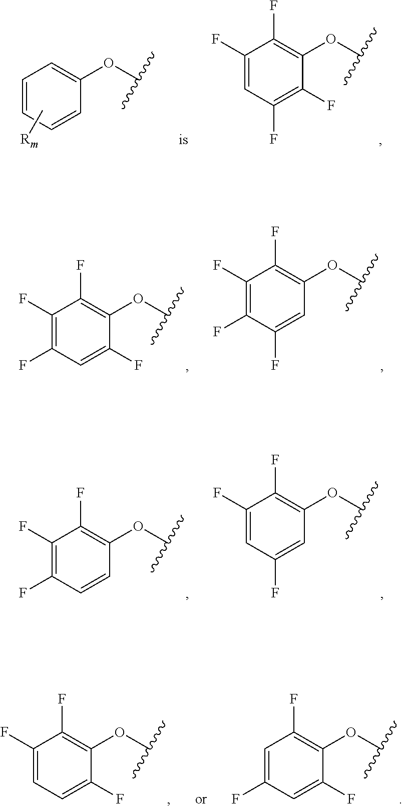

In some embodiments,

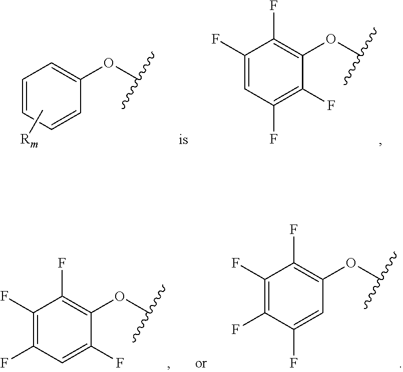

In some embodiments,

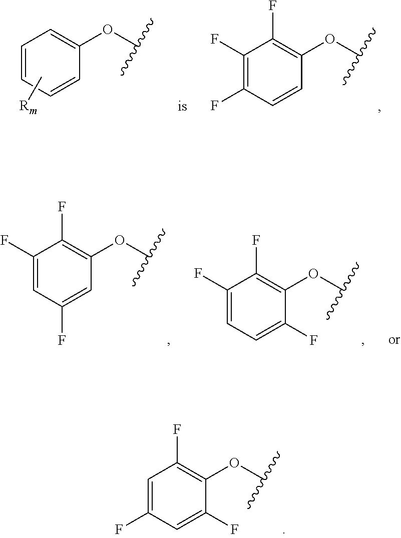

In some embodiments,

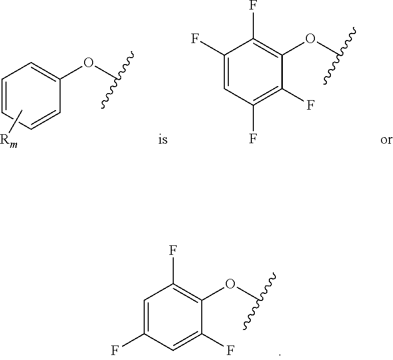

In some embodiments,

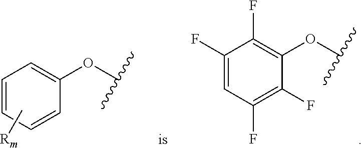

In some embodiments,

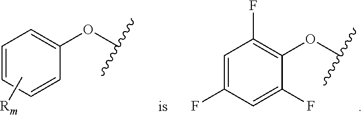

In some embodiments, a compound of formula (F-II) (e.g., a compound of formula (F-II-A) or (F-II-B) and/or a compound of formula (G1-A) or (G2-A), where each R is halo (e.g., F), provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In some embodiments, a compound of formula (F-II) (e.g., a compound of formula (F-II-A) or (F-II-B) and/or a compound of formula (G1-A) or (G2-A), where m is 3, provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In some embodiments, a compound of formula (F-II) (e.g., a compound of formula (F-II-A) or (F-II-B) and/or a compound of formula (G1-A) or (G2-A), where m is 3 and each R is halo (e.g., F), provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In some embodiments, the buffer includes borate or carbonate. In some embodiments, the buffer includes borate. In some embodiments, the buffer includes carbonate.

In some embodiments, the buffer has a pH of about 7.0 to 10.0 (e.g., about 7.0 to 7.5, 7.5 to 8.0, 8.0 to 8.5, 8.5 to 9.0, 9.0 to 9.5, 9.5 to 10.0, 7.0 to 8.0, 7.5 to 8.5, 8.0 to 9.0, 8.5 to 9.5, 9.0 to 10.0, 7.0 to 9.0, 7.5 to 9.5, or 8.0 to 10.0).

In some embodiments, the buffer has a pH of about 7.0. In some embodiments, the buffer has a pH of about 7.1. In some embodiments, the buffer has a pH of about 7.2. In some embodiments, the buffer has a pH of about 7.3. In some embodiments, the buffer has a pH of about 7.4. In some embodiments, the buffer has a pH of about 7.5. In some embodiments, the buffer has a pH of about 7.6. In some embodiments, the buffer has a pH of about 7.7. In some embodiments, the buffer has a pH of about 7.8. In some embodiments, the buffer has a pH of about 7.9. In some embodiments, the buffer has a pH of about 8.0. In some embodiments, the buffer has a pH of about 8.1. In some embodiments, the buffer has a pH of about 8.2. In some embodiments, the buffer has a pH of about 8.3. In some embodiments, the buffer has a pH of about 8.4. In some embodiments, the buffer has a pH of about 8.5. In some embodiments, the buffer has a pH of about 8.6. In some embodiments, the buffer has a pH of about 8.7. In some embodiments, the buffer has a pH of about 8.8. In some embodiments, the buffer has a pH of about 8.9. In some embodiments, the buffer has a pH of about 9.0. In some embodiments, the buffer has a pH of about 9.5. In some embodiments, the buffer has a pH of about 9.6. In some embodiments, the buffer has a pH of about 9.7. In some embodiments, the buffer has a pH of about 9.8. In some embodiments, the buffer has a pH of about 9.9. In some embodiments, the buffer has a pH of about 10.0.

In some embodiments, step (c) is conducted at a temperature of 5 to 50° C., such as 20 to 30° C. (e.g., 20 to 25, 21 to 26, 22 to 27, 23 to 28, 24 to 29, or 25 to 30° C.).

In some embodiments, step (c) is conducted at a temperature of about 25° C.

In some embodiments, step (c) is conducted for about 1 to 24 hours, such as 1 to 12 hours (e.g., 1 to 2, 1 to 5, 2 to 3, 2 to 5, 2 to 10, 2 to 12, 3 to 4, 4 to 5, 1 to 3, 2 to 4, or 3 to 5 hours).

In some embodiments, step (c) is conducted for about 2 hours. In some embodiments, step (c) is conducted for about 3 hours. In some embodiments, step (c) is conducted for about 4 hours. In some embodiments, step (c) is conducted for about 5 hours. In some embodiments, step (c) is conducted for about 6 hours. In some embodiments, step (c) is conducted for about 7 hours. In some embodiments, step (c) is conducted for about 8 hours. In some embodiments, step (c) is conducted for about 9 hours. In some embodiments, step (c) is conducted for about 10 hours. In some embodiments, step (c) is conducted for about 11 hours. In some embodiments, step (c) is conducted for about 12 hours.

In some embodiments, the first composition includes phosphate-buffered saline buffer.

In some embodiments, the buffer has a pH of about 7.0 to 8.0 (e.g., about 7.0 to 7.5, 7.5 to 8.0, 7.0 to 7.2, 7.2 to 7.4, 7.4 to 7.6, 7.6 to 7.8, or 7.8 to 8.0).

In some embodiments, the buffer has a pH of about 7.5.

In some embodiments, the second composition includes DMF.

In some embodiments, the method further includes a purification step. In some embodiments, the purification step includes dialysis in arginine buffer. In some embodiments, the purification step includes a buffer exchange.

In some embodiments, T is an integer from 1 to 20 (e.g., 1, 2, 3, 4, 5, 6, 7, 8, 9, 10, 11, 12, 13, 14, 15, 16, 17, 18, 19, or 20). In some embodiments, the average value of T is 1 to 20 (e.g., the average value of T is 1 to 2, 1 to 3, 1 to 4, 1 to 5, 5 to 10, 10 to 15, or 15 to 20). In some embodiments, the average value of T is 1, 2, 3, 4, 5, 6, 7, 8, 9, 10, 11, 12, 13, 14, 15, 16, 17, 18, 19, or 20. In certain embodiments, the average T is 1 to 10 (e.g., 1.5, 2, 2.5, 3, 3.5, 4, 4.5, 5, 5.5, 6, 6.5, 7, 7.5, 8, 8.5, 9, 9.5, or 10). In certain embodiments, the average T is 1 to 5 (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5). In some embodiment, the average T is 5 to 10 (e.g., 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10). In some embodiments, the average T is 2.5 to 7.5 (e.g., 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5).

In an aspect, the disclosure features a conjugate produced by any of the methods described herein. In some embodiments, T is an integer from 1 to 20 (e.g., 1, 2, 3, 4, 5, 6, 7, 8, 9, 10, 11, 12, 13, 14, 15, 16, 17, 18, 19, or 20). In some embodiments, the conjugate produced by any of the methods described herein has average T value of 1 to 20 (e.g., the average value of T is 1 to 2, 1 to 3, 1 to 4, 1 to 5, 5 to 10, 10 to 15, or 15 to 20). In some embodiments, the average value of T is 1 to 20 (e.g., the average value of T is 1 to 2, 1 to 3, 1 to 4, 1 to 5, 5 to 10, 10 to 15, or 15 to 20). In some embodiments, the average value of T is 1, 2, 3, 4, 5, 6, 7, 8, 9, 10, 11, 12, 13, 14, 15, 16, 17, 18, 19, or 20. In certain embodiments, the average T is 1 to 10 (e.g., 1.5, 2, 2.5, 3, 3.5, 4, 4.5, 5, 5.5, 6, 6.5, 7, 7.5, 8, 8.5, 9, 9.5, or 10). In certain embodiments, the average T is 1 to 5 (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5). In some embodiment, the average T is 5 to 10 (e.g., 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10). In some embodiments, the average T is 2.5 to 7.5 (e.g., 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5).

In an aspect, the disclosure features a population of conjugates produced by any of the methods described herein. In some embodiments, T is an integer from 1 to 20 (e.g., 1, 2, 3, 4, 5, 6, 7, 8, 9, 10, 11, 12, 13, 14, 15, 16, 17, 18, 19, or 20). In some embodiments, a population of any of the conjugates produced by any of the methods described herein has average T value of 1 to 20 (e.g., the average value of T is 1 to 2, 1 to 3, 1 to 4, 1 to 5, 5 to 10, 10 to 15, or 15 to 20). In some embodiments, the average value of T is 1, 2, 3, 4, 5, 6, 7, 8, 9, 10, 11, 12, 13, 14, 15, 16, 17, 18, 19, or 20. In certain embodiments, the average T is 1 to 10 (e.g., 1.5, 2, 2.5, 3, 3.5, 4, 4.5, 5, 5.5, 6, 6.5, 7, 7.5, 8, 8.5, 9, 9.5, or 10). In certain embodiments, the average T is 1 to 5 (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5). In some embodiment, the average T is 5 to 10 (e.g., 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10). In some embodiments, the average T is 2.5 to 7.5 (e.g., 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5).

### Definitions

As used herein, the term “about” refers to a range of values that is ±10% of specific value. For example, “about 150 mg” includes ±10% of 150 mg, or from 135 mg to 165 mg. Such a range performs the desired function or achieves the desired result. For example, “about” may refer to an amount that is within less than 10% of, within less than 5% of, within less than 1% of, within less than 0.1% of, and within less than 0.01% of the stated amount.

As used herein, the term “between” refers to any quantity within the range indicated and enclosing each of the ends of the range indicated. For example, a pH of between 5 and 7 refers to any quantity within 5 and 7, as well as a pH of 5 and a pH of 7.

Any values provided in a range of values include both the upper and lower bounds, and any values contained within the upper and lower bounds.

The term “covalently attached” refers to two parts of a conjugate that are linked to each other by a covalent bond formed between two atoms in the two parts of the conjugate.

As used herein, the term “percent (%) identity” refers to the percentage of amino acid residues of a candidate sequence, e.g., an Fc-IgG, or fragment thereof, that are identical to the amino acid residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid sequence identity of a given candidate sequence to, with, or against a given reference sequence (which can alternatively be phrased as a given candidate sequence that has or includes a certain percent amino acid sequence identity to, with, or against a given reference sequence) is calculated as follows:

100×(fraction of A/B)

where A is the number of amino acid residues scored as identical in the alignment of the candidate sequence and the reference sequence, and where B is the total number of amino acid residues in the reference sequence. In some embodiments where the length of the candidate sequence does not equal to the length of the reference sequence, the percent amino acid sequence identity of the candidate sequence to the reference sequence would not equal to the percent amino acid sequence identity of the reference sequence to the candidate sequence.

Two polynucleotide or polypeptide sequences are said to be “identical” if the sequence of nucleotides or amino acids in the two sequences is the same when aligned for maximum correspondence as described above. Comparisons between two sequences are typically performed by comparing the sequences over a comparison window to identify and compare local regions of sequence similarity. A “comparison window” as used herein, refers to a segment of at least about 15 contiguous positions, about 20 contiguous positions, about 25 contiguous positions, or more (e.g., about 30 to about 75 contiguous positions, or about 40 to about 50 contiguous positions), in which a sequence may be compared to a reference sequence of the same number of contiguous positions after the two sequences are optimally aligned.

As used herein, the term “X ester” refers to an ester including the group X (e.g., “tetrafluorophenyl ester” refers to an ester including a tetrafluorophenyl group).

As used herein, the term “small molecule” refers to a low molecular weight compound (e.g., a compound (e.g., an organic compound) having less than 900 Da, that may regulate a biological process, with a size on the order of 1 nm. In some instances, a therapeutic agent is a small molecule therapeutic agent. In some instances, the small molecule agent is between about 300 and about 700 Da (e.g., about 325 Da, about 350 Da, about 375 Da, about 400 Da, about 425 Da, about 450 Da, about 475 Da, about 500 Da, about 525 Da, about 550 Da, about 575 Da, about 600 Da, about 625 Da, about 650 Da, or about 675 Da).

As used-herein, a “surface exposed amino acid” or “solvent-exposed amino acid,” such as a surface exposed cysteine or a surface exposed lysine refers to an amino acid that is accessible to the solvent surrounding the protein. A surface exposed amino acid may be a naturally-occurring or an engineered variant (e.g., a substitution or insertion) of the protein. In some embodiments, a surface exposed amino acid is an amino acid that when substituted does not substantially change the three-dimensional structure of the protein.

The term “subject,” as used herein, can be a human or non-human primate, or other mammal, such as but not limited to dog, cat, horse, cow, pig, turkey, goat, fish, monkey, chicken, rat, mouse, or sheep.

As used herein, the term “Fc domain monomer” refers to a polypeptide chain that includes at least a hinge domain and second and third antibody constant domains (C_{H}2 and C_{H}3) or functional fragments thereof (e.g., fragments that that capable of (i) dimerizing with another Fc domain monomer to form an Fc domain, and (ii) binding to an Fc receptor. The Fc domain monomer can be any immunoglobulin antibody isotype, including IgG, IgE, IgM, IgA, or IgD (e.g., IgG). Additionally, the Fc domain monomer can be an IgG subtype (e.g., IgG1, IgG2a, IgG2b, IgG3, or IgG4) (e.g., IgG1). An Fc domain monomer does not include any portion of an immunoglobulin that is capable of acting as an antigen-recognition region, e.g., a variable domain or a complementarity determining region (CDR). Fc domain monomers in the conjugates as described herein can contain one or more changes from a wild-type Fc domain monomer sequence (e.g., 1-10, 1-8, 1-6, 1-4 amino acid substitutions, additions, or deletions) that alter the interaction between an Fc domain and an Fc receptor. Examples of suitable changes are known in the art. In certain embodiments, a human Fc domain monomer (e.g., an IgG heavy chain, such as IgG1) includes a region that extends from any of Asn201 or Glu216 (e.g., Asn201, Val 202, Asn203, His204, Lys 205, Pro206, Ser207, Asn208, Thr209, Lys210, Val211, Asp212, Lys 213, Lys214, Val215, or Glu216), to the carboxyl-terminus of the heavy chain, e.g., at Gly446 or Lys447. C-terminal Lys447 of the Fc region may or may not be present, without affecting the structure or stability of the Fc region. C-terminal Lys447 of the Fc region may or may not be present, without affecting the structure or stability of the Fc region. C-terminal Lys 447 may be proteolytically cleaved upon expression of the polypeptide. In some embodiments of any of the Fc domain monomers described herein, C-terminal Lys 447 is optionally present or absent. The N-terminal N (Asn) of the Fc region may or may not be present, without affecting the structure of stability of the Fc region. N-terminal Asn may be deamidated upon expression of the polypeptide. In some embodiments of any of the Fc domain monomers described herein, N-terminal Asn is optionally present or absent. Unless otherwise specified herein, numbering of amino acid residues in the IgG or Fc domain monomer is according to the EU numbering system for antibodies, also called the Kabat EU index, as described, for example, in Kabat et al., Sequences of Proteins of Immunological Interest, 5th Ed. Public Health Service, National Institutes of Health, Bethesda, M D, 1991.

As used herein, the term “Fc domain” refers to a dimer of two Fc domain monomers that is capable of binding an Fc receptor. In the wild-type Fc domain, the two Fc domain monomers dimerize by the interaction between the two C_{H}3 antibody constant domains, in some embodiments, one or more disulfide bonds form between the hinge domains of the two dimerizing Fc domain monomers.

As used herein, the term “Fc-binding peptide” refers to refers to a polypeptide having an amino acid sequence of 5 to 50 (e.g., 5 to 40, 5 to 30, 5 to 20, 5 to 15, 5 to 10, 10 to 50, 10 to 30, or 10 to 20) amino acid residues that has affinity for and functions to bind an Fc domain, such as any of the Fc domain described herein. An Fc-binding peptide can be of different origins, e.g., synthetic, human, mouse, or rat. Fc-binding peptides of the disclosure include Fc-binding peptides which have been engineered to include one or more (e.g., two, three, four, or five) solvent-exposed cysteine or lysine residues, which may provide a site for conjugation to a compound of the disclosure (e.g., a compound of formula (F-I) or (F-II)). Most preferably, the Fc-binding peptide will contain a single solvent-exposed cysteine or lysine, thus enabling site-specific conjugation of a compound of the disclosure. Fc-binding peptides may include only naturally occurring amino acid residues, or may include one or more non-naturally occurring amino acid residues. Where included, a non-naturally occurring amino acid residue (e.g., the side chain of a non-naturally occurring amino acid residue) may used as the point of attachment for a compound of formula (F-I) or (F-II). Fc-binding peptides of the disclosure may be linear or cyclic. Fc-binding peptides of the disclosure include any Fc-binding peptides known to one of skill in the art.

As used here, the term “albumin protein” refers to a polypeptide including an amino acid sequence corresponding to a naturally-occurring albumin protein (e.g., human serum albumin) or a variant thereof, such as an engineered variant of a naturally-occurring albumin protein. Variants of albumin proteins include polymorphisms, fragments such as domains and sub-domains, and fusion proteins (e.g., an albumin protein having a C-terminal or N-terminal fusion, such as a polypeptide linker). Preferably the albumin protein has the amino acid sequence of human serum albumin (HSA) or a variant or fragment thereof, most preferably a functional variant or fragment thereof Albumin proteins of the disclosure include albumin proteins which have been engineered to include one or more (e.g., two, three, four, or five) solvent-exposed cysteine or lysine residues, which may provide a site for conjugation to a compound of formula (F-I) or (F-II). Most preferably, the albumin protein will contain a single solvent-exposed cysteine or lysine, thus enabling site-specific conjugation of a compound of the disclosure. Albumin proteins may include only naturally occurring amino acid residues, or may include one or more non-naturally occurring amino acid residues. Where included, a non-naturally occurring amino acid residue (e.g., the side chain of a non-naturally occurring amino acid residue) may used as the point of attachment for a compound of formula (F-I) or (F-II).

As used herein, the term “albumin protein-binding peptide” refers to a polypeptide having an amino acid sequence of 5 to 50 (e.g., 5 to 40, 5 to 30, 5 to 20, 5 to 15, 5 to 10, 10 to 50, 10 to 30, or 10 to 20) amino acid residues that has affinity for and functions to bind an albumin protein, such as any of the albumin proteins described herein. Preferably, the albumin protein-binding peptide binds to a naturally-occurring serum albumin, most preferably human serum albumin. An albumin protein-binding peptide can be of different origins, e.g., synthetic, human, mouse, or rat. Albumin protein-binding peptides of the disclosure include albumin protein-binding peptides which have been engineered to include one or more (e.g., two, three, four, or five) solvent-exposed cysteine or lysine residues, which may provide a site for conjugation to a compound of formula (F-I) or (F-II). Most preferably, the albumin protein-binding peptide will contain a single solvent-exposed cysteine or lysine, thus enabling site-specific conjugation of a compound of the disclosure. Albumin protein-binding peptides may include only naturally occurring amino acid residues, or may include one or more non-naturally occurring amino acid residues. Where included, a non-naturally occurring amino acid residue (e.g., the side chain of a non-naturally occurring amino acid residue) may be used as the point of attachment for a compound of formula (F-I) or (F-II). Albumin protein-binding peptides of the disclosure may be linear or cyclic. Albumin protein-binding peptide of the disclosure include any albumin protein-binding peptides known to one of skill in the art, examples of which, are provided herein. Further exemplary albumin protein-binding peptides are provided in U.S. Patent Application No. 2005/0287153, which is incorporated herein by reference in its entirety.

The term “linker,” as used herein, refers to a covalent linkage or connection between two or more components in a conjugate described herein (e.g., between W and A¹, between W and G, between G and A¹, and/or between a compound of formula (F-I) or (F-II) and E).

Molecules that may be used as linkers include at least two functional groups, which may be the same or different, e.g., two carboxylic acid groups, two amine groups, two sulfonic acid groups, a carboxylic acid group and a maleimide group, a carboxylic acid group and an alkyne group, a carboxylic acid group and an amine group, a carboxylic acid group and a sulfonic acid group, an amine group and a maleimide group, an amine group and an alkyne group, or an amine group and a sulfonic acid group. The first functional group may form a covalent linkage with a first component in the conjugate and the second functional group may form a covalent linkage with the second component in the conjugate. In some embodiments, a molecule containing one or more maleimide groups may be used as a linker, in which the maleimide group may form a carbon-sulfur linkage with a cysteine in a component in the conjugate. In some embodiments, a molecule containing one or more alkyne groups may be used as a linker, in which the alkyne group may form a 1,2,3-triazole linkage with an azide in a component in the conjugate. In some embodiments, a molecule containing one or more azide groups may be used as a linker, in which the azide group may form a 1,2,3-triazole linkage with an alkyne in a component in the conjugate. In some embodiments, a molecule containing one or more bis-sulfone groups may be used as a linker, in which the bis-sulfone group may form a linkage with an amine group a component in the conjugate. In some embodiments, a molecule containing one or more sulfonic acid groups may be used as a linker, in which the sulfonic acid group may form a sulfonamide linkage with a component in the conjugate. In some embodiments, a molecule containing one or more isocyanate groups may be used as a linker, in which the isocyanate group may form a urea linkage with a component in the conjugate. In some embodiments, a molecule containing one or more haloalkyl groups may be used as a linker, in which the haloalkyl group may form a covalent linkage, e.g., C—N and C—O linkages, with a component in the conjugate.

In some embodiments, a molecule containing one or more phenyl ester groups (e.g., trifluorophenyl ester groups or tetrafluorophenyl ester groups) may be used as a linker, in which the phenyl ester group (e.g., trifluorophenyl ester group or tetrafluorophenyl ester group) may form an amide with an amine in a component (e.g., a polypeptide) in the conjugate.

In some embodiments, a linker provides space, rigidity, and/or flexibility between the two or more components. In some embodiments, a linker may be a bond, e.g., a covalent bond. The term “bond” refers to a chemical bond, e.g., an amide bond, a disulfide bond, a C—O bond, a C—N bond, a N—N bond, a C—S bond, or any kind of bond created from a chemical reaction, e.g., chemical conjugation. In some embodiments, a linker includes no more than 250 atoms. In some embodiments, a linker includes no more than 250 non-hydrogen atoms. In some embodiments, the backbone of a linker includes no more than 250 atoms. The “backbone” of a linker refers to the atoms in the linker that together form the shortest path from one part of a conjugate to another part of the conjugate (e.g., the shortest path linking a polypeptide and a therapeutic agent). The atoms in the backbone of the linker are directly involved in linking one part of a conjugate to another part of the conjugate (e.g., linking a polypeptide and a therapeutic agent). For examples, hydrogen atoms attached to carbons in the backbone of the linker are not considered as directly involved in linking one part of the conjugate to another part of the conjugate.

In some embodiments, a linker may comprise a synthetic group derived from, e.g., a synthetic polymer (e.g., a polyethylene glycol (PEG) polymer). In some embodiments, a linker may comprise one or more amino acid residues, such as D- or L-amino acid residues. In some embodiments, a linker may be a residue of an amino acid sequence (e.g., a 1-25 amino acid, 1-10 amino acid, 1-9 amino acid, 1-8 amino acid, 1-7 amino acid, 1-6 amino acid, 1-5 amino acid, 1-4 amino acid, 1-3 amino acid, 1-2 amino acid, or 1 amino acid sequence). In some embodiments, a linker may comprise one or more, e.g., 1-100, 1-50, 1-25, 1-10, 1-5, or 1-3, optionally substituted alkylene, optionally substituted heteroalkylene (e.g., a PEG unit), optionally substituted alkenylene, optionally substituted heteroalkenylene, optionally substituted alkynylene, optionally substituted heteroalkynylene, optionally substituted cycloalkylene, optionally substituted heterocycloalkylene, optionally substituted cycloalkenylene, optionally substituted heterocycloalkenylene, optionally substituted cycloalkynylene, optionally substituted heterocycloalkynylene, optionally substituted arylene, optionally substituted heteroarylene (e.g., pyridine), O, S, NRⁱ,

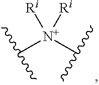

(each Rⁱ is, independently, H, optionally substituted alkyl, optionally substituted heteroalkyl, optionally substituted alkenyl, optionally substituted heteroalkenyl, optionally substituted alkynyl, optionally substituted heteroalkynyl, optionally substituted cycloalkyl, optionally substituted heterocycloalkyl, optionally substituted cycloalkenyl, optionally substituted heterocycloalkenyl, optionally substituted cycloalkynyl, optionally substituted heterocycloalkynyl, optionally substituted aryl, or optionally substituted heteroaryl), P, carbonyl, thiocarbonyl, sulfonyl, phosphate, phosphoryl, or imino. For example, a linker may comprise one or more optionally substituted C₁-C₂₀ alkylene, optionally substituted C₁-C₂₀ heteroalkylene (e.g., a PEG unit), optionally substituted C₂-C₂₀ alkenylene (e.g., C₂ alkenylene), optionally substituted C₂-C₂₀ heteroalkenylene, optionally substituted C₂-C₂₀ alkynylene, optionally substituted C₂-C₂₀ heteroalkynylene, optionally substituted C₃-C₂₀ cycloalkylene (e.g., cyclopropylene, cyclobutylene), optionally substituted C₂-C₂₀ heterocycloalkylene, optionally substituted C₄-C₂₀ cycloalkenylene, optionally substituted C₄-C₂₀ heterocycloalkenylene, optionally substituted C₈-C₂₀ cycloalkynylene, optionally substituted C₈-C₂₀ heterocycloalkynylene, optionally substituted C₅-C₁₅ arylene (e.g., C₆ arylene), optionally substituted C₃-C₁₅ heteroarylene (e.g., imidazole, pyridine), O, S, NRⁱ,

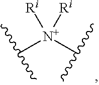

(each Rⁱ is, independently, H, optionally substituted C₁-C₂₀ alkyl, optionally substituted C₁-C₂₀ heteroalkyl, optionally substituted C₂-C₂₀ alkenyl, optionally substituted C₂-C₂₀ heteroalkenyl, optionally substituted C₂-C₂₀ alkynyl, optionally substituted C₂-C₂₀ heteroalkynyl, optionally substituted C₃-C₂₀ cycloalkyl, optionally substituted C₂-C₂₀ heterocycloalkyl, optionally substituted C₄-C₂₀ cycloalkenyl, optionally substituted C₄-C₂₀ heterocycloalkenyl, optionally substituted C₈-C₂₀ cycloalkynyl, optionally substituted C₈-C₂₀ heterocycloalkynyl, optionally substituted C₅-C₁₅ aryl, or optionally substituted C₃-C₁₅ heteroaryl), P, carbonyl, thiocarbonyl, sulfonyl, phosphate, phosphoryl, or imino.

As used herein, the term “polymer” refers to a molecule comprising repeating structural subunits (e.g., monomers). Examples of monomers include optionally substituted C₁-C₂₀ alkylene (e.g., subunit derived from or including acrylamide), optionally substituted C₁-C₂₀ heteroalkylene (e.g., subunit derived from or including ethylene oxide), and optionally substituted C₂-C₂₀ heterocyclylene (e.g., saccharide, i.e., carbohydrate (e.g., subunit derived from or including glucose)). Polymers may be synthetic or natural. A polymer can be derived from one species of monomer (i.e., a homopolymer) or more than one species of monomer (i.e., a copolymer). Polymers can include ten or more (e.g., fifteen or more, twenty or more, twenty-five or more, thirty or more, thirty-five or more, forty or more, forty-five or more, fifty or more, or a hundred or more) monomers. Exemplary polymers include polyacrylamides, polyethylene glycols, and polysaccharides, i.e., polycarbohydrates (e.g., dextran). Polymers can be soluble in water or aqueous buffer. Polymers can also be safely administered in a subject (e.g., animal (e.g., humans)). Additionally, polymers can also include reactive groups, e.g., optionally substituted amine (e.g., NR^{N}R^{N}, where each R^{N} is, independently, H, optionally substituted C₁-C₂₀ alkyl, or optionally substituted C₁-C₂₀ heteroalkyl), thiol, or hydroxyl.

The term “polypeptide,” as used herein, refers to a polymer of amino acid residues. Polypeptides of the present disclosure can be composed of any continuous peptide chain including ten or more (e.g., fifteen or more, twenty or more, twenty-five or more, thirty or more, thirty-five or more, forty or more, forty-five or more, fifty or more, or a hundred or more) amino acids (e.g., naturally occurring amino acids and/or non-naturally occurring amino acids).

### Chemical Terms

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C alkyl. Furthermore, where a compound includes a plurality of positions at which substitutes are disclosed in groups or in ranges, unless otherwise indicated, the present disclosure is intended to cover individual compounds and groups of compounds (e.g., genera and subgenera) containing each and every individual subcombination of members at each position.

The term “optionally substituted,” as used herein, refers to having 0, 1, or more substituents, such as 0-25, 0-20, 0-10 or 0-5 substituents. Substituents include, but are not limited to, alkyl, alkenyl, alkynyl, aryl, carbocyclyl (e.g., cycloalkyl, cycloalkenyl, or cycloalkynyl), alkaryl, acyl, heteroaryl, heterocyclyl (e.g., heteroalkyl, heteroalkenyl, or heteroalkynyl), heteroalkaryl, halogen, oxo, cyano, nitro, amino, alkamino, hydroxy, alkoxy, alkanoyl, carbonyl, carbamoyl, guanidinyl, ureido, amidinyl, any of the groups or moieties described herein, and hetero versions of any of the groups or moieties described herein. Substituents include, but are not limited to, F, Cl, Br, I, halogenated alkyl, methyl, phenyl, benzyl, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, OCF₃, SiR₃, and NO₂, wherein each R is, independently, H, alkyl, alkenyl, aryl, heteroalkyl, heteroalkenyl, or heteroaryl, and wherein two of the optional substituents on the same or adjacent atoms can be joined to form a fused, optionally substituted aromatic or nonaromatic, saturated or unsaturated ring which contains 3-8 members, or two of the optional substituents on the same atom can be joined to form an optionally substituted aromatic or nonaromatic, saturated or unsaturated ring which contains 3-8 members.

An optionally substituted group or moiety refers to a group or moiety (e.g., any one of the groups or moieties described above) in which one of the atoms (e.g., a hydrogen atom) is optionally replaced with another substituent. For example, an optionally substituted alkyl may be an optionally substituted methyl, in which a hydrogen atom of the methyl group is replaced by, e.g., OH. As another example, a substituent on a heteroalkyl or its divalent counterpart, heteroalkylene, may replace a hydrogen on a carbon or a hydrogen on a heteroatom such as N. For example, the hydrogen atom in the group —R—NH—R— may be substituted with an alkamide substituent, e.g., —R—N[(CH₂C(O)N(CH₃)₂]—R.

The term “acyl,” as used herein, refers to a group having the structure:

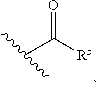

wherein R^{z} is an optionally substituted alkyl, alkenyl, alkynyl, carbocyclyl (e.g., cycloalkyl, cycloalkenyl, or cycloalkynyl), aryl, alkaryl, alkamino, heteroalkyl, heteroalkenyl, heteroalkynyl, heterocyclyl (e.g., heterocycloalkyl, heterocycloalkenyl, or heterocycloalkynyl), heteroaryl, heteroalkaryl, or heteroalkamino. An example of optionally substituted alkyl group is an acyl group where R^{z} is optionally substituted alkyl. An example of optionally substituted heteroalkyl group is an acyl group where R^{z} is optionally substituted heteroalkyl.

The terms “alkyl,” “alkenyl,” and “alkynyl,” as used herein, include straight-chain and branched-chain monovalent substituents, as well as combinations of these, containing only C and H when unsubstituted. When the alkyl group includes at least one carbon-carbon double bond or carbon-carbon triple bond, the alkyl group can be referred to as an “alkenyl” or “alkynyl” group, respectively. The monovalency of an alkyl, alkenyl, or alkynyl group does not include the optional substituents on the alkyl, alkenyl, or alkynyl group. For example, if an alkyl, alkenyl, or alkynyl group is attached to a compound, monovalency of the alkyl, alkenyl, or alkynyl group refers to its attachment to the compound and does not include any additional substituents that may be present on the alkyl, alkenyl, or alkynyl group. Alkyl, alkenyl, and alkynyl groups may be optionally substituted. Substituents include, but are not limited to, alkyl, alkenyl, alkynyl, aryl, carbocyclyl (e.g., cycloalkyl, cycloalkenyl, or cycloalkynyl), alkaryl, acyl, heteroaryl, heterocyclyl (e.g., heteroalkyl, heteroalkenyl, or heteroalkynyl), heteroalkaryl, halogen, oxo, cyano, nitro, amino, alkamino, hydroxy, alkoxy, alkanoyl, carbonyl, carbamoyl, guanidinyl, ureido, amidinyl, any of the groups or moieties described herein, and hetero versions of any of the groups or moieties described herein. Substituents also include F, Cl, Br, I, halogenated alkyl, methyl, phenyl, benzyl, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, OCF₃, SiR₃, and NO₂, wherein each R is, independently, H, alkyl, alkenyl, aryl, heteroaryl, carbocyclyl, or heterocyclyl and wherein two of the optional substituents on the same or adjacent atoms can be joined to form a fused, optionally substituted aromatic or nonaromatic, saturated or unsaturated ring which contains 3-8 members, or two of the optional substituents on the same atom can be joined to form an optionally substituted aromatic or nonaromatic, saturated or unsaturated ring which contains 3-8 members.

The term “hetero,” when used to describe a chemical group or moiety, refers to having at least one heteroatom that is not a carbon or a hydrogen, e.g., N, O, and S. Any one of the groups or moieties described herein may be referred to as hetero if it contains at least one heteroatom. For example, a heterocycloalkyl, heterocycloalkenyl, or heterocycloalkynyl group refers to a cycloalkyl, cycloalkenyl, or cycloalkynyl group that has one or more heteroatoms independently selected from, e.g., N, O, and S For example, a heteroaryl ring refers to an aromatic ring that has one or more heteroatoms independently selected from, e.g., N, O, and S. One or more heteroatoms may also be included in a substituent that replaced a hydrogen atom in a group or moiety as described herein. For example, in an optionally substituted heteroaryl group, if one of the hydrogen atoms in the heteroaryl group is replaced with a substituent (e.g., methyl), the substituent may also contain one or more heteroatoms (e.g., methanol). In some embodiments, the alkyl or heteroalkyl group may contain, e.g., 1-20. 1-18, 1-16, 1-14, 1-12, 1-10, 1-8, 1-6, 1-4, or 1-2 carbon atoms (e.g., C₁-C₂₀, C₁-C₁₈, C₁-C₁₆, C₁-C₁₄, C₁-C₁₂, C₁-C₁₀, C₁-C₈, C₁-C₆, C₁-C₄, or C₁-C₂). In some embodiments, the alkenyl, heteroalkenyl, alkynyl, or heteroalkynyl group may contain, e.g., 2-20, 2-18, 2-16, 2-14, 2-12, 2-10, 2-8, 2-6, or 2-4 carbon atoms (e.g., C₂-C₂₀, C₂-C₁₈, C₂-C₁₆, C₂-C₁₄, C₂-C₁₂, C₂-C₁₀, C₂-C₈, C₂-C₆, or C₂-C₄). Examples include, but are not limited to, methyl, ethyl, isobutyl, sec-butyl, tert-butyl, 2-propenyl, and 3-butynyl.

The terms “alkylene,” “alkenylene,” and “alkynylene,” as used herein, refer to divalent groups having a specified size. In some embodiments, an alkylene may contain, e.g., 1-20, 1-18, 1-16, 1-14, 1-12, 1-10, 1-8, 1-6, 1-4, or 1-2 carbon atoms (e.g., C₁-C₂₀, C₁-C₁₈, C₁-C₁₆, C₁-C₁₄, C₁-C₁₂, C₁-C₁₀, C₁-C₈, C₁-C₆, C₁-C₄, or C₁-C₂). In some embodiments, an alkenylene or alkynylene may contain, e.g., 2-20, 2-18, 2-16, 2-14, 2-12, 2-10, 2-8, 2-6, or 2-4 carbon atoms (e.g., C₂-C₂₀, C₂-C₁₈, C₂-C₁₆, C₂-C₁₄, C₂-C₁₂, C₂-C₁₀, C₂-C₈, C₂-C₆, or C₂-C₄). Alkylene, alkenylene, and/or alkynylene includes straight-chain and branched-chain forms, as well as combinations of these. The divalency of an alkylene, alkenylene, or alkynylene group does not include the optional substituents on the alkylene, alkenylene, or alkynylene group. Each of the alkylene, alkenylene, and/or alkynylene groups in the linker is considered divalent with respect to the two attachments on either end of alkylene, alkenylene, and/or alkynylene group. For example, if a linker includes -(optionally substituted alkylene)-(optionally substituted alkenylene)-(optionally substituted alkylene)-, the alkenylene is considered divalent with respect to its attachments to the two alkylenes at the ends of the linker. The optional substituents on the alkenylene are not included in the divalency of the alkenylene. The divalent nature of an alkylene, alkenylene, or alkynylene group (e.g., an alkylene, alkenylene, or alkynylene group in a linker) refers to both of the ends of the group and does not include optional substituents that may be present in an alkylene, alkenylene, or alkynylene group. Because they are divalent, they can link together multiple (e.g., two) parts of a conjugate. Alkylene, alkenylene, and/or alkynylene groups can be substituted by the groups typically suitable as substituents for alkyl, alkenyl, and alkynyl groups as set forth herein. For example, C═O is a C1 alkylene that is substituted by an oxo (═O). For example, —HCR—C≡C— may be considered as an optionally substituted alkynylene and is considered a divalent group even though it has an optional substituent, R. Heteroalkylene, heteroalkenylene, and/or heteroalkynylene groups refer to alkylene, alkenylene, and/or alkynylene groups including one or more, e.g., 1-4, 1-3, 1, 2, 3, or 4, heteroatoms, e.g., N, O, and S. For example, a polyethylene glycol (PEG) polymer or a PEG unit —(CH₂)₂—O— in a PEG polymer is considered a heteroalkylene containing one or more oxygen atoms.

The term “amino,” as used herein, represents —N(R^{x})₂ or —N+(R^{x})₃, where each R^{x} is, independently, H, alkyl, alkenyl, alkynyl, aryl, alkaryl, carbocyclyl (e.g., cycloalkyl), or two R^{x} combine to form a heterocycloalkyl. In some embodiment, the amino group is —NH₂.

The term “aryl,” as used herein, refers to any monocyclic or fused polycyclic (e.g., bicyclic or tricyclic) ring system of carbon atoms which has the characteristics of aromaticity in terms of electron distribution throughout at least one (e.g., one, two, or three) ring of the ring system, e.g., phenyl, naphthyl, indanyl, 1H-indenyl, fluorenyl, or phenanthrenyl. In some embodiments, the ring system has the characteristics of aromaticity in terms of electron distribution throughout every ring of the ring system, e.g., phenyl, naphthyl, or phenanthrenyl. In some embodiments, a ring system contains 6-22 ring member atoms, 6-16 ring member atoms, 6-10 ring member atoms, 5-15 ring member atoms, or 5-10 ring member atoms. An aryl group may have, e.g., 5 to 22 carbons (e.g., a C₅-C₆, C₅-C₇, C₅-C₈, C₅-C₉, C₅-C₁₀, C₅-C₁₁, C₅-C₁₂, C₅-C₁₃, C₅-C₁₄, C₅-C₁₅, C₅-C₂₂, C₆-C₁₀, C₆-C₁₄, C₆-C₁₈, or C₆-C₂₂ aryl).

The term “heteroaryl” refers to a monocyclic or fused polycyclic (e.g., bicyclic or tricyclic) ring system which has the characteristics of aromaticity in terms of electron distribution through at least one (e.g., one, two, or three) ring of the ring system, where the ring system includes at least one aromatic ring containing one or more, e.g., 1-4, 1-3, 1, 2, 3, or 4, heteroatoms selected from O, S, and N, e.g., pyridyl, pyrimidyl, indolyl, isoindolyl, cinnolyl, phthalazyl, quinazolyl, quinoxalyl, benzofuranyl, benzothiophenyl, quinolyl, carbazolyl, benzimidazolyl, benzoxazolyl, benzothiazolyl, 1H-indazolyl, 1,2-benzisoxazolyl, 1,2-benzisothiazolyl, purinyl, dibenzofuranyl, acridinyl, phenazinyl, 5,6,7,8-tetrahydroquinolyl, or pyrindinyl. In some embodiments, the ring system has the characteristics of aromaticity in terms of electron distribution throughout every ring of the ring system, e.g., pyridyl, pyrimidyl, indolyl, isoindolyl, cinnolyl, phthalazyl, quinazolyl, quinoxalyl, benzofuranyl, benzothiophenyl, quinolyl, carbazolyl, benzimidazolyl, benzoxazolyl, benzothiazolyl, 1H-indazolyl, 1,2-benzisoxazolyl, 1,2-benzisothiazolyl, purinyl, dibenzofuranyl, acridinyl, phenazinyl. A heteroaryl group may have, e.g., 3 to 21 ring member atoms (e.g., a C₂-C₃, C₂-C₄, C₂-C₅, C₂-C₆, C₂-C₇, C₂-C₈, C₂-C₉, C₂-C₁₀, C₂-C₁₁, C₂-C₁₂, C₂-C₁₃, C₂-C₁₄, C₂-C₁₅, C₂-C₁₆, C₂-C₁₇, C₂-C₁₈, C₂-C₁₉, or C₂-C₂₀ heteroaryl). The inclusion of a heteroatom permits inclusion of 5-membered rings to be considered aromatic as well as 6-membered rings. Thus, typical heteroaryl systems include, e.g., pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, triazolyl (e.g., 1,2,3- or 1,2,4-triazolyl) oxazolyl, isoxazolyl, benzoxazolyl, benzoisoxazolyl, and imidazolyl. One or two ring carbon atoms of the heteroaryl group may be replaced with a carbonyl group (e.g., because tautomers are possible, a group such as phthalimido is also considered heteroaryl). In some embodiments, the aryl or heteroaryl group is a 5- or 6-membered aromatic rings system optionally containing 1-2 nitrogen atoms. In some embodiments, the aryl or heteroaryl group is an optionally substituted phenyl, pyridyl, indolyl, pyrimidyl, pyridazinyl, benzothiazolyl, benzimidazolyl, pyrazolyl, imidazolyl, isoxazolyl, thiazolyl, or imidazopyridinyl. In some embodiments, the aryl group is phenyl. In some embodiments, an aryl group may be optionally substituted with a substituent such an aryl substituent, e.g., biphenyl.

The term “arylene,” as used herein, refers to a multivalent (e.g., divalent or trivalent) aryl group linking together multiple (e.g., two or three) parts of a compound. For example, one carbon within the arylene group may be linked to one part of the compound, while another carbon within the arylene group may be linked to another part of the compound. An arylene may have, e.g., 5 to 22 carbons in the aryl portion of the arylene (e.g., a C₅-C₆, C₅-C₇, C₅-C₈, C₅-C₉, C₅-C₁₀, C₅-C₁, C₅-C₁₂, C₅-C₁₃, C₅-C₁₄, C₅-C₁₅, C₅-C₂₂, C₆-C₁₀, C₆-C₁₄, C₆-C₁₈, or C₆-C₂₂ arylene). An arylene group can be substituted by the groups typically suitable as substituents for alkyl, alkenyl, and alkynyl groups as set forth herein.

The term “heteroarylene,” as used herein, refers to a multivalent (e.g., divalent or trivalent) heteroaryl group linking together multiple (e.g., two or three) parts of a compound. A heteroarylene group may have, e.g., 3 to 21 ring member atoms having, e.g., 2 to 20 carbons (e.g., a C₂-C₃, C₂-C₄, C₂-C₅, C₂-C₆, C₂-C₇, C₂-C₈, C₂-C₉, C₂-C₁₀, C₂-C₁₁, C₂-C₁₂, C₂-C₁₃, C₂-C₁₄, C₂-C₁₅, C₂-C₁₆, C₂-C₁₇, C₂-C₁₈, C₂-C₁₉, or C₂-C₂₀ heteroarylene).

The term “carbocyclyl,” as used herein, represents a monocyclic or polycyclic (e.g., bicyclic or tricyclic) non-aromatic ring system in which the rings are formed by carbon atoms. A carbocyclyl group may have, e.g., 3 to 20 ring member atoms (e.g., C₃-C₄, C₃-C₅, C₃-C₆, C₃-C₇, C₃-C₈, C₃-C₉, C₃-C₁₀, C₃-C₁₁, C₃-C₁₂, C₃-C₁₃, C₃-C₁₄, C₃-C₁₅, C₃-C₁₆, C₃-C₁₇, C₃-C₁₈, C₃-C₁₉, or C₃-C₂₀ carbocyclyl). Examples of carbocyclyl groups include, but are not limited to, cycloalkyl (e.g., cyclohexyl), cycloalkenyl (e.g., cyclohexenyl), and cycloalkynyl (e.g., cyclooctyne). The term “cycloalkyl,” as used herein, represents a monovalent saturated cyclic alkyl group. A cycloalkyl may have, e.g., three to twenty carbons (e.g., a C₃-C₇, C₃-C₈, C₃-C₉, C₃-C₁₀, C₃-C₁₁, C₃-C₁₂, C₃-C₁₄, C₃-C₁₆, C₃-C₁₈, or C₃-C₂₀ cycloalkyl). Examples of cycloalkyls include, but are not limited to, cyclopropyl, cyclobutyl, cyclopentyl, cyclohexyl, and cycloheptyl. When the cycloalkyl group includes at least one carbon-carbon double bond, the cycloalkyl group can be referred to as a “cycloalkenyl” group. A cycloalkenyl may have, e.g., four to twenty carbons (e.g., a C₄-C₇, C₄-C₈, C₄-C₉, C₄-C₁₀, C₄-C₁₁, C₄-C₁₂, C₄-C₁₄, C₄-C₁₆, C₄-C₁₈, or C₄-C₂₀ cycloalkenyl). Exemplary cycloalkenyl groups include, but are not limited to, cyclopentenyl, cyclohexenyl, and cycloheptenyl. When the cycloalkyl group includes at least one carbon-carbon triple bond, the cycloalkyl group can be referred to as a “cycloalkynyl” group. A cycloalkynyl may have, e.g., eight to twenty carbons (e.g., a C₈-C₉, C₈-C₁₀, C₈-C₁₁, C₈-C₁₂, C₈-C₁₄, C₈-C₁₆, C₈-C₁₈, or C₈-C₂₀ cycloalkynyl). The term “cycloalkyl” also includes a cyclic compound having a bridged multicyclic structure in which one or more carbons bridges two non-adjacent members of a monocyclic ring, e.g., bicyclo[2.2.1.]heptyl and adamantane. The term “cycloalkyl” also includes bicyclic, tricyclic, and tetracyclic fused ring structures, e.g., decalin and spiro cyclic compounds.

A “heterocyclyl” refers to a monocyclic or polycyclic (e.g., bicyclic or tricyclic) ring system having at least one non-aromatic ring containing 1, 2, 3, or 4 ring atoms selected from N, O, or S, and no aromatic ring containing any N, O, or S atoms. A heterocyclyl group may have, e.g., 3 to 21 ring member atoms having, e.g., 2 to 20 carbons (e.g., C₂-C₃, C₂-C₄, C₂-C₅, C₂-C₆, C₂-C₇, C₂-C₈, C₂-C₉, C₂-C₁₀, C₂-C₁₁, C₂-C₁₂, C₂-C₁₃, C₂-C₁₄, C₂-C₁₅, C₂-C₁₆, C₂-C₁₇, C₂-C₁₈, C₂-C₁₉, or C₂-C₂₀ heterocyclyl). Examples of heterocyclyl groups include, but are not limited to, heterocycloalkyl, heterocycloalkenyl, and heterocycloalkynyl. A “heterocycloalkyl,” “heterocycloalkenyl,” or “heterocycloalkynyl” group refers to a cycloalkyl, cycloalkenyl, or cycloalkynyl group having one or more rings (e.g., 1, 2, 3, 4 or more rings) that has one or more heteroatoms independently selected from, e.g., N, O, and S. Exemplary heterocycloalkyl groups include pyrrolidinyl, thiolanyl, tetrahydrofuranyl, piperidinyl, tetrahydropyranyl, pyrrolizidinyl, and phenoxazinyl.

The term “carbocyclylene,” as used herein, refers to a multivalent (e.g., divalent or trivalent) carbocyclyl group linking together multiple (e.g., two or three) parts of a compound. For example, one carbon within the cycloalkylene group may be linked to one part of the compound, while another carbon within the cycloalkylene group may be linked to another part of the compound. A carbocyclylene may have, e.g., three to twenty carbons in the cyclic portion of the carbocyclylene (e.g., a C₃-C₇, C₃-C₈, C₃-C₉, C₃-C₁₀, C₃-C₁₁, C₃-C₁₂, C₃-C₁₄, C₃-C₁₆, C₃-C₁₈, or C₃-C₂₀ carbocyclylene). The term “cycloalkylene” refers to a multivalent (e.g., divalent or trivalent) cycloalkyl group linking together multiple (e.g., two or three) parts of a compound. When the cycloalkylene group includes at least one carbon-carbon double bond, the cycloalkylene group can be referred to as a “cycloalkenylene” group. A cycloalkenylene may have, e.g., four to twenty carbons in the cyclic portion of the cycloalkenylene (e.g., a C₄-C₇, C₄-C₈, C₄-C₉. C₄-C₁₀, C₄-C₁₁, C₄-C₁₂, C₄-C₁₄, C₄-C₁₆, C₄-C₁₈, or C₄-C₂₀ cycloalkenylene). When the cycloalkylene group includes at least one carbon-carbon triple bond, the cycloalkylene group can be referred to as a “cycloalkynylene” group. A cycloalkynylene may have, e.g., four to twenty carbons in the cyclic portion of the cycloalkynylene (e.g., a C₄-C₇, C₄-C₈, C₄-C₉, C₄-C₁₀, C₄-C₁₁, C₄-C₁₂, C₄-C₁₄, C₄-C₁₆, C₄-C₁₈, or C₈-C₂₀ cycloalkynylene). A carbocyclylene group (e.g., cycloalkylene, cycloalkenylene, and cycloalkynylene group) can be substituted by the groups typically suitable as substituents for alkyl, alkenyl, and alkynyl groups as set forth herein. Examples of cycloalkylene include, but are not limited to, cyclopropylene and cyclobutylene.

A “heterocyclylene” is a multivalent (e.g., divalent or trivalent) heterocyclyl group linking together multiple (e.g., two or three) parts of a compound. For example, one atom within the heterocyclylene group may be linked to one part of the compound, while another atom within the heterocyclylene group may be linked to another part of the compound. A heterocyclylene may have, e.g., 3 to 21 ring member atoms having, e.g., 2 to 20 carbons (e.g., C₂-C₃, C₂-C₄, C₂-C₅, C₂-C₆, C₂-C₇, C₂-C₈, C₂-C₉, C₂-C₁₀, C₂-C₁₁, C₂-C₁₂, C₂-C₁₃, C₂-C₁₄, C₂-C₁₅, C₂-C₁₆, C₂-C₁₇, C₂-C₁₈, C₂-C₁₉, or C₂-C₂₀ heterocyclylene). The term “heterocycloalkyl” refers to a multivalent (e.g., divalent or trivalent) heterocycloalkyl group linking together multiple (e.g., two or three) parts of a compound. When the heterocycloalkylene group includes at least one carbon-carbon double bond, the heterocycloalkylene group can be referred to as a “heterocycloalkenylene” group. A heterocycloalkenylene may have, e.g., four to twenty carbons in the cyclic portion of the heterocycloalkenylene (e.g., a C₄-C₇, C₄-C₈, C₄-C₉. C₄-C₁₀, C₄-C₁₁, C₄-C₁₂, C₄-C₁₄, C₄-C₁₆, C₄-C₁₈, or C₄-C₂₀ heterocycloalkenylene). When the heterocycloalkylene group includes at least one carbon-carbon triple bond, the heterocycloalkylene group can be referred to as a “heterocycloalkynylene” group. A heterocycloalkynylene may have, e.g., four to twenty carbons in the cyclic portion of the heterocycloalkynylene (e.g., a C₄-C₇, C₄-C₈, C₄-C₉, C₄-C₁₀, C₄-C₁₁, C₄-C₁₂, C₄-C₁₄, C₄-C₁₆, C₄-C₁₈, or C₈-C₂₀ heterocycloalkynylene). A heterocyclylene group (e.g., heterocycloalkylene, heterocycloalkenylene, hetero and cycloalkynylene group) can be substituted by the groups typically suitable as substituents for alkyl, alkenyl, and alkynyl groups as set forth herein.

The term “alkaryl,” refers to an aryl group that is connected to an alkylene, alkenylene, or alkynylene group. In general, if a compound is attached to an alkaryl group, the alkylene, alkenylene, or alkynylene portion of the alkaryl is attached to the compound. In some embodiments, an alkaryl is C₆-C₃₅ alkaryl (e.g., C₆-C₁₆, C₆-C₁₄, C₆-C₁₂, C₆-C₁₀, C₆-C₉, C₆-C₈, C₇, or C₆ alkaryl), in which the number of carbons indicates the total number of carbons in both the aryl portion and the alkylene, alkenylene, or alkynylene portion of the alkaryl. Examples of alkaryls include, but are not limited to, (C₁-C₈)alkylene(C₆-C₁₂)aryl, (C₂-C₈)alkenylene(C₆-C₁₂)aryl, or (C₂-C₈)alkynylene(C₆-C₁₂)aryl. In some embodiments, an alkaryl is benzyl or phenethyl. In a heteroalkaryl, one or more heteroatoms selected from N, O, and S may be present in the alkylene, alkenylene, or alkynylene portion of the alkaryl group and/or may be present in the aryl portion of the alkaryl group. In an optionally substituted alkaryl, the substituent may be present on the alkylene, alkenylene, or alkynylene portion of the alkaryl group and/or may be present on the aryl portion of the alkaryl group.

The term “alkamino,” as used herein, refers to an amino group, described herein, that is attached to an alkylene (e.g., C₁-C₅ alkylene), alkenylene (e.g., C₂-C₅ alkenylene), or alkynylene group (e.g., C₂-C₅ alkenylene). In general, if a compound is attached to an alkamino group, the alkylene, alkenylene, or alkynylene portion of the alkamino is attached to the compound. The amino portion of an alkamino refers to —N(R^{x})₂ or —N+(R^{x})₃, where each R^{x} is, independently, H, alkyl, alkenyl, alkynyl, aryl, alkaryl, cycloalkyl, or two R^{x} combine to form a heterocycloalkyl. In some embodiment, the amino portion of an alkamino is —NH₂. An example of an alkamino group is C₁-C₅ alkamino, e.g., C₂ alkamino (e.g., CH₂CH₂NH₂ or CH₂CH₂N(CH₃)₂). In a heteroalkamino group, one or more, e.g., 1-4, 1-3, 1, 2, 3, or 4, heteroatoms selected from N, O, and S may be present in the alkylene, alkenylene, or alkynylene portion of the heteroalkamino group. In some embodiments, an alkamino group may be optionally substituted. In a substituted alkamino group, the substituent may be present on the alkylene, alkenylene, or alkynylene portion of the alkamino group and/or may be present on the amino portion of the alkamino group.

The term “alkamide,” as used herein, refers to an amide group that is attached to an alkylene (e.g., C₁-C₅ alkylene), alkenylene (e.g., C₂-C₅ alkenylene), or alkynylene (e.g., C₂-C₅ alkenylene) group. In general, if a compound is attached to an alkamide group, the alkylene, alkenylene, or alkynylene portion of the alkamide is attached to the compound. The amide portion of an alkamide refers to —C(O)—N(R^{x})₂, where each R^{x} is, independently, H, alkyl, alkenyl, alkynyl, aryl, alkaryl, cycloalkyl, or two R^{x} combine to form a heterocycloalkyl. In some embodiment, the amide portion of an alkamide is —C(O)NH₂. An alkamide group may be —(CH₂)₂—C(O)NH₂ or —CH₂—C(O)NH₂. In a heteroalkamide group, one or more, e.g., 1-4, 1-3, 1, 2, 3, or 4, heteroatoms selected from N, O, and S may be present in the alkylene, alkenylene, or alkynylene portion of the heteroalkamide group. In some embodiments, an alkamide group may be optionally substituted. In a substituted alkamide group, the substituent may be present on the alkylene, alkenylene, or alkynylene portion of the alkamide group and/or may be present on the amide portion of the alkamide group.

The term “azido,” as used herein, refers to a group having the structure:

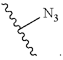 [SMILES: *N=[N+]=[N-]]

The term “carbonyl,” as used herein, refers to a group having the structure:

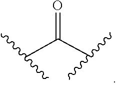 [SMILES: *C(*)=O]

The term “cyano,” as used herein, refers to a group having the structure:

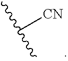 [SMILES: *C#N]

The terms “halo” or “halogen,” as used herein, refer to a fluorine (fluoro), chlorine (chloro), bromine (bromo), or iodine (iodo) radical.

The term “haloalkyl,” as used herein, refers to an alkyl group substituted with one or more (e.g., one, two, three, four, five, six, or more) halo groups. Haloalkyl groups include, but are not limited to, fluoroalkyl (e.g., trifluoromethyl and pentafluoroethyl) and chloroalkyl.

The term “hydroxyl,” as used herein, represents an —OH group.

The term “imino,” as used herein, represents the group having the structure:

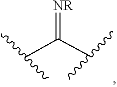

wherein R is an optional substituent.

The term “nitro,” as used herein, refers to a group having the structure:

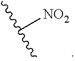 [SMILES: *[N+](=O)[O-]]

The term “N-protecting group,” as used herein, represents those groups intended to protect an amino group against undesirable reactions during synthetic procedures. Commonly used N-protecting groups are disclosed in Greene, “Protective Groups in Organic Synthesis,” 5th Edition (John Wiley & Sons, New York, 2014), which is incorporated herein by reference. N-protecting groups include, e.g., acyl, aryloyl, and carbamyl groups such as formyl, acetyl, propionyl, pivaloyl, t-butylacetyl, 2-chloroacetyl, 2-bromoacetyl, trifluoroacetyl, trichloroacetyl, phthaloyl, o-nitrophenoxyacetyl, α-chlorobutyryl, benzoyl, carboxybenzyl (CBz), 4-chlorobenzoyl, 4-bromobenzoyl, 4-nitrobenzoyl, and chiral auxiliaries such as protected or unprotected D, L or D, L-amino acid residues such as alanine, leucine, phenylalanine; sulfonyl-containing groups such as benzenesulfonyl and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl (BOC), diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl (Fmoc), cyclopentyloxycarbonyl, adamantyloxycarbonyl, cyclohexyloxycarbonyl, and phenylthiocarbonyl; alkaryl groups such as benzyl, triphenylmethyl, and benzyloxymethyl; and silyl groups such as trimethylsilyl.

The term “oxo,” as used herein, refers to a substituent having the structure ═O, where there is a double bond between an atom and an oxygen atom.

The term “phosphate,” as used herein, represents the group having the structure:

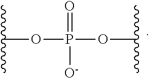 [SMILES: *OP(=O)([O-])O*]

The term “phosphoryl,” as used herein, represents the group having the structure:

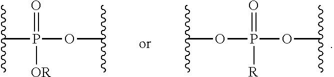

The term “sulfonyl,” as used herein, represents the group having the structure:

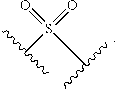 [SMILES: *S(*)(=O)=O]

The term “thiocarbonyl,” as used herein, refers to a group having the structure:

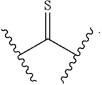 [SMILES: *C(*)=S]

The term “amino acid,” as used herein, means naturally occurring amino acids and non-naturally occurring amino acids.

The term “naturally occurring amino acids,” as used herein, means amino acids including Ala, Arg, Asn, Asp, Cys, Gln, Glu, Gly, His, lie, Leu, Lys, Met, Phe, Pro, Ser, Thr, Trp, Tyr, and Val.

The term “non-naturally occurring amino acid,” as used herein, means an alpha amino acid that is not naturally produced or found in a mammal. Examples of non-naturally occurring amino acids include D-amino acids; an amino acid having an acetylaminomethyl group attached to a sulfur atom of a cysteine; a pegylated amino acid; the omega amino acids of the formula NH₂(CH₂)ₙCOOH where n is 2-6, neutral nonpolar amino acids, such as sarcosine, t-butyl alanine, t-butyl glycine, N-methyl isoleucine, and norleucine; oxymethionine; phenylglycine; citrulline; methionine sulfoxide; cysteic acid; ornithine; diaminobutyric acid; 3-aminoalanine; 3-hydroxy-D-proline; 2,4-diaminobutyric acid; 2-aminopentanoic acid; 2-aminooctanoic acid, 2-carboxy piperazine; piperazine-2-carboxylic acid, 2-amino-4-phenylbutanoic acid; 3-(2-naphthyl)alanine, and hydroxyproline. Other amino acids are α-aminobutyric acid, α-amino-α-methylbutyrate, aminocyclopropane-carboxylate, aminoisobutyric acid, aminonorbornyl-carboxylate, L-cyclohexylalanine, cyclopentylalanine, L-N-methylleucine, L-N-methylmethionine, L-N-methylnorvaline, L-N-methylphenylalanine, L-N-methylproline, L-N-methylserine, L-N-methyltryptophan, D-ornithine, L-N-methylethylglycine, L-norleucine, α-methyl-aminoisobutyrate, α-methylcyclohexylalanine, D-α-methylalanine, D-α-methylarginine, D-α-methylasparagine, D-α-methylaspartate, D-α-methylcysteine, D-α-methylglutamine, D-α-methylhistidine, D-α-methylisoleucine, D-α-methylleucine, D-α-methyllysine, D-α-methylmethionine, D-α-methylornithine, D-α-methylphenylalanine, D-α-methylproline, D-α-methylserine, D-N-methylserine, D-α-methylthreonine, D-α-methyltryptophan, D-α-methyltyrosine, D-α-methylvaline, D-N-methylalanine, D-N-methylarginine, D-N-methylasparagine, D-N-methylaspartate, D-N-methylcysteine, D-N-methylglutamine, D-N-methylglutamate, D-N-methylhistidine, D-N-methylisoleucine, D-N-methylleucine, D-N-methyllysine, N-methylcyclohexylalanine, D-N-methylornithine, N-methylglycine, N-methylaminoisobutyrate, N-(1-methylpropyl)glycine, N-(2-methylpropyl)glycine, D-N-methyltryptophan, D-N-methyltyrosine, D-N-methylvaline, γ-aminobutyric acid, L-t-butylglycine, L-ethylglycine, L-homophenylalanine, L-α-methylarginine, L-α-methylaspartate, L-α-methylcysteine, L-α-methylglutamine, L-α-methylhistidine, L-α-methylisoleucine, L-α-methylleucine, L-α-methylmethionine, L-α-methylnorvaline, L-α-methylphenylalanine, L-α-methylserine, L-α-methyltryptophan, L-α-methylvaline, N-(N-(2,2-diphenylethyl) carbamylmethylglycine, 1-carboxy-1-(2,2-diphenyl-ethylamino) cyclopropane, 4-hydroxyproline, ornithine, 2-aminobenzoyl (anthraniloyl), D-cyclohexylalanine, 4-phenyl-phenylalanine, L-citrulline, α-cyclohexylglycine, L-1,2,3,4-tetrahydroisoquinoline-3-carboxylic acid, L-thiazolidine-4-carboxylic acid, L-homotyrosine, L-2-furylalanine, L-histidine (3-methyl), N-(3-guanidinopropyl)glycine, O-methyl-L-tyrosine, O-glycan-serine, meta-tyrosine, nor-tyrosine, L-N,N′,N″-trimethyllysine, homolysine, norlysine, N-glycan asparagine, 7-hydroxy-1,2,3,4-tetrahydro-4-fluorophenylalanine, 4-methylphenylalanine, bis-(2-picolyl)amine, pentafluorophenylalanine, indoline-2-carboxylic acid, 2-aminobenzoic acid, 3-amino-2-naphthoic acid, asymmetric dimethylarginine, L-tetrahydroisoquinoline-1-carboxylic acid, D-tetrahydroisoquinoline-1-carboxylic acid, 1-amino-cyclohexane acetic acid, D/L-allylglycine, 4-aminobenzoic acid, 1-amino-cyclobutane carboxylic acid, 2 or 3 or 4-aminocyclohexane carboxylic acid, 1-amino-1-cyclopentane carboxylic acid, 1-aminoindane-1-carboxylic acid, 4-amino-pyrrolidine-2-carboxylic acid, 2-aminotetraline-2-carboxylic acid, azetidine-3-carboxylic acid, 4-benzyl-pyrolidine-2-carboxylic acid, tert-butylglycine, b-(benzothiazolyl-2-yl)-alanine, b-cyclopropyl alanine, 5,5-dimethyl-1,3-thiazolidine-4-carboxylic acid, (2R,4S)4-hydroxypiperidine-2-carboxylic acid, (2S,4S) and (2S,4R)-4-(2-naphthylmethoxy)-pyrolidine-2-carboxylic acid, (2S,4S) and (2S,4R)4-phenoxy-pyrrolidine-2-carboxylic acid, (2R,5S) and (2S,5R)-5-phenyl-pyrrolidine-2-carboxylic acid, (2S,4S)-4-amino-1-benzoyl-pyrrolidine-2-carboxylic acid, t-butylalanine, (2S,5R)-5-phenyl-pyrrolidine-2-carboxylic acid, 1-aminomethyl-cyclohexane-acetic acid, 3,5-bis-(2-amino)ethoxy-benzoic acid, 3,5-diamino-benzoic acid, 2-methylamino-benzoic acid, N-methylanthranylic acid, L-N-methylalanine, L-N-methylarginine, L-N-methylasparagine, L-N-methylaspartic acid, L-N-methylcysteine, L-N-methylglutamine, L-N-methylglutamic acid, L-N-methylhistidine, L-N-methylisoleucine, L-N-methyllysine, L-N-methylnorleucine, L-N-methylornithine, L-N-methylthreonine, L-N-methyltyrosine, L-N-methylvaline, L-N-methyl-t-butylglycine, L-norvaline, α-methyl-γ-aminobutyrate, 4,4′-biphenylalanine, α-methylcylcopentylalanine, α-methyl-α-napthylalanine, α-methylpenicillamine, N-(4-aminobutyl)glycine, N-(2-aminoethyl)glycine, N-(3-aminopropyl)glycine, N-amino-α-methylbutyrate, α-napthylalanine, N-benzylglycine, N-(2-carbamylethyl)glycine, N-(carbamylmethyl)glycine, N-(2-carboxyethyl)glycine, N-(carboxymethyl)glycine, N-cyclobutylglycine, N-cyclodecylglycine, N-cycloheptylglycine, N-cyclohexylglycine, N-cyclodecylglycine, N-cylcododecylglycine, N-cyclooctylglycine, N-cyclopropylglycine, N-cycloundecylglycine, N-(2,2-diphenylethyl)glycine, N-(3,3-diphenylpropyl)glycine, N-(3-guanidinopropyl)glycine, N-(1-hydroxyethyl)glycine, N-(hydroxyethyl))glycine, N-(imidazolylethyl))glycine, N-(3-indolylyethyl)glycine, N-methyl-γ-aminobutyrate, D-N-methylmethionine, N-methylcyclopentylalanine, D-N-methylphenylalanine, D-N-methylproline, D-N-methylthreonine, N-(1-methylethyl)glycine, N-methyl-napthylalanine, N-methylpenicillamine, N-(p-hydroxyphenyl)glycine, N-(thiomethyl)glycine, penicillamine, L-α-methylalanine, L-α-methylasparagine, L-α-methyl-t-butylglycine, L-methylethylglycine, L-α-methylglutamate, L-α-methylhomophenylalanine, N-(2-methylthioethyl)glycine, L-α-methyllysine, L-α-methylnorleucine, L-α-methylornithine, L-α-methylproline, L-α-methylthreonine, L-α-methyltyrosine, L-N-methyl-homophenylalanine, N-(N-(3,3-diphenylpropyl) carbamylmethylglycine, L-pyroglutamic acid, D-pyroglutamic acid, O-methyl-L-serine, O-methyl-L-homoserine, 5-hydroxylysine, α-carboxyglutamate, phenylglycine, L-pipecolic acid (homoproline), L-homoleucine, L-lysine (dimethyl), L-2-naphthylalanine, L-dimethyldopa or L-dimethoxy-phenylalanine, L-3-pyridylalanine, L-histidine (benzoyloxymethyl), N-cycloheptylglycine, L-diphenylalanine, O-methyl-L-homotyrosine, L-p-homolysine, O-glycan-threoine, Ortho-tyrosine, L-N,N′-dimethyllysine, L-homoarginine, neotryptophan, 3-benzothienylalanine, isoquinoline-3-carboxylic acid, diaminopropionic acid, homocysteine, 3,4-dimethoxyphenylalanine, 4-chlorophenylalanine, L-1,2,3,4-tetrahydronorharman-3-carboxylic acid, adamantylalanine, symmetrical dimethylarginine, 3-carboxythiomorpholine, D-1,2,3,4-tetrahydronorharman-3-carboxylic acid, 3-aminobenzoic acid, 3-amino-1-carboxymethyl-pyridin-2-one, 1-amino-1-cyclohexane carboxylic acid, 2-aminocyclopentane carboxylic acid, 1-amino-1-cyclopropane carboxylic acid, 2-aminoindane-2-carboxylic acid, 4-amino-tetrahydrothiopyran-4-carboxylic acid, azetidine-2-carboxylic acid, b-(benzothiazol-2-yl)-alanine, neopentylglycine, 2-carboxymethyl piperidine, b-cyclobutyl alanine, allylglycine, diaminopropionic acid, homo-cyclohexyl alanine, (2S,4R)-4-hydroxypiperidine-2-carboxylic acid, octahydroindole-2-carboxylic acid, (2S,4R) and (2S,4R)-4-(2-naphthyl), pyrrolidine-2-carboxylic acid, nipecotic acid, (2S,4R) and (2S,4S)-4-(4-phenylbenzyl) pyrrolidine-2-carboxylic acid, (3S)-1-pyrrolidine-3-carboxylic acid, (2S,4S)-4-tritylmercapto-pyrrolidine-2-carboxylic acid, (2S,4S)-4-mercaptoproline, t-butylglycine, N,N-bis(3-aminopropyl)glycine, 1-amino-cyclohexane-1-carboxylic acid, N-mercaptoethylglycine, and selenocysteine. In some embodiments, amino acid residues may be charged or polar. Charged amino acids include alanine, lysine, aspartic acid, or glutamic acid, or non-naturally occurring analogs thereof. Polar amino acids include glutamine, asparagine, histidine, serine, threonine, tyrosine, methionine, or tryptophan, or non-naturally occurring analogs thereof. It is specifically contemplated that in some embodiments, a terminal amino group in the amino acid may be an amido group or a carbamate group.

The term “pharmaceutically acceptable salt,” as used herein, represents salts of the conjugates described herein (e.g., conjugates of formula (M-I) or (M-II)) that are, within the scope of sound medical judgment, suitable for use in methods described herein without undue toxicity, irritation, and/or allergic response. Pharmaceutically acceptable salts are well known in the art. For example, pharmaceutically acceptable salts are described in: Pharmaceutical Salts: Properties, Selection, and Use (Eds. P. H. Stahl and C. G. Wermuth), Wiley-VCH, 2008. The salts can be prepared in situ during the final isolation and purification of the conjugates described herein or separately by reacting the free base group with a suitable organic acid.

Other features and advantages of the invention will be apparent from the following detailed description and the claims.

### DETAILED DESCRIPTION

Provided herein are methods for synthesizing protein-drug conjugates useful for the treatment diseases and related conditions. The conjugates disclosed herein (e.g., a conjugate of formula (M-I) or (M-II)) include a polypeptide, E (e.g., Fc domain monomer, an Fc domain, an Fc-binding peptide, an albumin protein, or an albumin protein-binding peptide), and a therapeutic agent, A¹. The compounds (e.g., a compound of formula (F-I) or (F-II)) and methods described herein are valuable in generating conjugates useful for the treatment of diseases and conditions thereof.

The methods disclosed herein can provide a number of advantages, such as higher overall yield and higher purity (e.g., efficient elimination of impurities) of the final product (e.g., a conjugate of formula (M-I) or (M-II)), as well as reduced waste stream (e.g., reducing the total number of reaction steps or reducing loss of starting material (e.g., polypeptide, E, and/or compound of formula (F-I) or (F-II)) and mild reaction conditions (e.g., step (c) or step (e) of the methods described herein). The methods of the disclosure can also enable reliable synthesis of the final product (e.g., a conjugate of formula (M-I) or (M-II)) having preferred characteristics, e.g., drug-to-antibody ratio (DAR).

### I. Therapeutic Agents of the Protein-Drug Conjugates

The protein-drug conjugates disclosed herein include a protein conjugated to one or more therapeutic agents (e.g., small molecules or biologics such as peptides, polypeptides, and polynucleotides) through one or more linkers (e.g.,

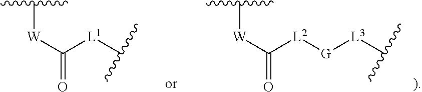

In some embodiments, when T is greater than 1 (e.g., T is 2, 3, 4, 5, 6, 7, 8, 9, 10, 11, 12, 13, 14, 15, 16, 17, 18, 19, or 20), each of

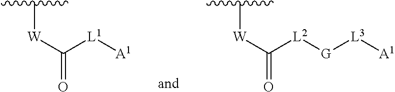

may be independently selected (e.g., independently selected from therapeutic agents and linkers described in WO 2020/051498, WO 2020/252393, WO 2020/252396, WO 2021/046549, or WO 2021/050612, each of which is hereby incorporated by reference).

In some embodiments, when T is greater than 1 (e.g., T is 2, 3, 4, 5, 6, 7, 8, 9, 10, 11, 12, 13, 14, 15, 16, 17, 18, 19, or 20), each therapeutic agent, A¹, may be independently selected (e.g., independently selected from therapeutic agents described in WO 2020/051498, WO 2020/252393, WO 2020/252396, WO 2021/046549, or WO 2021/050612).

In some embodiments, E may be conjugated to 2, 3, 4, 5, 6, 7, 8, 9, 10, or more different therapeutic agents. In some embodiments, E is conjugated to a first therapeutic agent, and a second therapeutic agent. In some embodiments, each A₁ the first therapeutic agent and of the second therapeutic agent are independently selected from any structure described in WO 2020/051498, WO 2020/252393, WO 2020/252396, WO 2021/046549, or WO 2021/050612.

In some embodiments, the therapeutic agent includes a monomer, e.g., of a small molecule. In some embodiments, the therapeutic agent includes a dimer, e.g., of small molecules. In some embodiments, the therapeutic agent includes a monomer or dimer by way of a linker. In some embodiments, the therapeutic agent includes a monomer by way of a linker. In some embodiments, the therapeutic agent includes a dimer by way of a linker.

In some embodiments, the therapeutic agent is a small molecule antiviral agent, antibacterial agent, or antifungal agent.

In some embodiments, the therapeutic agent is a small molecule antiviral agent. Small molecule antiviral agents are known to those of skill in the art and include, for example, zanamivir, peramivir, temsavir, pimovidir, oseltamivir, laninamivir, CS-8958, amantadine, rimantadine, cyanovirin-N, a cap-dependent endonuclease inhibitor (e.g., baloxavir acid or baloxavir marboxil), a polymerase inhibitor (e.g., T-705), a PB2 inhibitor (e.g., JNJ-63623872), a conjugated sialidase (e.g., DAS181), a thiazolide (e.g., nitazoxanide), a COX inhibitor, or a PPAR agonist. In some embodiments, the antiviral agent is selected from vidarabine, acyclovir, gancyclovir, valgancyclovir, a nucleoside-analog reverse transcriptase inhibitor (e.g., AZT (Zidovudine), ddI (Didanosine), ddC (Zalcitabine), d4T (Stavudine), or 3TC (Lamivudine)), and a non-nucleoside reverse transcriptase inhibitor (e.g., (nevirapine or delavirdine), protease inhibitor (saquinavir, ritonavir, indinavir, or nelfinavir), ribavirin, or interferon). In some embodiments, the antiviral agent is selected from lopinavir, ritonavir, remdesivir, favilavir, and galidesivir, In some embodiments, the antiviral agent is zanamivir or an analog thereof. In some embodiments, the antiviral agent is peramivir or an analog thereof. In some embodiments, the antiviral agent is temsavir or an analog thereof.

In some embodiments, the therapeutic agent is a small molecule antibacterial agent. Small molecule antibacterial agents are known to those of skill in the art and include, for example, amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem/cilastatin, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin g, penicillin v, piperacillin, penicillin g, temocillin, ticarcillin, amoxicillin clavulanate, ampicillin/sulbactam, piperacillin/tazobactam, ticarcillin/clavulanate, bacitracin, colistin, polymyxin b, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilamide, sulfasalazine, sulfisoxazole, trimethoprim-sulfamethoxazole (tmp-smx), sulfonamidochrysoidine, demeclocycline, doxycycline, minocycline, oxytetracycline, tetracycline, clofazimine, dapsone, capreomycin, cycloserine, ethambutol (bs), ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, and trimethoprim.

In some embodiments, the therapeutic agent is a small molecule antifungal agent. Small molecule antifungal agents are known to those of skill in the art and include, for example, rezafungin, anidulafungin, caspofungin, micafungin, amphotericin B, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, triazoles, albaconazole, efinaconazole, epoxiconazole, fluconazole, isavuconazole, itraconazole, posaconazole, propiconazole, ravuconazole, terconazole, voriconazole, abafungin, amorolfin, butenafine, naftifine, terbinafine, ciclopirox, flucytosine, griseofulvin, tolnaftate, and undecylenic acid.

### II. Proteins of the Protein-Drug Conjugates: Fc Domain Monomers and Fc Domains

The protein-drug conjugates disclosed herein include a polypeptide, E (e.g., Fc domain monomer, an Fc domain, an Fc-binding peptide, an albumin protein, or an albumin protein-binding peptide) conjugated to one or more therapeutic agents through one or more linkers.

An Fc domain monomer includes a hinge domain, a C_{H}2 antibody constant domain, and a C_{H}3 antibody constant domain. The Fc domain monomer can be of immunoglobulin antibody isotype IgG, IgE, IgM, IgA, or IgD. The Fc domain monomer can also be of any immunoglobulin antibody isotype (e.g., IgG1, IgG2a, IgG2b, IgG3, or IgG4). The Fc domain monomer can be of any immunoglobulin antibody allotype (e.g., IGHG1*01 (i.e., G1m(za)), IGHG1*07 (i.e., G1m(zax)), IGHG1*04 (i.e., G1m(zav)), IGHG1*03 (G1m(f)), IGHG1*08 (i.e., G1m(fa)), IGHG2*01, IGHG2*06, IGHG2*02, IGHG3*01, IGHG3*05, IGHG3*10, IGHG3*04, IGHG3*09, IGHG3*11, IGHG3*12, IGHG3*06, IGHG3*07, IGHG3*08, IGHG3*13, IGHG3*03, IGHG3*14, IGHG3*15, IGHG3*16, IGHG3*17, IGHG3*18, IGHG3*19, IGHG2*04, IGHG4*01, IGHG4*03, or IGHG4*02) (as described in, for example, in Vidarsson et al. IgG subclasses and allotypes: from structure to effector function. Frontiers in Immunology. 5(520):1-17 (2014)). The Fc domain monomer can also be of any species, e.g., human, murine, or mouse. A dimer of Fc domain monomers is an Fc domain that can bind to an Fc receptor, which is a receptor located on the surface of leukocytes.

In some embodiments, an Fc domain monomer in the conjugates described herein may contain one or more amino acid substitutions, additions, and/or deletion relative to an Fc domain monomer having a sequence as described in WO 2020/051498, WO 2020/252393, WO 2020/252396, WO 2021/046549, or WO 2021/050612. In some embodiments, an Fc domain monomer in the conjugates described herein include a sequence as described in WO 2020/051498, WO 2020/252393, WO 2020/252396, WO 2021/046549, or WO 2021/050612. In some embodiments, an Fc domain monomer in the conjugates described herein is an Fc domain monomer described in WO 2020/051498, WO 2020/252393, WO 2020/252396, WO 2021/046549, or WO 2021/050612.

In some embodiments, an Fc domain monomer in the conjugates as described herein includes an additional moiety, e.g., an albumin-binding peptide, a purification peptide, or a signal sequence attached to the N- or C-terminus of the Fc domain monomer. In some embodiments, an Fc domain monomer in the conjugate does not contain any type of antibody variable region, e.g., V_{H}, V_{L}, a complementarity determining region (CDR), or a hypervariable region (HVR).

In some embodiments, an Fc domain monomer in the conjugates described herein may have a sequence that is at least 95% identical to a sequence described in WO 2020/051498, WO 2020/252393, WO 2020/252396, WO 2021/046549, or WO 2021/050612.

In some embodiments, an F domain monomer in the conjugates as described herein may include a C220S mutation. In some embodiments, an F domain monomer in the conjugates as described herein may include a K246X mutation, wherein X is not a Lys, most preferably wherein X is selected from Ser, Gly, Ala, Thr, Asn, Gln, Arg, His, Glu, or Asp. In some embodiments, an F domain monomer in the conjugates as described herein may include one or more mutations that enhance binding to an Fc receptor (e.g., the FcRn receptor), such as M252Y/S254T/T256E (“YTE”), V309D/Q311H/N434S (“DHS”), and/or M428L/N434S (“LS”), wherein the numbering is according to the EU index as in Kabat. In some embodiments, amino acid substitutions are relative to a wild-type Fc monomer amino acid sequence, e.g., wild-type human IgG1 or IgG2.

In some embodiments, an Fc domain monomer in the conjugates as described herein may have a sequence of any one of SEQ ID NOs: 1-5, wherein the numbering is according to the EU index as in Kabat.

In some embodiments, an Fc domain monomer in the conjugates as described herein may have a sequence of SEQ ID NO: 1 shown below.

SEQ ID NO: 1: mature human IgG1 Fc; X₁ (position 201) is Asn or absent; X₂ (position 220) is Cys or Ser; X₃ (position 246) is Lys, Ser, Gly, Ala, Thr, Asn, Gln, Arg, His, Glu, or Asp; X₄ (position 252) is Met or Tyr; X₅ (position 254) is Ser or Thr; X₆ (position 256) is Thr or Glu; X₇ (position 297) is Asn or Ala; X₈ (position 309) is Leu or Asp; X₉ (position 311) is Gln or His; X₁₀ (position 356) is Asp or Glu; and X₁₁ (position 358) is Leu or Met; X₁₂ (position 428) is Met or Leu; X₁₃ (position 434) is Asn or Ser; X₁₄ (position 447) is Lys or absent

X₁VNHKPSNTKVDKKVEPKSX₂DKTHTCPPCPAPELLGGPSVFLFPPX₃P
KDTLX₄IX₅RX₆PEVTCVVVDVSHEDPEVKFNWYVDGVEVHNAKTKPREE
QYX₇STYRVVSVLTVX₈HX₉DWLNGKEYKCKVSNKALPAPIEKTISKAKG
QPREPQVYTLPPSRX₁₀EX₁₁TKNQVSLTCLVKGFYPSDIAVEWESNGQP
ENNYKTTPPVLDSDGSFFLYSKLTVDKSRWQQGNVFSCSVX₁₂HEALH
X₁₃HYTQKSLSLSPGX₁₄

In some embodiments of SEQ ID NO: 1, X₁ is Asn. In some embodiments of SEQ ID NO: 1, X₁ is absent. In some embodiments of SEQ ID NO: 1, X₂ is Cys. In some embodiments of SEQ ID NO: 1, X₂ is Ser. In some embodiments of SEQ ID NO: 1, X₃ is Lys. In some embodiments of SEQ ID NO: 1, X₃ is selected from Ser, Gly, Ala, Thr, Asn, Gln, Arg, His, Glu, or Asp. In some embodiments of SEQ ID NO: 1, X₃ is Ser. In some embodiments of SEQ ID NO: 1, X₄ is Met, X₅ is Ser, and X₆ is Thr. In some embodiments of SEQ ID NO: 1, X₄ is Tyr, X₉ is Thr, and X₆ is Glu. In some embodiments of SEQ ID NO: 1, X₇ is Asn. In some embodiments of SEQ ID NO: 1, X₇ is Ala. In some embodiments of SEQ ID NO: 1, X₈ Leu, X₉ is Gln, and X₁₃ is Asn. In some embodiments of SEQ ID NO: 1, X₈ is Asp, X₉ is His, and X₁₃ is Ser. In some embodiments of SEQ ID NO: 1, X₁₀ is Glu and X₁₁ is Met. In some embodiments of SEQ ID NO: 1, X₁₀ is Asp and X₁₁ is Leu. In some embodiments of SEQ ID NO: 1, X₁₂ is Met and X₁₃ is Asn. In some embodiments of SEQ ID NO: 1, X₁₂ is Leu and X₁₃ is Ser. In some embodiments of SEQ ID NO: 1, X₁₄ is Lys. In some embodiments of SEQ ID NO: 1, X₁₄ is absent.

In some embodiments, an Fc domain monomer in the conjugates as described herein may have a sequence of SEQ ID NO: 2 shown below.

SEQ ID NO: 2: mature human IgG1 Fc, Cys to Ser
substitution (#), allotype G1m(f) (bold italics)
NVNHKPSNTKVDKKVEPKSS(#)DKTHTCPPCPAPELLGGPSVFLFPPK
PKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSR [symbol] E [symbol] TKNQVSLTCLVKGFYPSDIAVEWESNGQPENNYK
TTPPVLDSDGSFFLYSKLTVDKSRWQQGNVFSCSVMHEALHNHYTQKSL
SLSPG

In some embodiments, an Fc domain monomer in the conjugates as described herein may have a sequence of SEQ ID NO: 4 shown below.

SEQ ID NO: 3: mature human IgG1 Fc, Cys to Ser
substitution (#), allotype G1m(fa) (bold italics)
NVNHKPSNTKVDKKVEPKSS(#)DKTHTCPPCPAPELLGGPSVFLFPPK
PKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSR [symbol] E [symbol] TKNQVSLTCLVKGFYPSDIAVEWESNGQPENNYK
TTPPVLDSDGSFFLYSKLTVDKSRWQQGNVFSCSVMHEALHNHYTQKSL
SLSPG

In some embodiments, an Fc domain monomer in the conjugates as described herein may have a sequence of SEQ ID NO: 4 shown below.

SEQ ID NO: 4: mature human IgG1 Fc, Cys to Ser
substitution (#), YTE triple mutation (bold and
underlined), allotype G1m(f) (bold italics)
NVNHKPSNTKVDKKVEPKSS(#)DKTHTCPPCPAPELLGGPSVFLFPPK
PKDTLYITREPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSR [symbol] E [symbol] TKNQVSLTCLVKGFYPSDIAVEWESNGQPENNYK
TTPPVLDSDGSFFLYSKLTVDKSRWQQGNVFSCSVMHEALHNHYTQKSL
SLSPG

In some embodiments, an Fc domain monomer in the conjugates as described herein may have a sequence of SEQ ID NO: 5 shown below.

SEQ ID NO: 5: mature human IgG1 Fc, Cys to Ser
substitution (#), YTE triple mutation (bold and
underlined), allotype G1m(fa) (bold italics)
NVNHKPSNTKVDKKVEPKSS(#)DKTHTCPPCPAPELLGGPSVFLFPPK
PKDTLYITREPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSR [symbol] E [symbol] TKNQVSLTCLVKGFYPSDIAVEWESNGQPENNYK
TTPPVLDSDGSFFLYSKLTVDKSRWQQGNVFSCSVMHEALHNHYTQKSL
SLSPG

As defined herein, an Fc domain includes two Fc domain monomers that are dimerized by the interaction between the C_{H}3 antibody constant domains, as well as one or more disulfide bonds that form between the hinge domains of the two dimerizing Fc domain monomers. An Fc domain forms the minimum structure that binds to an Fc receptor, e.g., Fc-gamma receptors (i.e., Fcγ receptors (FcγR)), Fc-alpha receptors (i.e., Fcα receptors (FcαR)), Fc-epsilon receptors (i.e., Fcε receptors (FcεR)), and/or the neonatal Fc receptor (FcRn). In some embodiments, an Fc domain of the present invention binds to an Fcγ receptor (e.g., FcRn, FcγRI (CD64), FcγRIIa (CD32), FcγRIIb (CD32), FcγRIIIa (CD16a), FcγRIIIb (CD16b)), and/or FcγRIV and/or the neonatal Fc receptor (FcRn).

In some embodiments, the Fc domain monomer or Fc domain of the invention is an aglycosylated Fc domain monomer or Fc domain (e.g., an Fc domain monomer or an Fc domain that maintains engagement to an Fc receptor (e.g., FcRn). For example, the Fc domain is an aglycosylated IgG1 variants that maintains engagement to an Fc receptor (e.g., an IgG1 having an amino acid substitution at N297 and/or T299 of the glycosylation motif). Exemplary aglycosylated Fc domains and methods for making aglycosylated Fc domains are known in the art, for example, as described in Sazinsky S. L. et al., Aglycosylated immunoglobulin G1 variants productively engage activating Fc receptors, PNAS, 2008, 105(51):20167-20172, which is incorporated herein in its entirety.

In some embodiments, the Fc domain or Fc domain monomer of the invention is engineered to enhance binding to the neonatal Fc receptor (FcRn). For example, the Fc domain may include the triple mutation corresponding to M252Y/S254T/T256E (YTE). The Fc domain may include the double mutant corresponding to M428L/N434S (LS). The Fc domain may include the triple mutant corresponding to V309D/Q311H/N434S (DHS). The Fc domain may include the single mutant corresponding to N434H (e.g., an IgG1, such as a human or humanized IgG1 having an N434H mutation). The Fc domain may include the single mutant corresponding to C220S. The Fc domain may include a combination of one or more of the above-described mutations that enhance binding to the FcRn. Enhanced binding to the FcRn may increase the half-life Fc domain-containing conjugate. For example, incorporation of one or more amino acid mutations that increase binding to the FcRn (e.g., a YTE mutation, an LS mutation, or an N434H mutation) may increase the half-life of the conjugate by 5%, 10%, 15%, 20%, 30%, 40%, 50%, 60%, 70%, 80%, 90%. 100%, 200%, 300%, 400%, 500% or more relative to a conjugate having the corresponding Fc domain without the mutation that enhances FcRn binding. Exemplary Fc domains with enhanced binding to the FcRN and methods for making Fc domains having enhanced binding to the FcRN are known in the art, for example, as described in Maeda, A. et al., Identification of human IgG1 variant with enhanced FcRn binding and without increased binding to rheumatoid factor autoantibody, MABS, 2017, 9(5):844-853, which is incorporated herein in its entirety.

As used herein, an amino acid “corresponding to” a particular amino acid residue (e.g., of a particular SEQ ID NO:) should be understood to include any amino acid residue that one of skill in the art would understand to align to the particular residue (e.g., of the particular sequence). For example, any one of the sequences described in WO 2020/051498, WO 2020/252393, WO 2020/252396, WO 2021/046549, or WO 2021/050612 may be mutated to include a YTE mutation, an LS mutation, and/or an N434H mutation by mutating the “corresponding residues” of the amino acid sequence.

As used herein, a sulfur atom “corresponding to” a particular cysteine residue of a particular SEQ ID NO. should be understood to include the sulfur atom of any cysteine residue that one of skill in the art would understand to align to the particular cysteine of the particular sequence. The protein sequence alignment of human IgG1 (UniProtKB: P01857), human IgG2 (UniProtKB: P01859), human IgG3 (UniProtKB: P01860), and human IgG4 (UniProtKB: P01861) is provided in WO 2020/051498, WO 2020/252393, WO 2020/252396, WO 2021/046549, or WO 2021/050612. One of skill in the art would readily be able to perform such an alignment with any IgG variant of the invention to determine the sulfur atom of a cysteine that corresponds to any sulfur atom of a particular cysteine of a particular SEQ ID NO: described in WO 2020/051498, WO 2020/252393, WO 2020/252396, WO 2021/046549, or WO 2021/050612.

As used herein, a nitrogen atom “corresponding to” a particular lysine residue of a particular SEQ ID NO. should be understood to include the nitrogen atom of any lysine residue that one of skill in the art would understand to align to the particular lysine of the particular sequence. The protein sequence alignment of human IgG1 (UniProtKB: P01857), human IgG2 (UniProtKB: P01859), human IgG3 (UniProtKB: P01860), and human IgG4 (UniProtKB: P01861) is provided in WO 2020/051498, WO 2020/252393, WO 2020/252396, WO 2021/046549, or WO 2021/050612. One of skill in the art would readily be able to perform such an alignment with any IgG variant of the invention to determine the nitrogen atom of a lysine that corresponds to any nitrogen atom of a particular lysine of a particular SEQ ID NO: described in WO 2020/051498, WO 2020/252393, WO 2020/252396, WO 2021/046549, or WO 2021/050612.

In some embodiments, the Fc domain monomer includes less than about 300 amino acid residues (e.g., less than about 300, less than about 295, less than about 290, less than about 285, less than about 280, less than about 275, less than about 270, less than about 265, less than about 260, less than about 255, less than about 250, less than about 245, less than about 240, less than about 235, less than about 230, less than about 225, or less than about 220 amino acid residues). In some embodiments, the Fc domain monomer is less than about 40 kDa (e.g., less than about 35 kDa, less than about 30 kDa, less than about 25 kDa).

In some embodiments, the Fc domain monomer includes at least 200 amino acid residues (e.g., at least 210, at least 220, at least 230, at least 240, at least 250, at least 260, at least 270, at least 280, at least 290, or at least 300 amino residues). In some embodiments, the Fc domain monomer is at least 20 kDa (e.g., at least 25 kDa, at least 30 kDa, or at least 35 kDa).

In some embodiments, the Fc domain monomer includes 200 to 400 amino acid residues (e.g., 200 to 250, 250 to 300, 300 to 350, 350 to 400, 200 to 300, 250 to 350, or 300 to 400 amino acid residues). In some embodiments, the Fc domain monomer is 20 to 40 kDa (e.g., 20 to 25 kDa, 25 to 30 kDa, 35 to 40 kDa, 20 to 30 kDa, 25 to 35 kDa, or 30 to 40 KDa).

In some embodiments, the Fc domain monomer includes an amino acid sequence at least 90% identical (e.g., at least 95%, at least 98%) to the sequence described in WO 2020/051498, WO 2020/252393, WO 2020/252396, WO 2021/046549, or WO 2021/050612 or a region thereof. In some embodiments, the Fc domain monomer includes the amino acid sequence described in WO 2020/051498, WO 2020/252393, WO 2020/252396, WO 2021/046549, or WO 2021/050612 or a region thereof.

In some embodiments, the region includes at least 40 amino acid residues, at least 50 amino acid residues, at least 60 amino acid residues, at least 70 amino acids residues, at least 80 amino acids residues, at least 90 amino acid residues, at least 100 amino acid residues, at least 110 amino acid residues, at least 120 amino residues, at least 130 amino acid residues, at least 140 amino acid residues, at least 150 amino acid residues, at least 160 amino acid residues, at least 170 amino acid residues, at least 180 amino acid residues, at least 190 amino acid residues, or at least 200 amino acid residues.

### III. Proteins of the Protein-Drug Conjugates: Albumin Proteins or Albumin Protein-Binding Peptides

The protein-drug conjugates disclosed herein include a polypeptide, E (e.g., Fc domain monomer, an Fc domain, an Fc-binding peptide, an albumin protein, or an albumin protein-binding peptide) conjugated to one or more therapeutic agents through one or more linkers.

### Albumin Proteins

An albumin protein of the invention may be a naturally-occurring albumin or a variant thereof, such as an engineered variant of a naturally-occurring albumin protein. Variants include polymorphisms, fragments such as domains and sub-domains, and fusion proteins. An albumin protein may include the sequence of an albumin protein obtained from any source. Preferably the source is mammalian, such as human or bovine. Most preferably, the albumin protein is human serum albumin (HSA), or a variant thereof. Human serum albumins include any albumin protein having an amino acid sequence naturally occurring in humans, and variants thereof. An albumin protein coding sequence is obtainable by methods know to those of skill in the art for isolating and sequencing cDNA corresponding to human genes. An albumin protein of the invention may include the amino acid sequence of human serum albumin (HSA), provided in WO 2020/051498, WO 2020/252393, WO 2020/252396, WO 2021/046549, or WO 2021/050612 or the amino acid sequence of mouse serum albumin (MSA), provided in WO 2020/051498, WO 2020/252393, WO 2020/252396, WO 2021/046549, or WO 2021/050612 or a variant or fragment thereof, preferably a functional variant or fragment thereof. A fragment or variant may or may not be functional, or may retain the function of albumin to some degree. For example, a fragment or variant may retain the ability to bind to an albumin receptor, such as HSA or MSA, by at least 10%, 20%, 30%, 40%, 50%, 60%, 70%, 80%, 90%, 100%, or 105% of the ability of the parent albumin (e.g., the parent albumin from which the fragment or variant is derived). Relative binding ability may be determined by methods known in the art, such as by surface plasmon resonance.

The albumin protein may be a naturally-occurring polymorphic variant of an albumin protein, such as human serum albumin. Generally, variants or fragments of human serum albumin will have at least 5%, 10%, 15%, 20%, 30%, 40%, 50%, 60%, or 70%, and preferably 80%, 90%, 95%, 100%, or 105% or more of human serum albumin or mouse serum albumin's ligand binding activity.

The albumin protein may include the amino acid sequence of bovine serum albumin. Bovine serum albumin proteins include any albumin having an amino acid sequence naturally occurring in cows, for example, as described by Swissprot accession number P02769, and variants thereof as defined herein. Bovine serum albumin proteins also includes fragments of full-length bovine serum albumin or variants thereof, as defined herein.

The albumin protein may comprise the sequence of an albumin derived from one of serum albumin from dog (e.g., Swissprot accession number P49822-1), pig (e.g., Swissprot accession number P08835-1), goat (e.g., Sigma product no. A2514 or A4164), cat (e.g., Swissprot accession number P49064-1), chicken (e.g., Swissprot accession number P19121-1), ovalbumin (e.g., chicken ovalbumin) (e.g., Swissprot accession number P01012-1), turkey ovalbumin (e.g., Swissprot accession number 073860-1), donkey (e.g., Swissprot accession number Q5XLE4-1), guinea pig (e.g., Swissprot accession number Q6WDN9-1), hamster (e.g., as described in DeMarco et al. International Journal for Parasitology 37(11): 1201-1208 (2007)), horse (e.g., Swissprot accession number P35747-1), rhesus monkey (e.g., Swissprot accession number Q28522-1), mouse (e.g., Swissprot accession number P07724-1), pigeon (e.g., as defined by Khan et al. Int. J. Biol. Macromol. 30(3-4), 171-8 (2002)), rabbit (e.g., Swissprot accession number P49065-1), rat (e.g., Swissprot accession number P02770-1) or sheep (e.g., Swissprot accession number P14639-1), and includes variants and fragments thereof as defined herein.

Many naturally-occurring mutant forms of albumin are known to those skilled in the art. Naturally-occurring mutant forms of albumin are described in, for example, Peters, et al. All About Albumin: Biochemistry, Genetics and Medical Applications, Academic Press, Inc., San Diego, Calif., p.170-181 (1996).

Albumin proteins of the invention include variants of naturally-occurring albumin proteins. A variant albumin refers to an albumin protein having at least one amino acid mutation, such as an amino acid mutation generated by an insertion, deletion, or substitution, either conservative or non-conservative, provided that such changes result in an albumin protein for which at least one basic property has not been significantly altered (e.g., has not been altered by more than 5%, 10%, 15%, 20%, 25%, 30%, 35%, or 40%). Exemplary properties which may define the activity of an albumin protein include binding activity (e.g., including binding specificity or affinity to bilirubin, or a fatty acid such as a long-chain fatty acid), osmolarity, or behavior in a certain pH-range.

Typically an albumin protein variant will have at least 40%, at least 50%, at least 60%, and preferably at least 70%, at least 80%, at least 90%, at least 95%, at least 96%, at least 97%, at least 98%, or at least 99% amino acid sequence identity with a naturally-occurring albumin protein, such as the albumin protein described in WO 2020/051498, WO 2020/252393, WO 2020/252396, WO 2021/046549, or WO 2021/050612.

Methods for the production and purification of recombinant human albumins are well-established (Sleep et al. Biotechnology, 8(1):42-6 (1990)), and include the production of recombinant human albumin for pharmaceutical applications (Bosse et al. J Clin Pharmacol 45(1):57-67 (2005)). The three-dimensional structure of HSA has been elucidated by X-ray crystallography (Carter et al. Science. 244(4909): 1195-8(1998)); Sugio et al. Protein Eng. 12(6):439-46 (1999)). The HSA polypeptide chain has 35 cysteine residues, which form 17 disulfide bonds, and one unpaired (e.g., free) cysteine at position 34 of the mature protein. Cys-34 of HSA has been used for conjugation of molecules to albumin (Leger et al. Bioorg Med Chem Lett 14(17):4395-8 (2004); Thibaudeau et al. Bioconjug Chem 16(4):1000-8 (2005)), and provides a site for site-specific conjugation.

### Conjugation of Albumin Proteins

An albumin protein of the invention may be conjugated to (e.g., by way of a covalent bond) to any therapeutic agent. The albumin protein may be conjugated to any compound of the invention by any method well-known to those of skill in the art for producing small-molecule-protein conjugates. This may include covalent conjugation to a solvent-exposed amino acid, such as a solvent exposed cysteine or lysine.

An albumin protein of the invention may be conjugated to any compound of the invention by way of an amino acid located within 10 amino acid residues of the C-terminal or N-terminal end of the albumin protein. An albumin protein may include a C-terminal or N-terminal polypeptide fusion of 1, 2, 3, 4, 5, 6, 7, 8, 9, 10, 15, or 20 or more amino acid. The C-terminal or N-terminal polypeptide fusion may include one or more solvent-exposed cysteine or lysine residues, which may be used for covalent conjugation of a therapeutic agent, A¹.

Albumin proteins of the invention include any albumin protein which has been engineered to include one or more solvent-exposed cysteine or lysine residues, which may provide a site for conjugation to a compound of the invention (e.g., conjugation to therapeutic agent, A¹, including by way of a linker). Most preferably, the albumin protein will contain a single solvent-exposed cysteine or lysine, thus enabling site-specific conjugation of a compound of the invention.

Exemplary methods for the production of engineered variants of albumin proteins that include one or more conjugation-competent cysteine residues are provided in U.S. Patent Application No. 2017/0081389, which is incorporated herein by reference in its entirety. Briefly, preferred albumin protein variants are those comprising a single, solvent-exposed, unpaired (e.g., free) cysteine residue, thus enabling site-specific conjugation of a linker to the cysteine residue.

Albumin proteins which have been engineered to enable chemical conjugation to a solvent-exposed, unpaired cysteine residue include the albumin protein described in WO 2020/051498, WO 2020/252393, WO 2020/252396, WO 2021/046549, or WO 2021/050612.

In some embodiments of the invention, the net result of the substitution, deletion, addition, or insertion events of (a), (b), (c) and/or (d) is that the number of conjugation competent cysteine residues of the polypeptide sequence is increased relative to the parent albumin sequence. In some embodiments of the invention, the net result of the substitution, deletion, addition, or insertion events of (a), (b), (c) and/or (d) is that the number of conjugation competent-cysteine residues of the polypeptide sequence is one, thus enabling site-specific conjugation.

Preferred albumin protein variants also include albumin proteins having a single solvent-exposed lysine residue, thus enabling site-specific conjugation of a linker to the lysine residue. Such variants may be generated by engineering an albumin protein, including any of the methods previously described (e.g., insertion, deletion, substitution, or C-terminal or N-terminal fusion).

### Albumin Protein-Binding Peptides

Conjugation of a biologically-active compound to an albumin protein-binding peptide can alter the pharmacodynamics of the biologically-active compound, including the alteration of tissue uptake, penetration, and diffusion. In a preferred embodiment, conjugation of an albumin protein-binding peptide to a therapeutic agent, A¹, increases the efficacy or decreases the toxicity of the compound, as compared to the compound alone.

Albumin protein-binding peptides of the invention include any polypeptide having an amino acid sequence of 5 to 50 (e.g., 5 to 40, 5 to 30, 5 to 20, 5 to 15, 5 to 10, 10 to 50, 10 to 30, or 10 to 20) amino acid residues that has affinity for and functions to bind an albumin protein, such as any of the albumin proteins described herein. Preferably, the albumin protein-binding peptide binds to a naturally occurring serum albumin, most preferably human serum albumin. An albumin protein-binding peptide can be of different origins, e.g., synthetic, human, mouse, or rat. Albumin protein-binding peptides of the invention include albumin protein-binding peptides which have been engineered to include one or more (e.g., two, three, four, or five) solvent-exposed cysteine or lysine residues, which may provide a site for conjugation to a therapeutic agent, A¹, including by way of a linker). Most preferably, the albumin protein-binding peptide will contain a single solvent-exposed cysteine or lysine, thus enabling site-specific conjugation of a compound of the invention. Albumin protein-binding peptides may include only naturally occurring amino acid residues, or may include one or more non-naturally occurring amino acid residues. Where included, a non-naturally occurring amino acid residue (e.g., the side chain of a non-naturally occurring amino acid residue) may be used as the point of attachment for a therapeutic agent, A¹. Albumin protein-binding peptides of the invention may be linear or cyclic. Albumin protein-binding peptides of the invention include any albumin protein-binding peptides known to one of skill in the art, examples of which, are provided in WO 2020/051498, WO 2020/252393, WO 2020/252396, WO 2021/046549, or WO 2021/050612.

Albumin protein-binding peptide, and conjugates including an albumin protein-binding peptide, preferably bind an albumin protein (e.g., human serum albumin) with an affinity characterized by a dissociation constant, Kd, that is less than about 100 μM, preferably less than about 100 nM, and most preferably do not substantially bind other plasma proteins. Specific examples of such compounds are linear or cyclic peptides, preferably between about 10 and 20 amino acid residues in length, optionally modified at the N-terminus or C-terminus or both.

Albumin protein-binding peptides include linear and cyclic peptides described in WO 2020/051498, WO 2020/252393, WO 2020/252396, WO 2021/046549, or WO 2021/050612.

Further exemplary albumin protein-binding peptides are provided in U.S. Patent Application No. 2005/0287153, which is incorporated herein by reference in its entirety.

### Conjugation of Albumin Protein-Binding Peptides

An albumin protein-binding peptide of the invention may be conjugated to (e.g., by way of a covalent bond) to any therapeutic agent, A¹. The albumin protein-binding peptide may be conjugated to any compound of the invention by any method known to those of skill in the art for producing peptide-small molecule conjugates. This may include covalent conjugation to the side chain group of an amino acid residue, such as a cysteine, a lysine, or a non-natural amino acid. Alternately, covalent conjugation may occur at the C-terminus (e.g., to the C-terminal carboxylic acid, or to the side chain group of the C-terminal residue) or at the N-terminus (e.g., to the N-terminal amino group, or to the side chain group of the N-terminal amino acid).

### IV. Linkers of Protein-Drug Conjugates

A linker refers to a linkage or connection between two or more components in a protein-drug conjugate described herein (e.g., between W and A¹, between W and G, between G and A¹, between W and E, and/or between E and A¹).

### Conjugation Chemistries

In the methods disclosed herein, compounds of formula (M-I) or (M-II) are conjugated to a polypeptide, E (e.g., Fc domain monomer, an Fc domain, an Fc-binding peptide, an albumin protein, or an albumin protein-binding peptide (e.g., by way of a linker)), using intermediate compounds of formula (F-I) or (F-II), which are functionalized with a phenyl ester group (e.g., a trifluorophenyl ester group or a tetrafluorophenyl ester group). Conjugation (e.g., by acylation) of E and the intermediate compound of formula (F-I) or (F-II) forms a conjugate, for example a conjugate described by any one of formulas (M-I) and (M-II).

Intermediate compounds of formula (F-I) or (F-II) can be synthesized by reacting a phenol (e.g., tetrafluorophenol or trifluorophenol) with a compound comprising a therapeutic agent, A¹, and a linker including an activated carboxylic acid.

Intermediate compounds of formula (F-I) or (F-II) can also be synthesized by reacting a compound comprising a functional group (e.g., G^{a}), a linker (e.g., L²), and a phenyl ester (e.g., trifluorophenyl ester or tetrafluorophenyl ester) with a compound comprising a functional group (e.g., G^{b}), a linker (e.g., L³), and a therapeutic agent, A¹.

Reaction of two or more components in an intermediate compound (e.g., a compound of formula (F-I) or (F-II)) may be accomplished using well-known organic chemical synthesis techniques and methods. Complementary functional groups (e.g., G^{a} and G^{b}) on two components may react with each other to form a covalent bond. Complementary functional groups (e.g., G^{a} and G^{b}) on two components may react with each other to form a chemical moiety, e.g., G. Examples of complementary reactive functional groups include, but are not limited to, e.g., maleimide and cysteine, amine and activated carboxylic acid (e.g., to form an amide linkage), thiol and maleimide, activated sulfonic acid and amine, isocyanate and amine, azide and alkyne (e.g., click chemistry to form a triazole), and alkene and tetrazine.

Other examples of functional groups capable of reacting with amino groups include, e.g., alkylating and acylating agents. Representative alkylating agents include: (i) an α-haloacetyl group, e.g., XCH₂CO— (where X=Br, Cl, or I); (ii) a N-maleimide group, which may react with amino groups either through a Michael type reaction or through acylation by addition to the ring carbonyl group; (iii) an aryl halide, e.g., a nitrohaloaromatic group; (iv) an alkyl halide; (v) an aldehyde or ketone capable of Schiff's base formation with amino groups; (vi) an epoxide, e.g., an epichlorohydrin and a bisoxirane, which may react with amino, sulfhydryl, or phenolic hydroxyl groups; (vii) a chlorine-containing of s-triazine, which is reactive towards nucleophiles such as amino, sulfhydryl, and hydroxyl groups; (viii) an aziridine, which is reactive towards nucleophiles such as amino groups by ring opening; (ix) a squaric acid diethyl ester; and (x) an α-haloalkyl ether.

Examples of amino-reactive acylating groups include, e.g., (i) an isocyanate and an isothiocyanate; (ii) a sulfonyl chloride; (iii) an acid halide; (iv) an active ester, e.g., a nitrophenylester or N-hydroxysuccinimidyl ester; (v) an acid anhydride, e.g., a mixed, symmetrical, or N-carboxyanhydride; (vi) an acylazide; and (vii) an imidoester. Aldehydes and ketones may be reacted with amines to form Schiffs bases, which may be stabilized through reductive amination.

It will be appreciated that certain functional groups may be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

In some embodiments, the intermediate compound (e.g., a compound of formula (F-I) or (F-II)) is synthesized via click chemistry (e.g., where G^{a} of formula (G3-A) is an azido group and G^{b} of formula (G3-B) is an alkynyl group; or where G^{a} of formula (G3-A) is an alkynl group and G^{b} of formula (G3-B) is an azido group). In some embodiments, the click chemistry includes the use of a Cu(I) source.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. General Procedure for Synthesis Using Phenyl Esters

A phenyl ester group (e.g., a trifluorophenyl ester group or tetrafluorophenyl ester group) may be used to form an amide linkage between two components. For example, a first component (e.g., a compound) attached to a phenyl ester group may be reacted with a second component (e.g., a protein or polymer) attached to a group including an amino group to form a structure (e.g., conjugate) including an amide linkage (e.g., —C(O)NH— or —NHC(O)—).

A scheme illustrating this transformation between a first component (e.g., Y¹) attached to a tetrafluorophenyl ester or trifluorophenyl ester group and a second component (e.g., Y²) attached to a 1-aminoalkyl group is shown below.

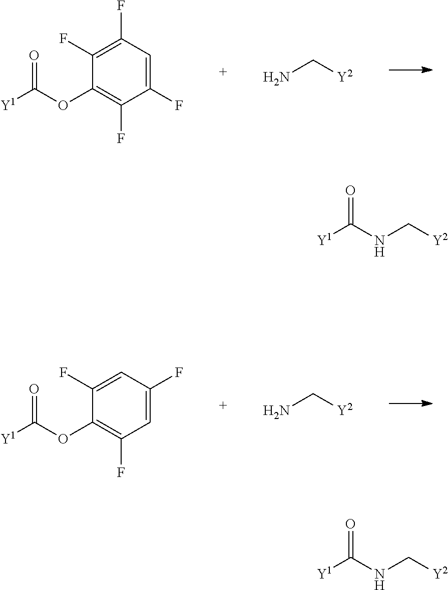

The first component (Y¹) may be a compound that includes a linker. The second component (Y²) may be a protein including, e.g., a lysine residue, or a polymer substituted with an amino group, e.g., a primary amino group.

### Example 2. General Procedure for Synthesis of Conjugates Using Tetrafluorophenyl Ester Intermediates and Trifluorophenyl Ester Intermediates

### Tetrafluorophenyl Ester

A solution of Fc in pH 7.4 PBS buffer was treated with a solution of tetrafluorophenyl ester intermediate dissolved in DMF. The pH was adjusted to ˜7.5 to 8.0 with borate buffer (pH ˜8.5). The solution was then gently rocked at room temperature. The crude conjugate was purified by dialysis in arginine buffer (200 mM Arginine, 120 mM NaCl, 1% Sucrose pH 6.0). DAR is determined by Maldi-TOF of the purified conjugates.

### Trifluoro Phenyl Ester Intermediates

A solution of Fc in pH 7.4 PBS buffer was treated with a solution of trifluorophenyl ester intermediate dissolved in DMF. The pH was adjusted to ˜8.5 to 9.5 with borate buffer (pH ˜8.5 to 9.5). the solution was then gently rocked at room temperature. The crude conjugate was purified by dialysis in arginine buffer (200 mM Arginine, 120 mM NaCl, 1% Sucrose pH 6.0). DAR is determined by Maldi-TOF of the purified conjugates.

### Example 3. Synthesis of Conjugates Using Tetrafluorophenyl Ester Intermediates

The following conjugates were prepared following the general procedure described in Example 1.

### Conjugate 3A

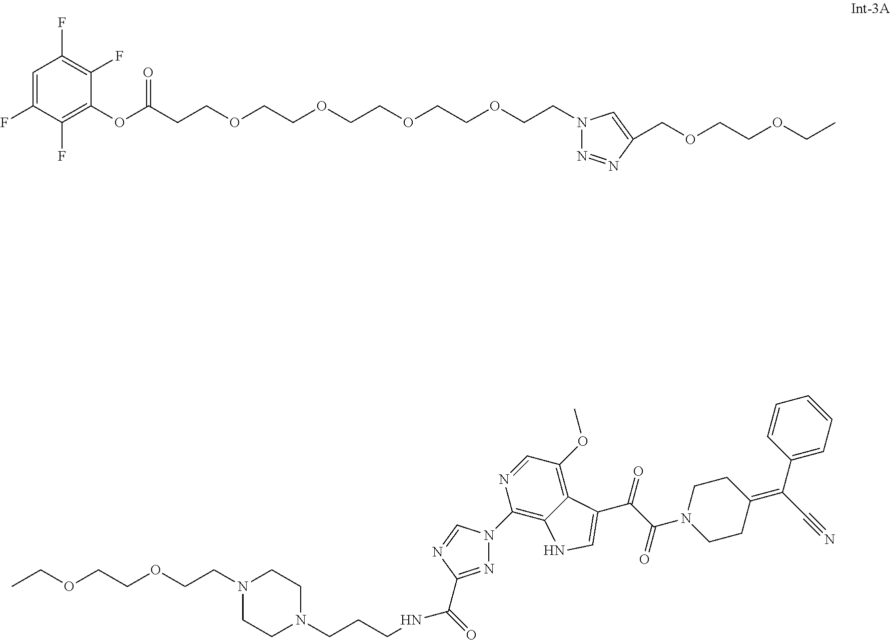

A solution of polypeptide having sequence of SEQ ID NO: 2 in PBS buffer (pH=7.4) and DMF was treated with a solution of tetrafluorophenyl ester (Int-3A) dissolved in DMF. The pH was adjusted to ˜7.5 to 8.0 with borate buffer (pH 8.5). The mixture was then gently rocked at room temperature. Maldi TOF after 3 hours shows an average DAR (drug-to-antibody ratio) of 3 to 5. The crude conjugate was purified by dialysis in arginine buffer (200 mM Arginine, 120 mM NaCl, 1% Sucrose pH 6.0). DAR is determined by Maldi TOF of the purified conjugates. Yield=67%. Maldi-TOF=61,737.

DAR (average)=3.2.

### Conjugate 3B

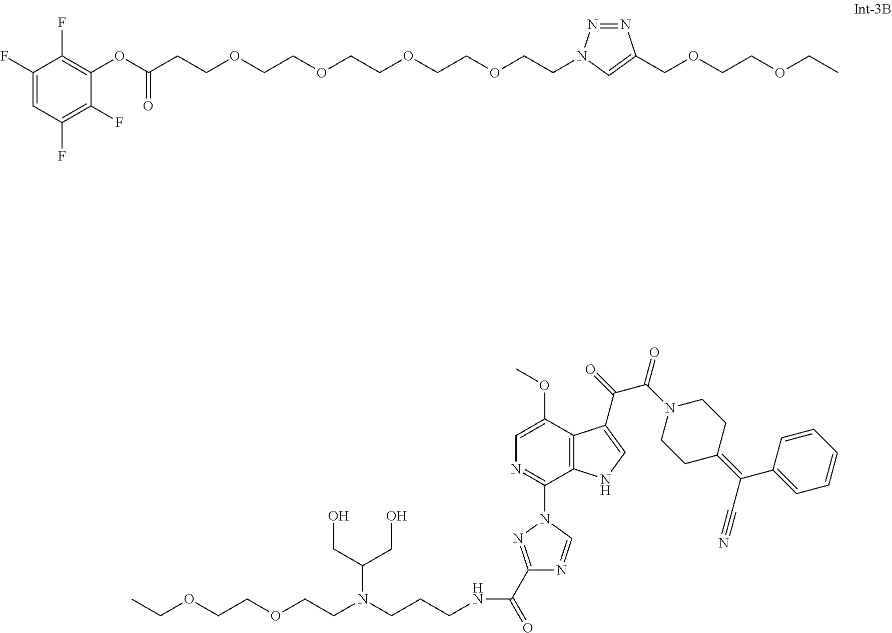

A solution of polypeptide having sequence of SEQ ID NO: 2 in PBS buffer (pH=7.4) and DMF was treated with a solution of tetrafluorophenyl ester (Int-3B) dissolved in DMF. The pH was adjusted to ˜7.5 to 8.0 with borate buffer (pH 8.5). The mixture was then gently rocked at room temperature. Maldi-TOF after 3 hours shows an average DAR (drug-to-antibody ratio) of 3 to 5. The crude conjugate was purified by dialysis in arginine buffer (200 mM Arginine, 120 mM NaCl, 1% Sucrose pH 6.0). DAR is determined by Maldi-TOF of the purified conjugates. Yield=66%. Maldi-TOF=59,674.

DAR (average)=1.3.

### Example 4. Synthesis of Conjugates Using Trifluorophenyl Ester Intermediates

Trifluorophenyl ester compounds (e.g., compounds of formula (F-I), (F-II), (F-II-A), (F-II-B), (G1-A), and (G2-A)) can provide further advantages in the synthesis of protein-drug conjugates. For example, trifluorophenyl ester compounds can exhibit increased stability, which allows for, e.g., purification by reverse phase chromatography and lyophilization with minimal hydrolysis of the activated ester.

The following conjugates were prepared following the general procedure described in Example 2.

### Conjugate 4A

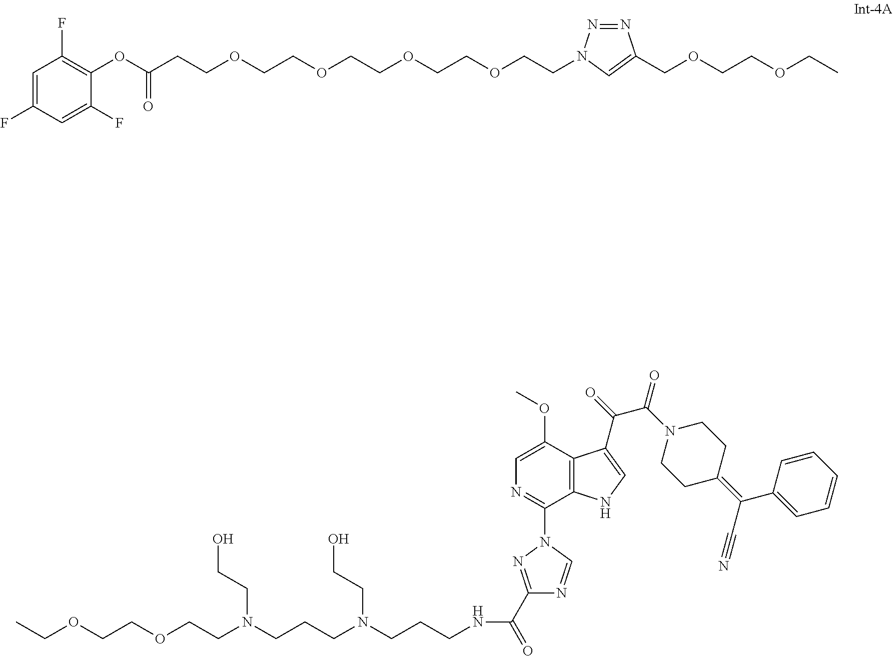

A solution of polypeptide having a sequence of SEQ ID NO: 2 in PBS buffer (pH=7.4) and DMF was treated with a solution of trifluorophenyl ester (Int-4A) dissolved in DMF. The pH was adjusted to ˜8.0 to 9.5 with borate buffer (pH 8.5-9.5). Then the mixture was gently rocked at room temperature. Maldi TOF after 3 hours shows an average DAR of 3 to 5. The crude conjugate was purified by dialysis in arginine buffer (200 mM Arginine, 120 mM NaCl, 1% Sucrose pH 6.0). DAR is determined by Maldi-TOF of the purified. Yield=87%. Maldi-TOF=59,779. DAR (average)=1.3.

### Conjugate 4B

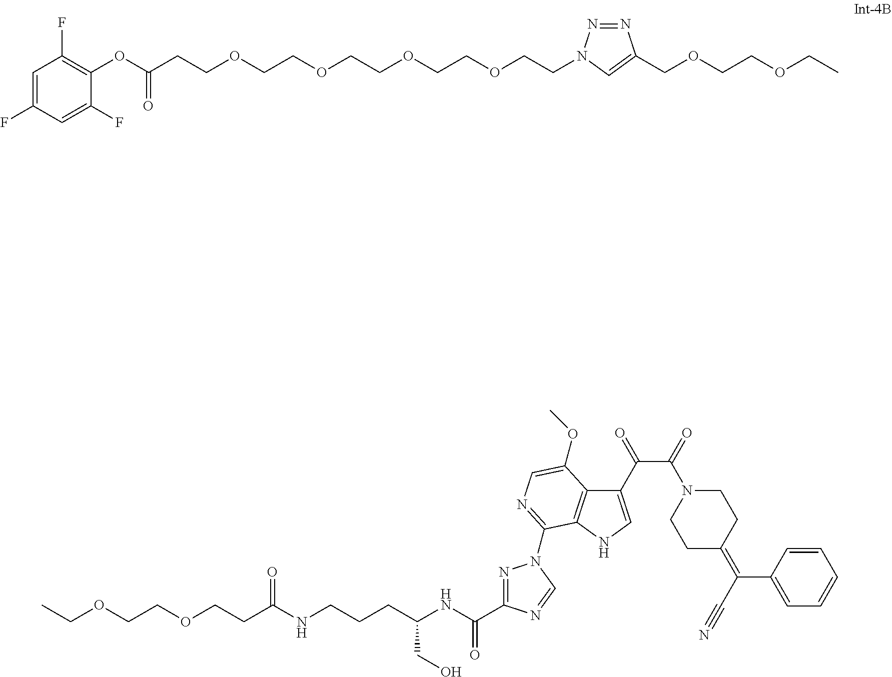

A solution of polypeptide having a sequence of SEQ ID NO: 2 in PBS buffer (pH=7.4) and DMF was treated with a solution of trifluorophenyl ester (Int-4B) dissolved in DMF. The pH was adjusted to ˜8.0 to 9.5 with borate buffer (pH 8.5-9.5). Then the mixture was gently rocked at room temperature. Maldi TOF after 3 hours shows an average DAR of 3 to 5. The crude conjugate was purified by dialysis in arginine buffer (200 mM Arginine, 120 mM NaCl, 1% Sucrose pH 6.0). DAR is determined by Maldi TOF of the purified. Yield=80%. Maldi-TOF=61,821. DAR (average)=3.2.

### Example 5. Synthesis of Conjugates Using Tetrafluorophenyl Ester Intermediates

The following conjugates were prepared following the general procedure described in Example 1.

### Conjugate 5A

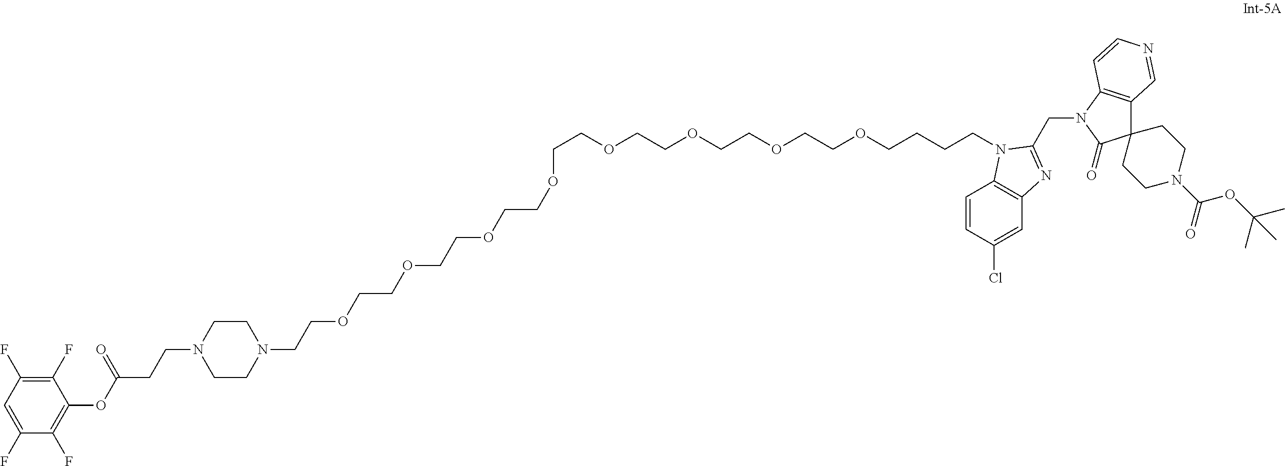 [SMILES: CC(C)(C)OC(=O)N1CCC2(CC1)C(=O)N(Cc1nc3cc(Cl)ccc3n1CCCCOCCOCCOCCOCCOCCOCCOCCOCCN1CCN(CCC(=O)Oc3c(F)c(F)cc(F)c3F)CC1)c1ccncc12]

A solution of polypeptide having sequence of SEQ ID NO: 2 (0.100 g in 5.2 mL, 1.717 μmol, MW 58,218) in pH 7.4 PBS buffer was treated with a solution of tetrafluorophenyl ester (Int-5A) (0.0273 g, 17.17 μmol) dissolved in DMF (1 mL). The pH was adjusted to ˜7.5 to 8.0 with borate buffer (120 μL, 1M, pH 8.5) then was gently rocked at room temperature. Maldi-TOF after 3.0 h shows an average DAR of 3 to 5. The crude conjugate was purified by dialysis in arginine buffer (200 mM Arginine, 120 mM NaCl, 1% Sucrose pH 6.0). Yield=17.0 mg, 55.0%. Maldi-TOF=58,991. DAR=1.0.

### Conjugate 5B

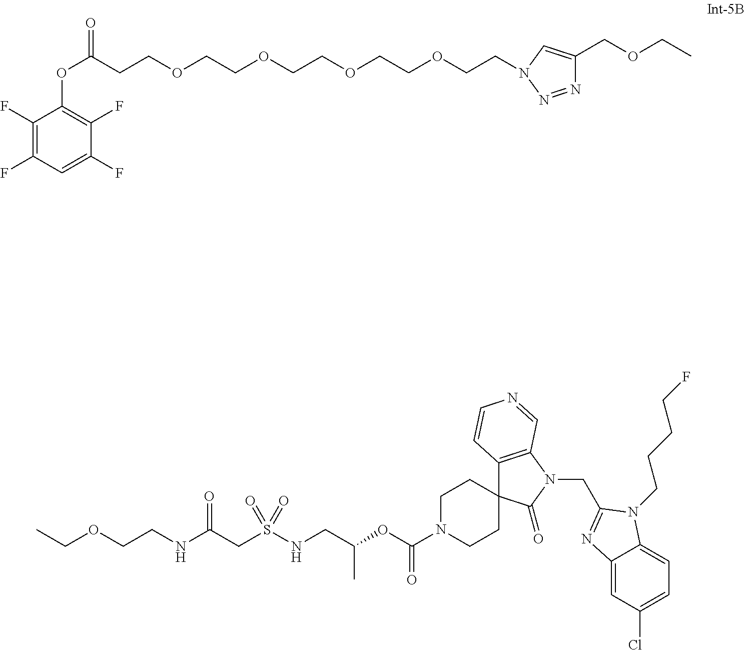

A solution of polypeptide having sequence of SEQ ID NO: 2 (0.100 g in 5.2 mL, 1.717 μmol, MW 58,218) in pH 7.4 PBS buffer was treated with a solution of tetrafluorophenyl ester (Int-5B) (0.0273 g, 17.17 μmol) dissolved in DMF (1 mL). The pH was adjusted to ˜7.5 to 8.0 with borate buffer (120 μL, 1M, pH 8.5) then was gently rocked at room temperature. Maldi TOF after 3.0 h shows an average DAR of 3 to 5. The crude conjugate was purified by dialysis in arginine buffer (200 mM Arginine, 120 mM NaCl, 1% Sucrose pH 6.0). Yield=37.9 mg, 41.0%. Maldi-TOF=62,863. DAR=4.4.

### Example 6. Synthesis of Conjugates Using Trifluorophenyl Ester Intermediates

Trifluorophenyl ester compounds (e.g., compounds of formula (F-I), (F-II), (F-II-A), (F-II-B), (G1-A), and (G2-A)) can provide further advantages in the synthesis of protein-drug conjugates. For example, trifluorophenyl ester compounds can exhibit increased stability, which allows for, e.g., purification by reverse phase chromatography and lyophilization with minimal hydrolysis of the activated ester.

The following conjugates were prepared following the general procedure described in Example 2.

### Conjugate 6A

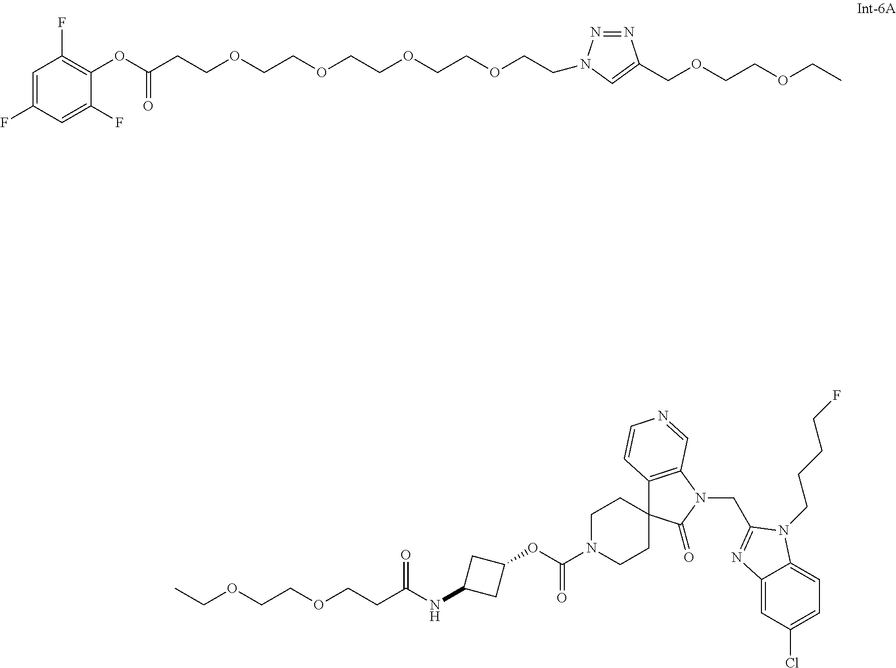

A solution of polypeptide having sequence of SEQ ID NO: 2 (0.100 g in 5.2 mL, 1.717 μmol, MW 58,218) in pH 7.4 PBS buffer was treated with a solution of trifluorophenyl ester (Int-6A) (0.0273 g, 17.17 μmol) dissolved in DMF (1 mL). The pH was adjusted to ˜8.5 with borate buffer (120 μL, 1M, pH 8.5) then was gently rocked at room temperature. Maldi-TOF after 3.0 h shows an average DAR of 3 to 5. The crude conjugate was purified by dialysis in arginine buffer (200 mM Arginine, 120 mM NaCl, 1% Sucrose pH 6.0). Yield=43.0 mg, 78.0%. Maldi-TOF=61,811. DAR=3.5.

### Conjugate 6B

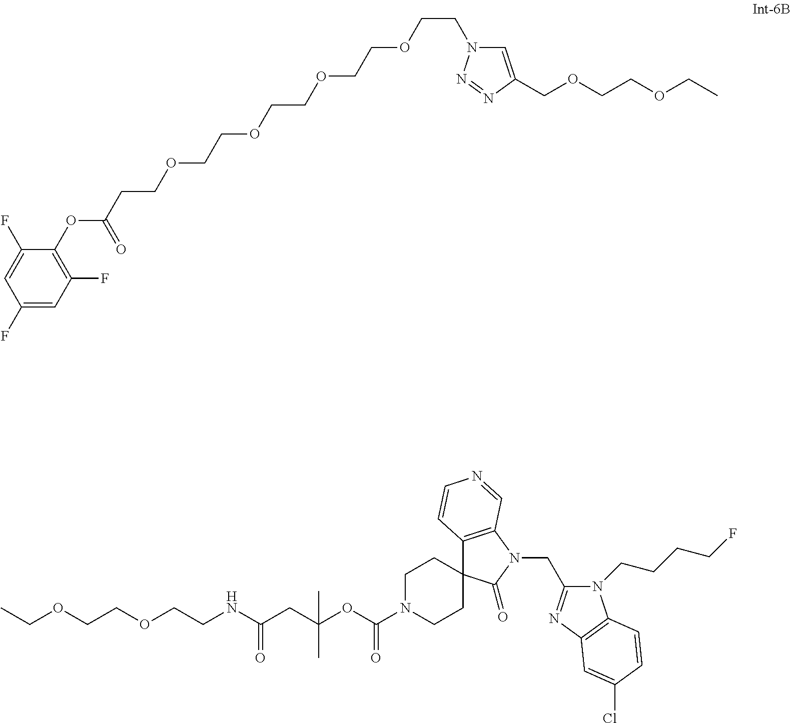

A solution of polypeptide having sequence of SEQ ID NO: 2 (0.100 g in 5.2 mL, 1.717 μmol, MW 58,218) in pH 7.4 PBS buffer was treated with a solution of trifluorophenyl ester (Int-6B) (0.0273 g, 17.17 μmol) dissolved in DMF (1 mL). The pH was adjusted to ˜8.5 with borate buffer (120 μL, 1M, pH 8.5) then was gently rocked at room temperature. Maldi-TOF after 3.0 h shows an average DAR of 3 to 5. The crude conjugate was purified by dialysis in arginine buffer (200 mM Arginine, 120 mM NaCl, 1% Sucrose pH 6.0). Yield=70.7 mg, 88.0%. Maldi-TOF=65,875. DAR=6.2.

### Conjugate 6C

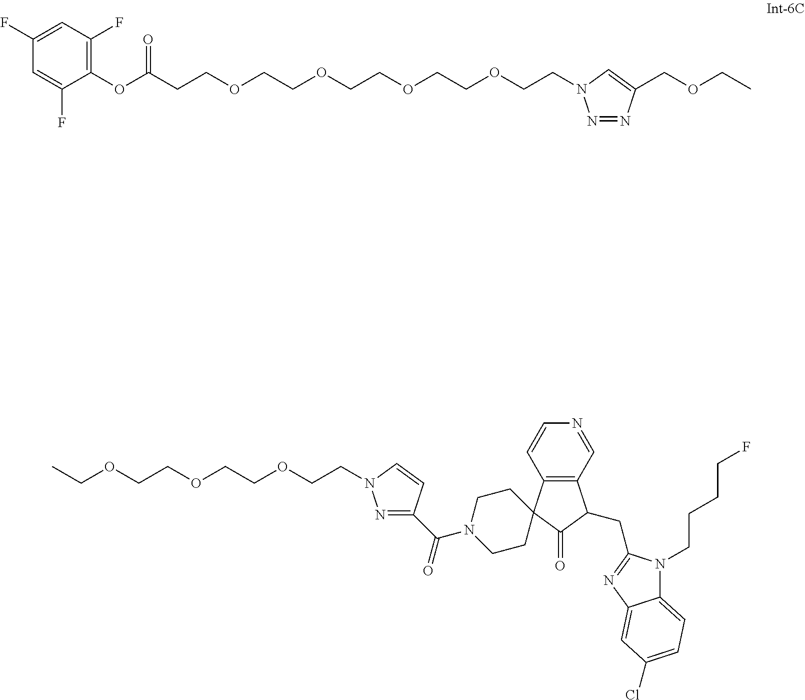

A solution of 2,4,6-trifluorophenyl active ester (Int-6C) (0.0056 g, 0.0043 mmol) in DMF (0.5 mL) was add to a polypeptide having a sequence of SEQ ID NO: 2 (0.030 g in 1.56 mL PBS at pH 7.4) then adjusted pH to ˜8.0 with borate buffer (60 μL, pH 8.5, 1.0 M). The reaction was homogeneous. Maldi TOF mass spectrometry after 4 h shows an average MW=65,345 (DAR of 7.0). The conjugation reaction was stopped by adding concentrated ammonium hydroxide (10 μL). The conjugate was purified by dialysis into 25 nM Arginine, 120 nM NaCL, and 1% sucrose pH 6.3 buffer using a Slide-d-lyzer G2 dialysis cassette (10,000 MWCO).

### Example 7. Synthesis of Int-4B

Int-4B was prepared following the procedure described below.

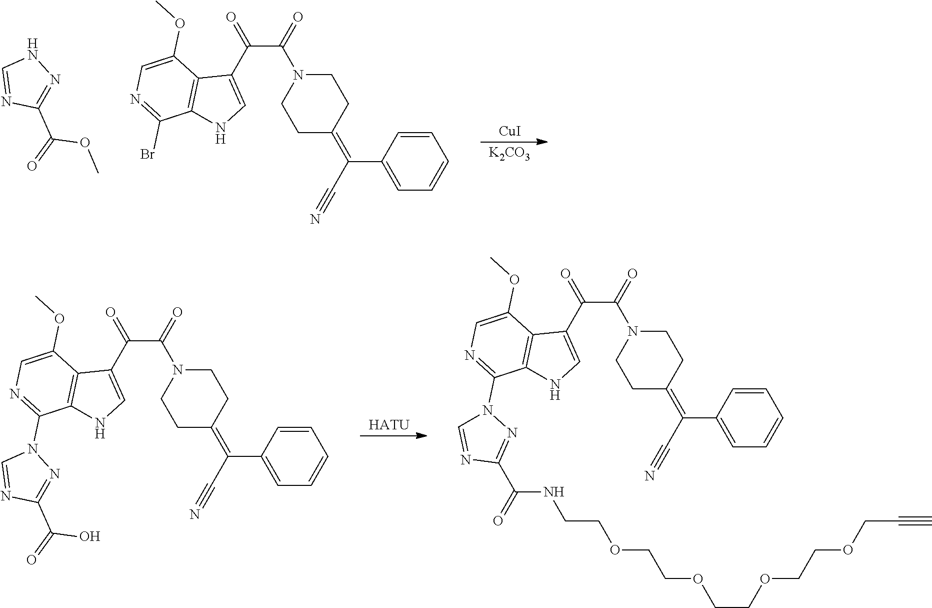

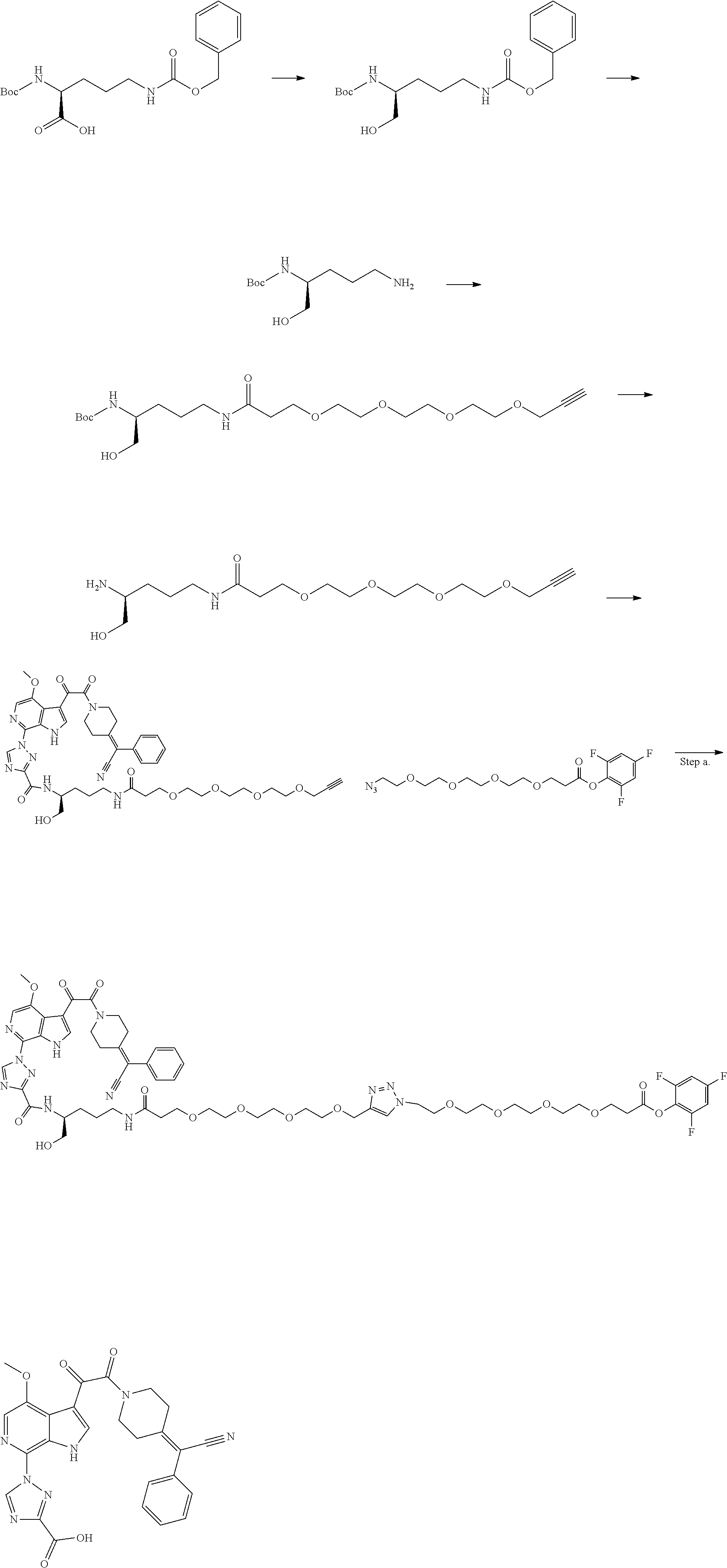

A solution of (7-bromo-4-methoxy-1H-pyrrolo[2,3-c]pyridin-3-yl)(oxo)acetic acid (2.5 g, 8.6 mmol, described in J. Med. Chem. 2018, 61(1):62-80), potassium carbonate (457 mg, 3.30 mmol), copper(I) iodide (210 mg, 1.1 mmol), 1H-1,2,4-triazol-3-carboxylate methyl ester (254 mg, 2 mmol), and (1R,2R)-N1,N2-dimethylcyclohexane-1,2-diamine (160 mg, 1.1 mmol) in 1,4-dioxane (10 mL) was heated up at 110° C. for 13 h. The reaction solution was treated with water (0.5 mL) for 15 minutes then concentrated and purified by reverse phase liquid chromatography (RPLC) using an Isco CombiFlash liquid chromatograph eluted with 20% to 80% acetonitrile and water using 0.1% TFA as the modifier. Yield of product 270 mg, 51%. Ion(s) found by LCMS: M+H=512.2.

Step b.

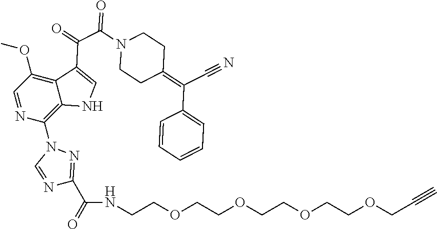 [SMILES: C#CCOCCOCCOCCOCCNC(=O)c1ncn(-c2ncc(OC)c3c(C(=O)C(=O)N4CCC(=C(C#N)c5ccccc5)CC4)c[nH]c23)n1]

To a solution of product from the previous step (1-{3-[{4-[cyano(phenyl)methylidene]piperidin-1-yl}(oxo)acetyl]-4-methoxy-1H-pyrrolo[2,3-c]pyridin-7-yl}-1H-1,2,4-triazole-3-carboxylic acid, 50 mg, 0.1 mmol) and propargyl-PEG4-amine (23 mg, 0.1 mmol) in DMF (2 ml) was added HATU (38 mg, 0.1 mmol), and N-methylmorpholine (0.07 ml, 0.5 mmol) at room temperature, and the resulting solution was stirred for 1 hour at room temperature. The solution was concentrated and purified by and purified by reverse phase liquid chromatography (RPLC) using an Isco CombiFlash liquid chromatograph eluted with 2% to 100% acetonitrile and water with 0.1% TFA as modifier. Yield of products 21 mg, 29.6%. Ion(s) found by LCMS: M+H=725.3.

Step c.

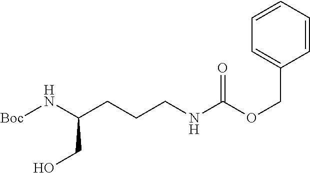 [SMILES: CC(C)(C)OC(=O)N[C@H](CO)CCCNC(=O)OCc1ccccc1]

To a −15° C. stirring solution of Nα-Boc-Nδ-Cbz-L-ornithine (1.00 g, 2.729 mmol) and N-methylmorpholine (300 uL, 2.729 mmol) in THE (10.0 mL), it was added isobutylchlorofromate (355 uL, 2.729 mmol). After stirring for 5 minutes, a freshly prepared solution of sodium borohydride (310 mg, 8.188 mmol) in water (4.0 mL) was added. Upon reaction completion, water (10 mL) was added and the temperature raised to ambient, while stirring continued for 1 h. The resulting mixture was extracted with DCM (4×30 mL), and the combined organics were dried with magnesium sulfate, filtered and concentrated per rotatory evaporation. Residual volatiles were evaporated under high vacuum. This material was used in the next step without further purification. LCMS: [(M+H]]⁺=353.2.

Step d.

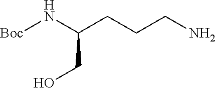 [SMILES: CC(C)(C)OC(=O)N[C@H](CO)CCCN]

Under hydrogen atmosphere, a suspension of the product from the previous step (2.729 mmol, theoretical) and 20% palladium hydroxide on carbon (500 mg) in MeOH (20 mL), was stirred until full consumption of the starting material. The mixture was filtered and the filtrate concentrated per rotatory evaporation. Residual volatiles were evaporated under high vacuum. This material was used in the next step without further purification. Ions found by LCMS: [(M+H]]⁺=219.2.

Step e.

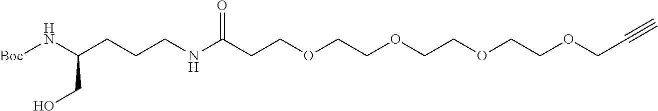 [SMILES: C#CCOCCOCCOCCOCCC(=O)NCCC[C@@H](CO)NC(=O)OC(C)(C)C]

To a 0° C. stirring solution of the product from the previous step (150 mg, 0.687 mmol), propargyl-PEG4-acid (179 mg, 0.687 mmol), and DIPEA (0.359 mL, 2.061 mmol) in DMF (4.0 mL) and DCM (0.5 mL), was added HATU (266 mg, 0.701 mmol). The temperature was raised to ambient and stirring was continued until complete as determined by LCMS. All the volatiles were removed per rotatory evaporation. The residue was purified by RP-C18 column using an Isco CombiFlash liquid chromatography eluted with 0% to 100% water and methanol, no modifier. Yield 0.186 g, 59%. Ions found by LCMS: [(M+H)]⁺=461.3.

Step f.

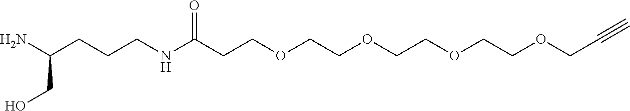 [SMILES: C#CCOCCOCCOCCOCCC(=O)NCCC[C@H](N)CO]

The product from the previous step (186 mg, 0.404 mmol) was treated with 4.0 M solution of HCl in dioxane (3.0 mL) under stirring. Upon completion, all the volatiles were evaporated per rotatory evaporation and high vacuum. This material was used in the next step without further purification. Yield 0.161 g, quant. Ions found by LCMS: [(M+H]]⁺=361.2.

Step g.

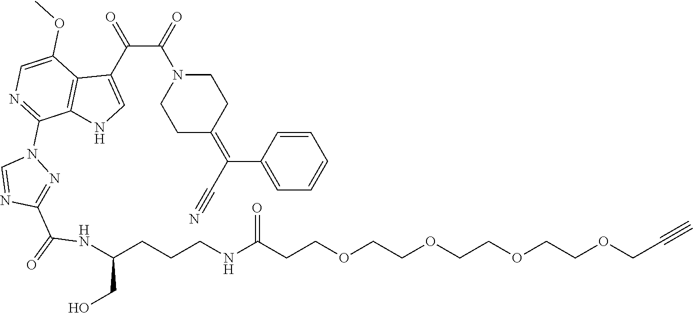 [SMILES: C#CCOCCOCCOCCOCCC(=O)NCCC[C@@H](CO)NC(=O)c1ncn(-c2ncc(OC)c3c(C(=O)C(=O)N4CCC(=C(C#N)c5ccccc5)CC4)c[nH]c23)n1]

To a 0° C. stirring solution of product from the previous step (31 mg, 0.078 mmol), intermediate i-5 (40 mg, 0.078 mmol), HOBt hydrate (36 mg, 0.235 mmol, ˜80%), and DIPEA (0.082 mL, 0.469 mmol) in DMF (3.0 mL) and DCM (0.5 mL), was added HATU (89 mg, 0.235 mmol). The temperature was raised to ambient and stirring was continued until complete as determined by LCMS. All the volatiles were removed per rotatory evaporation. The residue was purified by RP-C18 column using an Isco ACCQ liquid chromatography eluted with 0% to 100% water and acetonitrile, 0.1% TFA modifier. Yield 0.051 g, 76%. Ions found by LCMS: [(M+H)]⁺=854.2.

Step h.

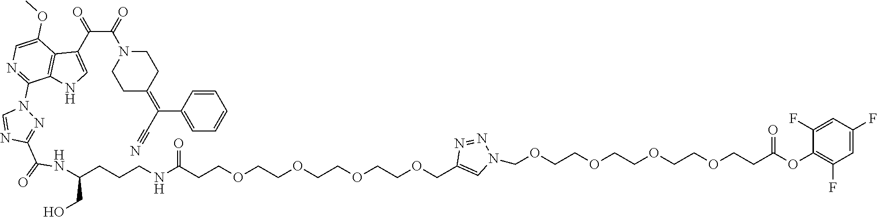 [SMILES: COc1cnc(-n2cnc(C(=O)N[C@H](CO)CCCNC(=O)CCOCCOCCOCCOCc3cn(COCCOCCOCCOCCC(=O)Oc4c(F)cc(F)cc4F)nn3)n2)c2[nH]cc(C(=O)C(=O)N3CCC(=C(C#N)c4ccccc4)CC3)c12]

To a stirring solution of the product from the previous step (0.047 mg, 0.055 mmol), azido-PEG4-trifluorophenyl ester (24 mg, 0.058 mmol), BTTAA (1.2 mg, 0.0027 mmol), cupric sulfate (0.2 mg, 0.0014 mmol), in DMF (1.0 mL) and water (1.0 mL), it was added sodium ascorbate (5.4 mg, 0.028 mmol). Upon completion, acetic acid (0.099 mL, 1.725 mmol) was added, and the reaction was concentrated per rotatory evaporation. The residue was purified by RP-C18 column using an Isco ACCQ liquid chromatography eluted with 0% to 100% water and methanol, 0.1% TFA modifier. Yield 0.047 g, 60%. Ions found by LCMS: [(M+2H)/2]⁺=638.3.

### Example 8. Synthesis of Int-5A

Int-5A was prepared following the procedure described below.

### Synthesis of (tert-butyl 2′-oxo-1′,2′-dihydrospiro[piperidine-4, 3′-pyrrolo[2,3-c]pyridine]-1-carboxylate)

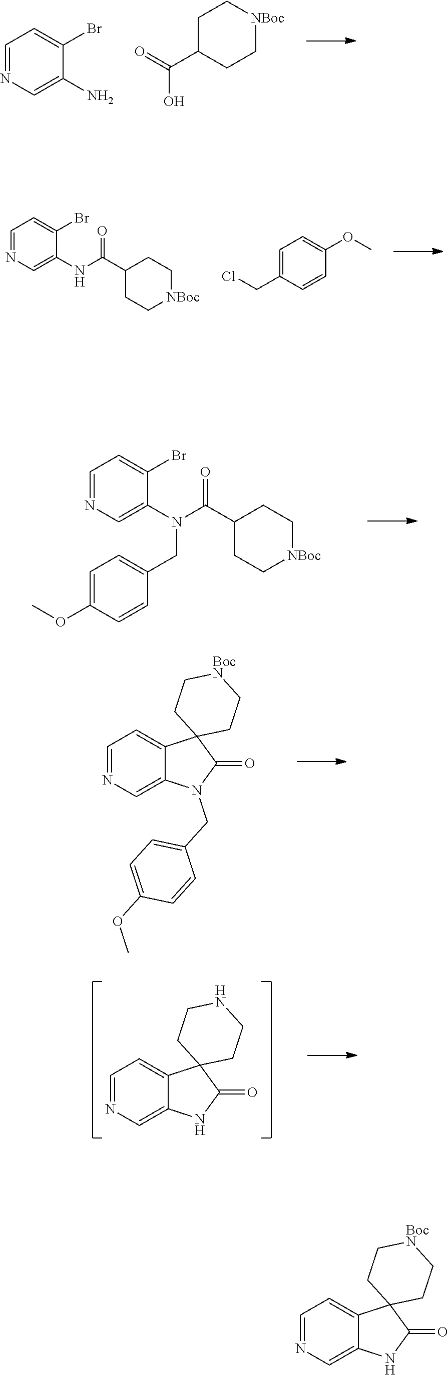

Step a.

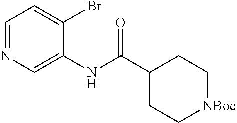 [SMILES: CC(C)(C)OC(=O)N1CCC(C(=O)Nc2cnccc2Br)CC1]

T3P (41.6 mL, 69.9 mmol, 50% by wt. in ethyl acetate) was added, dropwise over 10 minutes, to a stirring mixture of 2-amino-2-bromo-pyridine (11 g, 63.6 mmol), N-Boc-piperazine carboxylic acid (16 g, 69.9 mmol), and DIPEA (16.4 g, 127.2 mmoL) in ethyl acetated (75 mL) cooled to 0° C. The ice bath was removed and the reaction was stirred for 24 hours. The reaction mixture was diluted with water, extracted into ethyl acetate (3×, 25 mL). The combined organic extracts were dried over sodium sulfate, and concentrated on the rotary evaporator. The crude product was by silica gel chromatography on the ISCO COMBI FLASH® (15% to 100% ethyl acetate in hexanes, 25 minutes). The pure fractions were pooled and concentrated to afford the intermediate as a light orange oil. Yield 61%. LC/MS [M+H]⁺=384.2.

Step b.

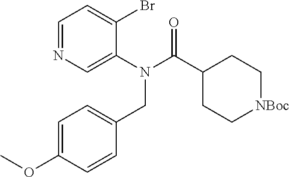 [SMILES: COc1ccc(CN(C(=O)C2CCN(C(=O)OC(C)(C)C)CC2)c2cnccc2Br)cc1]

p-Methoxy benzyl chloride (11.6 g, 74.2 mmol) was added to a mixture of the intermediate from step a. of this example (19.1 g, 49.4 mmol) and cesium carbonate (24.1 g, 74.1 mmol) in DMF (30 mL). The reaction was stirred at room temperature for 12 hours at which time it was diluted with water and extracted into ethyl acetate (3×, 30 ml). The combined organic extracts were washed with brine, dried over sodium sulfate, and concentrated on the rotary evaporator. The crude product was by silica gel chromatography on the ISCO COMBI FLASH® (10% to 100% ethyl acetate in hexanes, 25 minutes). The pure fractions were pooled and concentrated to afford the intermediate as a light orange oil. Yield 68%.

LC/MS [M+Na]⁺=526.0.

Step c.

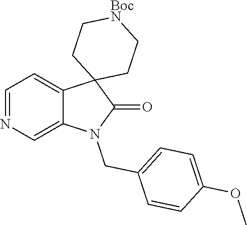 [SMILES: COc1ccc(CN2C(=O)C3(CCN(C(=O)OC(C)(C)C)CC3)c3ccncc32)cc1]

A mixture of the product from the previous step (17.0 g, 33.7 mmol), palladium(II)acetate (0.76 g, 3.4 mmol), tricyclohexyl phosphine (1.9 g, 6.7 mmol) were dissolved in dioxane (40 mL) in a sealed tube. Nitrogen was gently bubbled though the mixture for 10 minutes at which point sodium t-butoxide (4.9 g, 50.5 mmol) was added and nitrogen was bubbled through the reaction mixture for an additional 10 minutes. The tube was sealed and heated at 120° C. for 16 hours. The mixture was cooled and concentrated on the rotary evaporator. The dark, viscous product mixture was purified by silica gel chromatography on the ISCO COMBI FLASH® (0% to 10% methanol in DCM, 25 minutes). The pure fractions were pooled and concentrated to afford the intermediate as a light yellow oil. Yield 84%. LC/MS [M+H]⁺=424.2.

Step d.

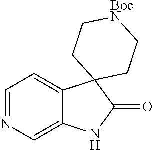 [SMILES: CC(C)(C)OC(=O)N1CCC2(CC1)C(=O)Nc1cnccc12]

The intermediate from step c of this example (3 g, 7.1 mmol) and anisole (3.8 g, 35.4 mmol) were stirred in a solution of 10% triflic acid in TFA (25 mL) at 70° C. for 12 hours (LC/MS [M+H]⁺=204.2). The mixture was cooled and concentrated on the rotary evaporator and azeotroped with toluene (3×). The dark, viscous product mixture was taken up in acetonitrile (50 mL) and cooled to 0° C. The pH was adjusted to 8 by the dropwise addition of DIPEA and boc anhydride (1.5 g, 7.1 mmoL) was added and the reaction was stirred for 40 minutes. The solvent was removed by the rotary evaporator and the crude product mixture was purified by RP HPLC (ISCO COMBI FLASH®, 10-95% acetonitrile in DI water, 0.1% TFA, 40 minute gradient). The pure fractions were pooled and lyophilized to afford the product as a white solid. Yield 69%. LC/MS [M+H]⁺=304.2.

### Synthesis of Int-5A

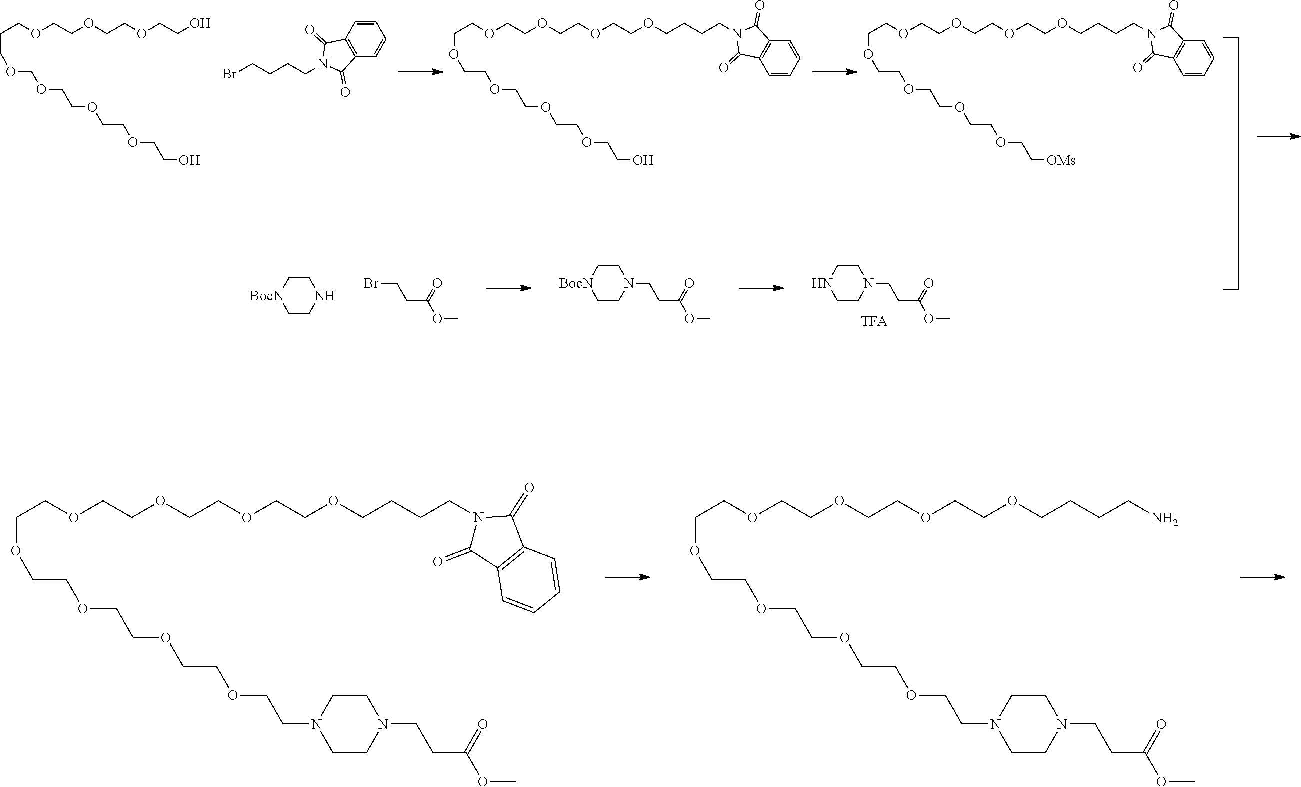

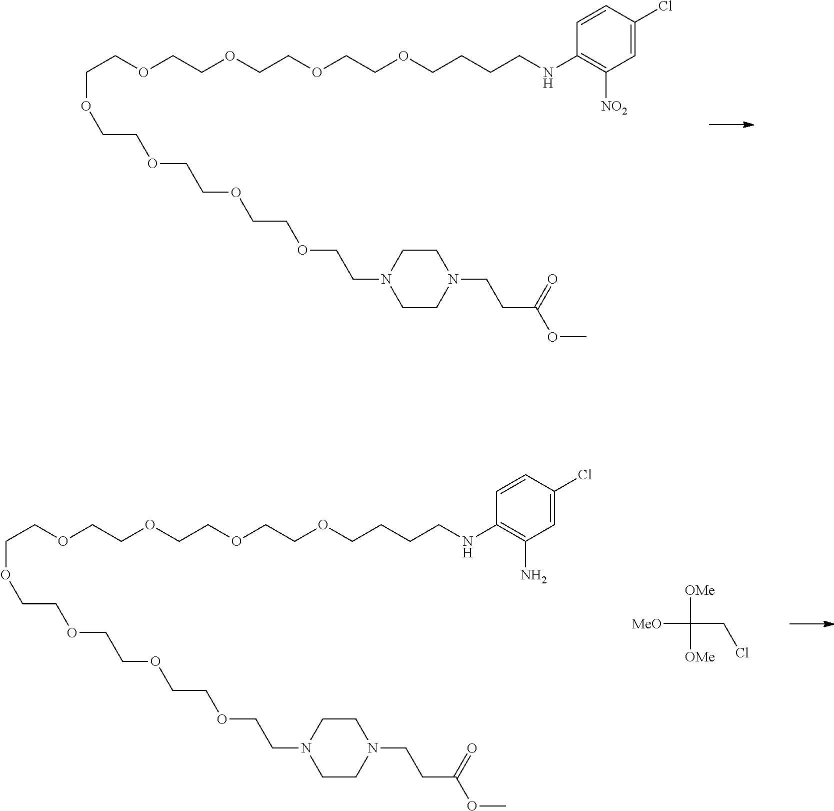 [SMILES: COC(=O)CCN1CCN(CCOCCOCCOCCOCCOCCOCCOCCOCCCCNc2ccc(Cl)cc2[N+](=O)[O-])CC1 ; COC(=O)CCN1CCN(CCOCCOCCOCCOCCOCCOCCOCCOCCCCNc2ccc(Cl)cc2N)CC1 ; COC(CCl)(OC)OC]

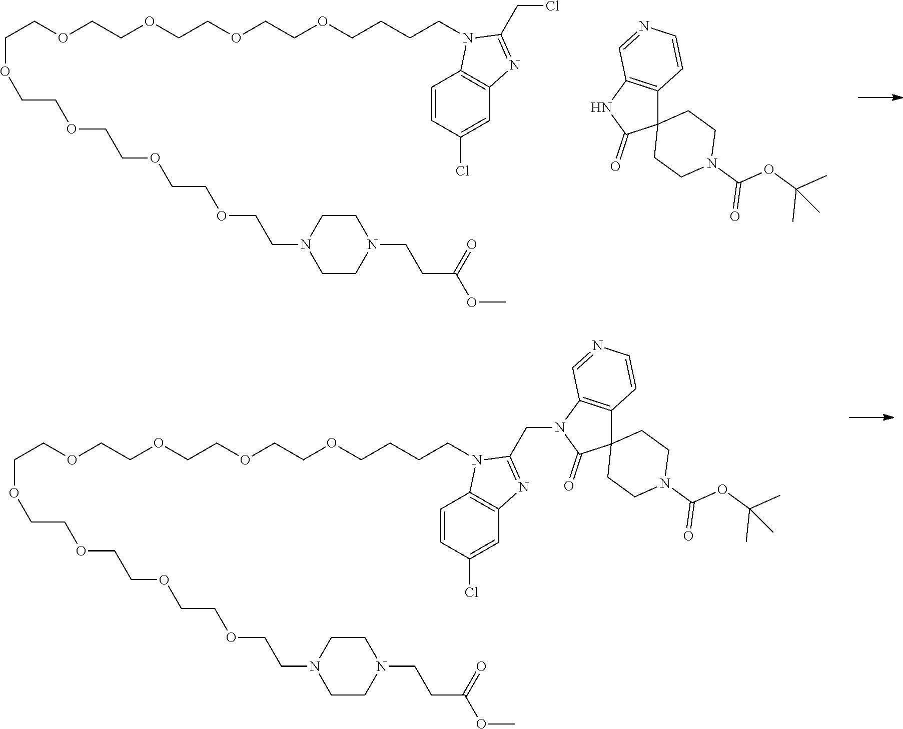 [SMILES: COC(=O)CCN1CCN(CCOCCOCCOCCOCCOCCOCCOCCOCCCCn2c(CCl)nc3cc(Cl)ccc32)CC1 ; CC(C)(C)OC(=O)N1CCC2(CC1)C(=O)Nc1cnccc12 ; COC(=O)CCN1CCN(CCOCCOCCOCCOCCOCCOCCOCCOCCCCn2c(CN3C(=O)C4(CCN(C(=O)OC(C)(C)C)CC4)c4ccncc43)nc3cc(Cl)ccc32)CC1]

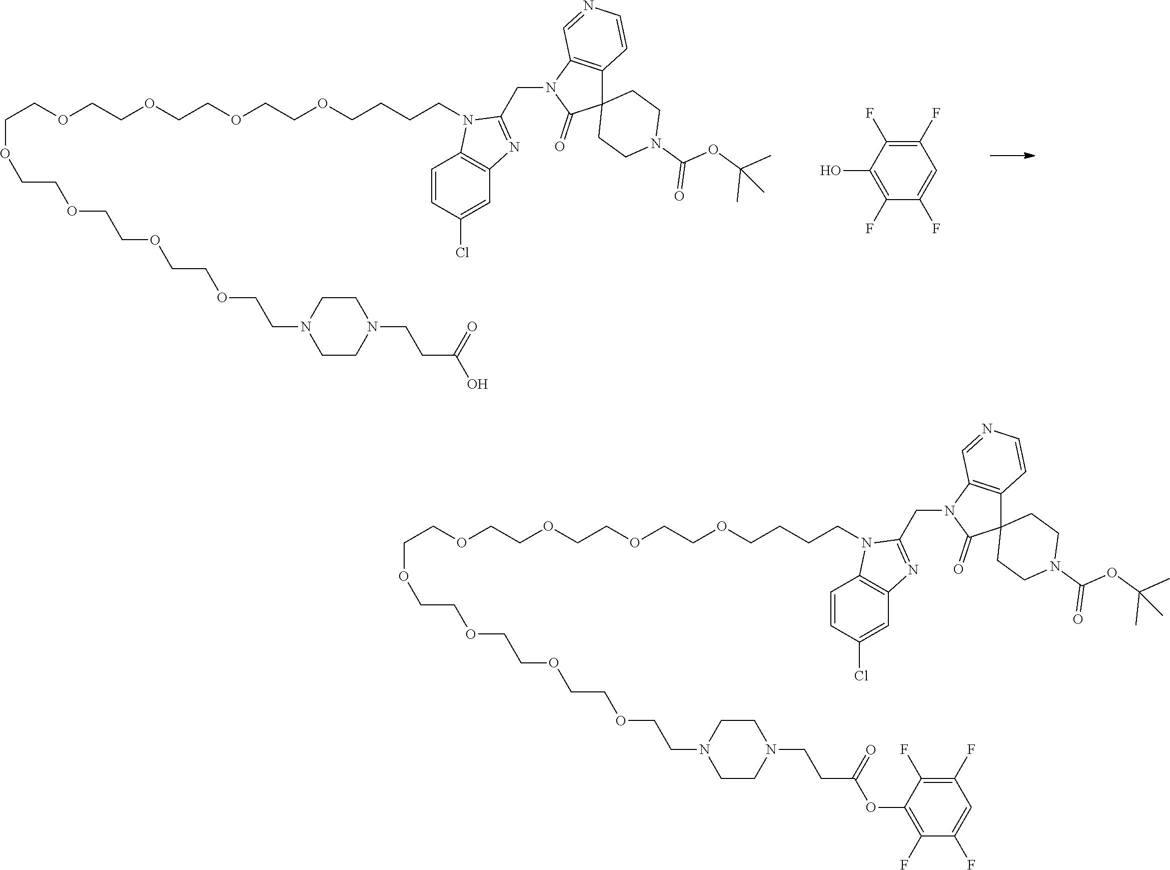 [SMILES: CC(C)(C)OC(=O)N1CCC2(CC1)C(=O)N(Cc1nc3cc(Cl)ccc3n1CCCCOCCOCCOCCOCCOCCOCCOCCOCCN1CCN(CCC(=O)O)CC1)c1cnccc12 ; Oc1c(F)c(F)cc(F)c1F ; CC(C)(C)OC(=O)N1CCC2(CC1)C(=O)N(Cc1nc3cc(Cl)ccc3n1CCCCOCCOCCOCCOCCOCCOCCOCCOCCN1CCN(CCC(=O)Oc3c(F)c(F)cc(F)c3F)CC1)c1cnccc12]

Step a.

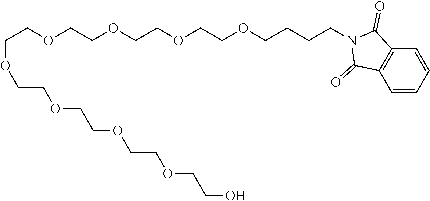 [SMILES: O=C1c2ccccc2C(=O)N1CCCCOCCOCCOCCOCCOCCOCCOCCOCCO]

To a 0° C. solution of octaethylene glycol (5 g, 13.5 mmol) in dry DMF was slowly added sodium hydride (0.32 g, 13.5 mmol, 60% in mineral oil). The solution was stirred on ice bath for 10 mins, and then 2-(4-bromobutyl)isoindoline-1,3-dione (3.8 g, 13.5 mmol) was added. The reaction solution was stirred at room temperature for 16 hours, quenched with tert-butanol (1 ml) and concentrated. The residue was dissolved in DCM (50 ml) and the solution was washed with water (3×, 10 ml), brine (10 ml), then dried over sodium sulfate, filtered and concentrated. The crude product was purified by RPLC (5% to 60% acetonitrile/water, using 0.1% TFA as modifier). Yield 2.78 g, 36%. Ion found by LCMS: [M+H]⁺=572.8.

Step b.

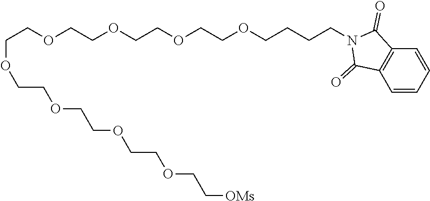 [SMILES: CS(=O)(=O)OCCOCCOCCOCCOCCOCCOCCOCCOCCCCN1C(=O)c2ccccc2C1=O]

To a solution of step-a product (1.78 g, 3.1 mmol) in DCM (20 ml) was added triethylamine (0.86 ml, 6.2 mmol), followed by methanesulfonyl chloride (0.26 ml, 3.43 mmol). The reaction solution was stirred for 2 hours, and then washed with aq HCl (1N, 2×, 5 ml), water (10 ml), brine, and concentrated to give the crude product. Yield 1.9 g, 97%. Ion found by LCMS: [M+H]⁺=649.8.

Step c.

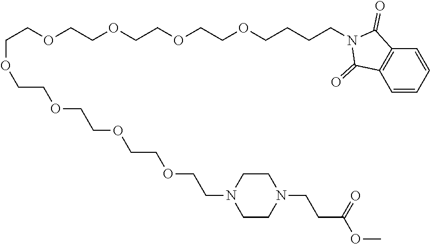 [SMILES: COC(=O)CCN1CCN(CCOCCOCCOCCOCCOCCOCCOCCOCCCCN2C(=O)c3ccccc3C2=O)CC1]

A mixture of step-b product (0.65 g, 1 mmol), methyl 3-(piperazin-1-yl)propanoate (0.63 g, 1 mmol), K₂CO₃ (0.55 g, 4 mmol) in dry acetonitrile (10 ml) were heated at 70° C. overnight. The solution was cooled, filtered, concentrated and purified by RPLC (5% to 90% acetonitrile/water, using 0.1% TFA as modifier). Yield 0.54 g, 74%. Ion found by LCMS: [M+H]⁺=726.8.

Step d.

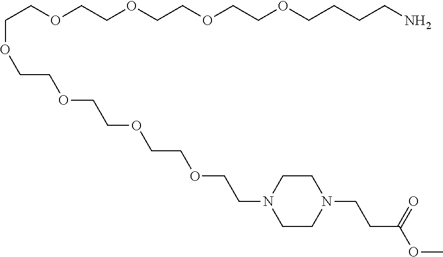 [SMILES: COC(=O)CCN1CCN(CCOCCOCCOCCOCCOCCOCCOCCOCCCCN)CC1]

To a solution of step-c product (600 mg, 0.82 mmol) in EtOH (5 mL) was added hydrazine hydrate (205 mg, 4.1 mmol) and the solution was stirred at 50° C. for 2 hrs. The solution was cooled, filtered, concentrated give the crude product which was used without further purification. Yield 281 mg, 57%, Ion found by LCMS: [M+H]⁺=596.0.

Step e.

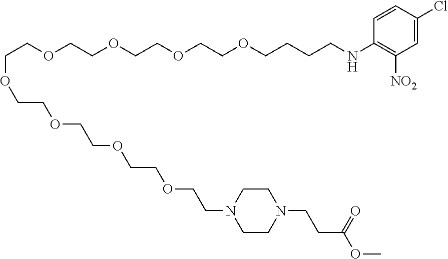 [SMILES: COC(=O)CCN1CCN(CCOCCOCCOCCOCCOCCOCCOCCOCCCCNc2ccc(Cl)cc2[N+](=O)[O-])CC1]

A mixture of step-d product (140 mg, 0.24 mmol), 5-chloro-2-fluoronitrobenzene (165 mg, 0.94 mmol), K2CO3 (100 mg, 0.72 mmol) in dry DMF was heated at 70° C. for 2 hours. The solution was cooled, filtered, concentrated, and purified by RPLC (5% to 40% acetonitrile and water, using 0.1% TFA as modifier). Yield 100 mg, 57%. Ion found by LCMS: [M+H]⁺=751.1.

Step f.

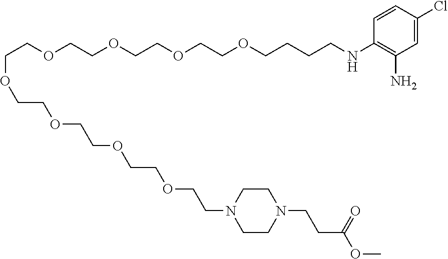 [SMILES: COC(=O)CCN1CCN(CCOCCOCCOCCOCCOCCOCCOCCOCCCCNc2ccc(Cl)cc2N)CC1]

A solution of step-e product (190 mg, 0.25 mmol) in acetic acid (5 ml) was heated at 70° C., and zinc (82 mg, 1.26 mmol) was added cautiously. The solution was stirred at 70° C. for 10 mins at which time the reaction was complete by LCMS. The crude mixture was filtered, and used in the next step without further purification. LC/MS [M+H]⁺=720.8.

Step g.

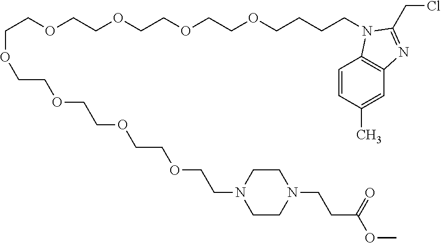 [SMILES: COC(=O)CCN1CCN(CCOCCOCCOCCOCCOCCOCCOCCOCCCCn2c(CCl)nc3cc(C)ccc32)CC1]

To a solution of step-f product in acetic acid (5 ml) was added 2-chloro-1,1,1-trimethoxyethane (300 mg, 1.5 mmol). The reaction was stirred at 70° C. for 1.5 hour. The solution was cooled, concentrated and purified by RPLC (5% to 50% acetonitrile and water, using 0.1% TFA as modifier). Yield 154 mg, 79%. Ion found by LCMS: [M+H]⁺=778.8.

Step h.

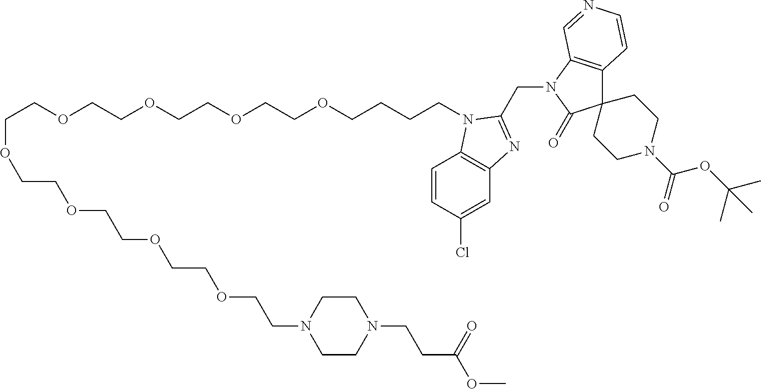 [SMILES: COC(=O)CCN1CCN(CCOCCOCCOCCOCCOCCOCCOCCOCCCCn2c(CN3C(=O)C4(CCN(C(=O)OC(C)(C)C)CC4)c4ccncc43)nc3cc(Cl)ccc32)CC1]

To a solution of Intermediate 6 (tert-butyl 2′-oxo-1′,2′-dihydrospiro[piperidine-4,3′-pyrrolo[2,3-c]pyridine]-1-carboxylate, 53.7 mg, 0.178 mmol) in dry DMF (2 ml) was added Cs₂CO₃ (116 mg, 0.35 mmol), followed by the step-g product (69 mg, 0.088 mmol). The reaction was stirred at r.t for 4 hrs and solution was filtered, concentrated and purified by RPLC (5% to 50% acetonitrile and water, using 0.1% TFA as modifier). Yield 71 mg, 76%. Ion found by LCMS: [M+H]⁺=1046.6.

Step i.

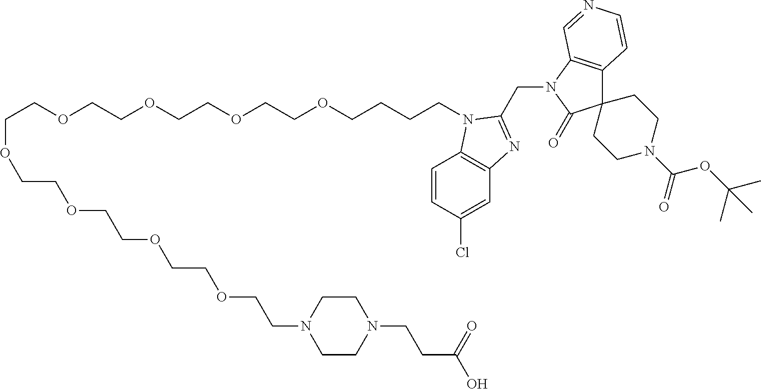 [SMILES: CC(C)(C)OC(=O)N1CCC2(CC1)C(=O)N(Cc1nc3cc(Cl)ccc3n1CCCCOCCOCCOCCOCCOCCOCCOCCOCCN1CCN(CCC(=O)O)CC1)c1cnccc12]

To a solution of step-h product (71 mg, 0.068 mmol) in a solvent mixture of THF:MeOH:H2O (v:v:v=3:1:1) on ice was added LiOH (3.3 mg, 0.14 mmol). The solution was stirred at r.t for 1 hour, concentrated and purified by RPLC (5% to 50% acetonitrile and water, using 0.1% TFA as modifier). Yield 50 mg, 72%. Ion found by LCMS: [M+H]⁺=1031.8.

Step j.

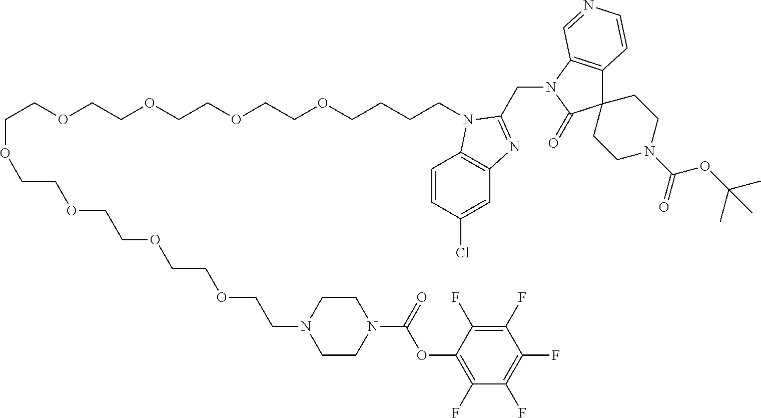 [SMILES: CC(C)(C)OC(=O)N1CCC2(CC1)C(=O)N(Cc1nc3cc(Cl)ccc3n1CCCCOCCOCCOCCOCCOCCOCCOCCOCCN1CCN(C(=O)Oc3c(F)c(F)c(F)c(F)c3F)CC1)c1cnccc12]

To a solution of step-i product (50 mg, 0.049 mmol) in DCM (3 ml) was added EDCI (27 mg, 0.145 mmol) and tetrafluorophenyl (32.5 mg, 0.196 mmol). The solution was then stirred at r.t for 2 hrs, concentrated and purified by ACCQ and RPLC (5% to 50% acetonitrile and water, using 0.1% TFA as modifier). Yield 17.3 mg, 31%. Ion found by LCMS: [M+H]⁺=1180.4.

### Example 9. Synthesis of Int-6C

Int-6C was prepared following the procedure described below.

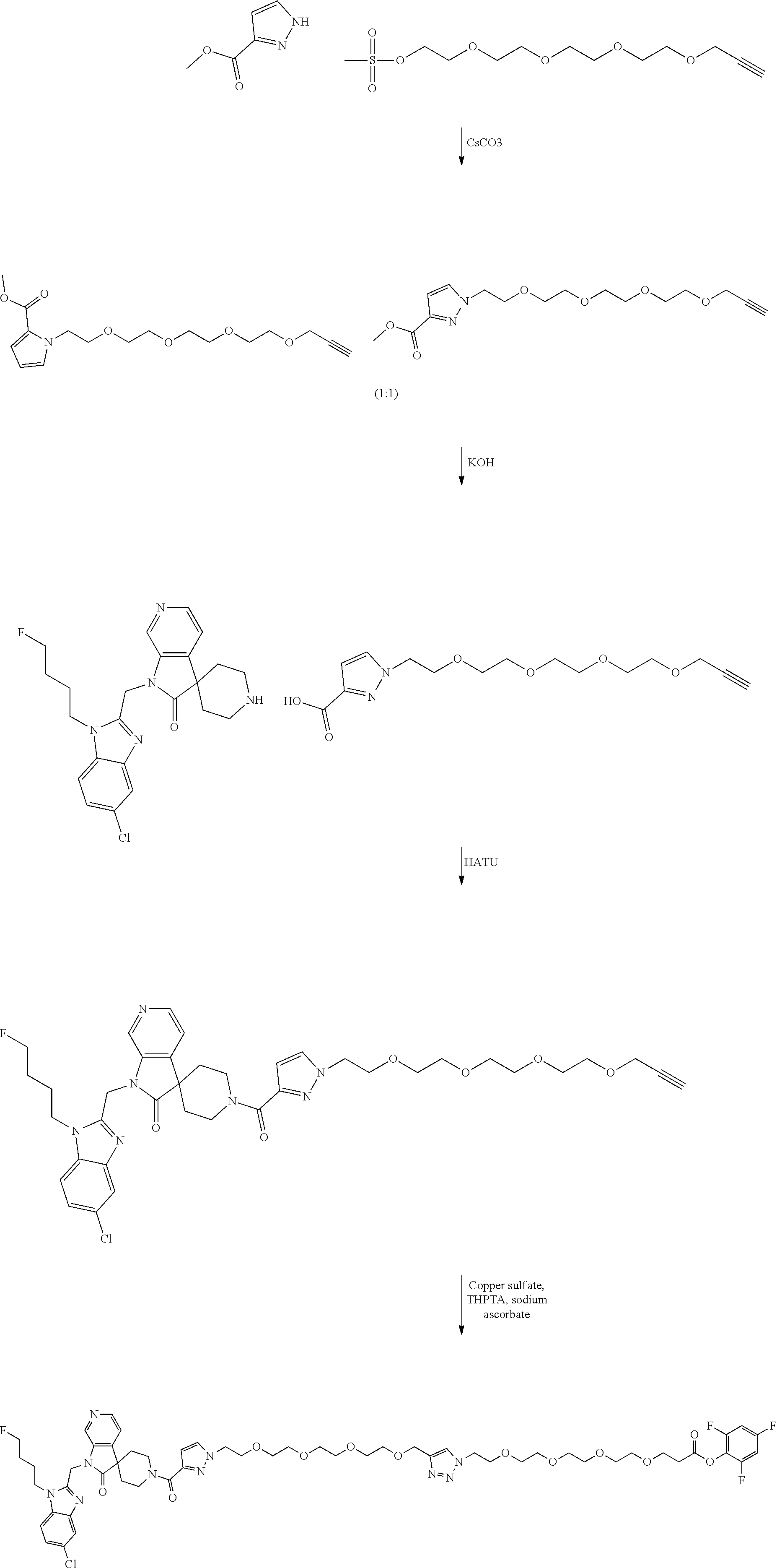

Step a.

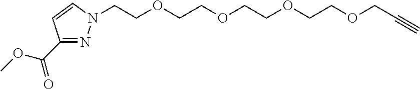 [SMILES: C#CCOCCOCCOCCOCCn1ccc(C(=O)OC)n1]

A solution the pyrazole starting material (305 mg, 2.42 mmol) and propargyl-PEG4-mesylate (0.50 g, 1.61 mmol) dissolved in acetonitrile (8 mL) was treated with cesium carbonate (0.787 g, 2.42 mmol), at room temperature, overnight. After stirring for 12 h LCMS shows complete consumption of start material and formation of a 1:1 mixture of alkylated pyrazole isomers. The isomers were separated by RPLC (5% to 100% acetonitrile/water with 0.1% TFA). The desired isomer was the isomer that first elutes via RPLC. Its structure was determined by analysis of NOE's in the proton NMR. Yield was 0.230 g, 41% yield. LC/MS [M+H]⁺=341.2.

Step b.

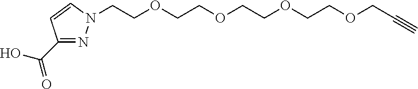 [SMILES: C#CCOCCOCCOCCOCCn1ccc(C(=O)O)n1]

A solution of product from the previous step (0.230 g, 0.676 mmol) was dissolved in MeOH (2.0 mL), and treated with a solution of potassium hydroxide (0.152 g, 2.70 mmol) dissolved in water (2.0 mL). LCMS after 3 h shows complete hydrolysis. The product was acidified with acetic acid and then was loaded directly onto a C18 column and purified by RPLC (10% to 100% acetonitrile/water with 0.1% TFA). Yield 0.232 g, 105%. LC/MS [M+H]⁺=327.2

Step c.

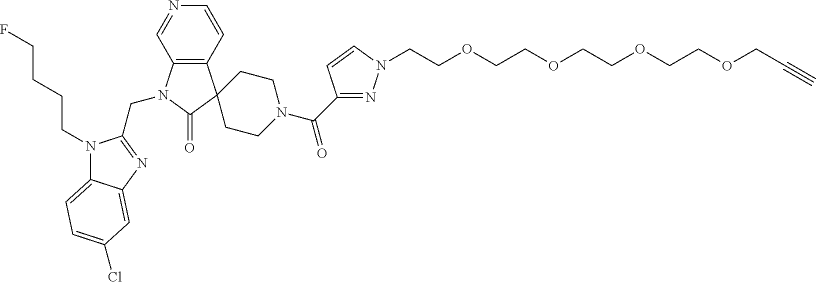 [SMILES: C#CCOCCOCCOCCOCCn1ccc(C(=O)N2CCC3(CC2)C(=O)N(Cc2nc4cc(Cl)ccc4n2CCCCF)c2cnccc23)n1]

A solution of product from the previous step (0.024 g, 0.0747 mmol), previously described piperidine core (described in WO 2015158653, incorporated herein in its entirety) (0.030 g, 0.068 mmol) and diisopropylethylamine (0.071 mL, 0.407 mmol) were dissolved in DMF (100 μL), and treated with HATU (0.034 g, 0.088 mmol) at room temperature. LCMS after 10 min shows complete conversion. The crude reaction was loaded directly onto a C18 column and purified by RPLC (10% to 100% acetonitrile/water with 0.1% TFA). Yield 0.033 g, 56%. LC/MS [M+H]⁺=749.8

Step d.

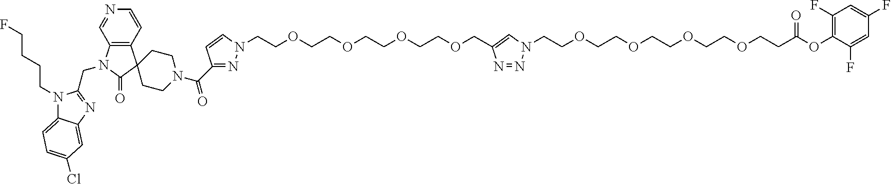 [SMILES: O=C(CCOCCOCCOCCOCCn1cc(COCCOCCOCCOCCn2ccc(C(=O)N3CCC4(CC3)C(=O)N(Cc3nc5cc(Cl)ccc5n3CCCCF)c3cnccc34)n2)nn1)Oc1c(F)cc(F)cc1F]

A solution of product from the previous step (0.030 g, 0.0400 mmol), and 1-[15-oxo-15-(2,4,6-trifluorophenoxy)-3,6,9,12-tetraoxapentadecan-1-yl]azide (0.020 g, 0.048 mmol), dissolved in DMF (750 μL), was treated with a solution of THPTA (0.0069 g, 0.016 mmol), sodium ascorbate (0.0079 g, 0.040 mmol), and copper sulfate (0.0016 g, 0.010 mmol), dissolved in water (750 μL) at room temperature. LCMS after 20 min shows complete conversion. The crude reaction was loaded directly onto a C18 column and purified by RPLC (5% to 100% acetonitrile/water with 0.1% TFA). Product containing fractions were pooled and dried by rotary evaporation. Yield 0.035 g, 68%. LC/MS [(M+2H)/2]⁺=586.2

### Example 10. Synthesis of Conjugate Using Tetrafluorophenyl Ester Intermediate

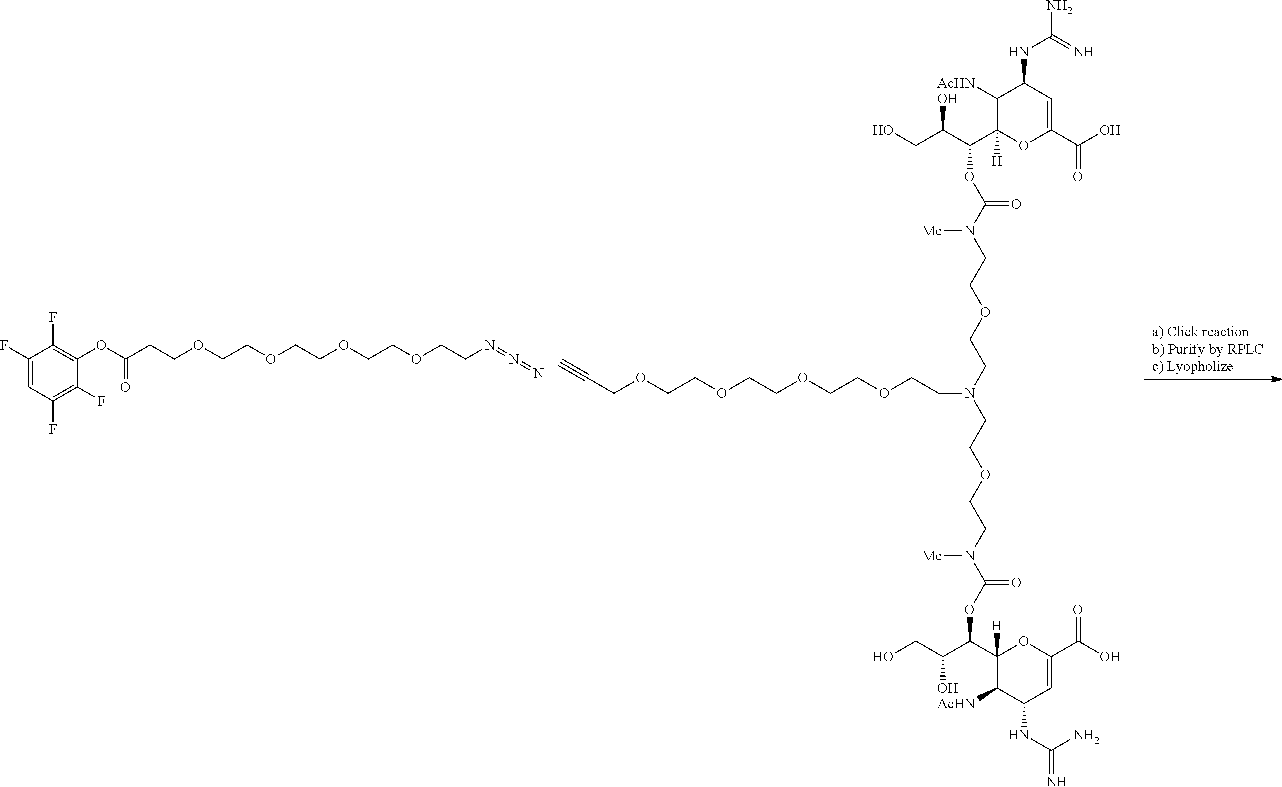

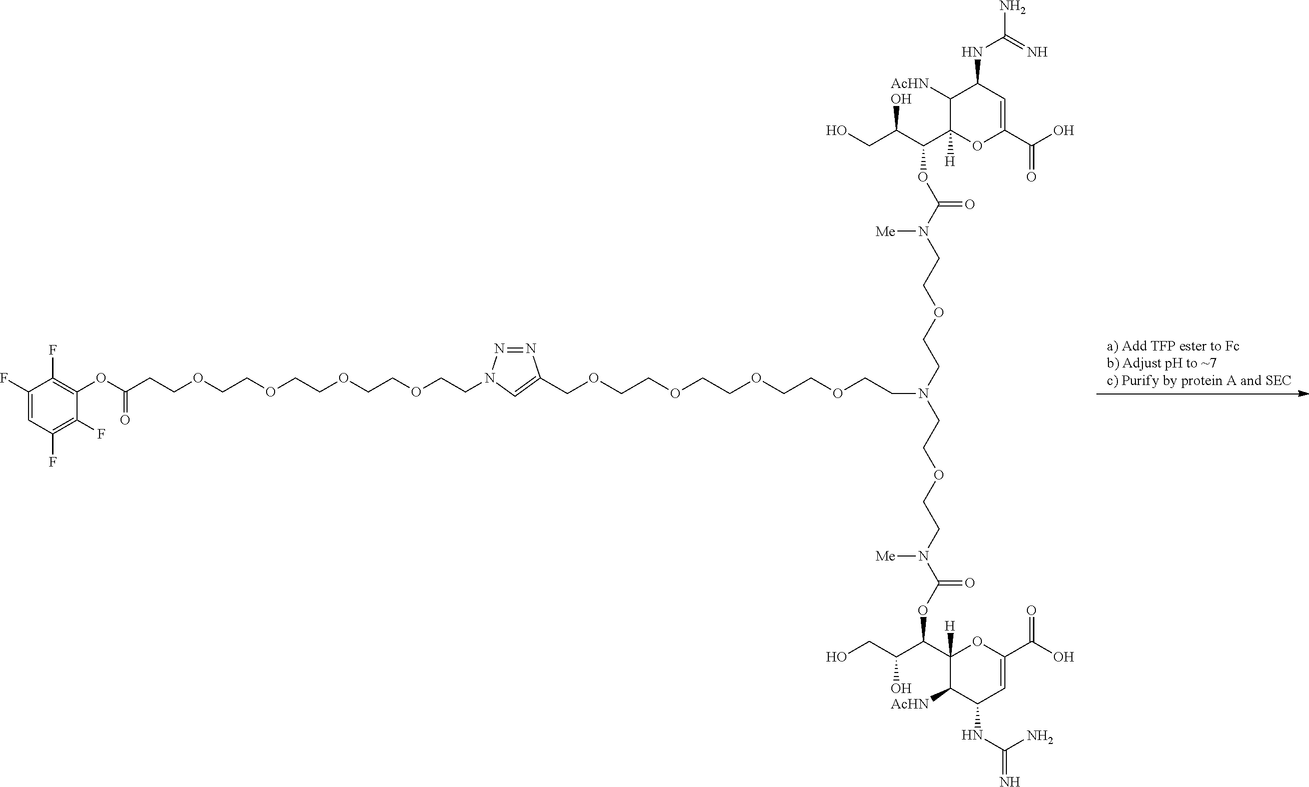 [SMILES: CC(=O)NC1[C@@H](NC(=N)N)C=C(C(=O)O)O[C@H]1[C@H](OC(=O)N(C)CCOCCN(CCOCCOCCOCCOCc1cn(CCOCCOCCOCCOCCC(=O)Oc2c(F)c(F)cc(F)c2F)nn1)CCOCCN(C)C(=O)O[C@@H]([C@@H]1OC(C(=O)O)=C[C@H](NC(=N)N)[C@H]1NC(C)=O)[C@H](O)CO)[C@H](O)CO]

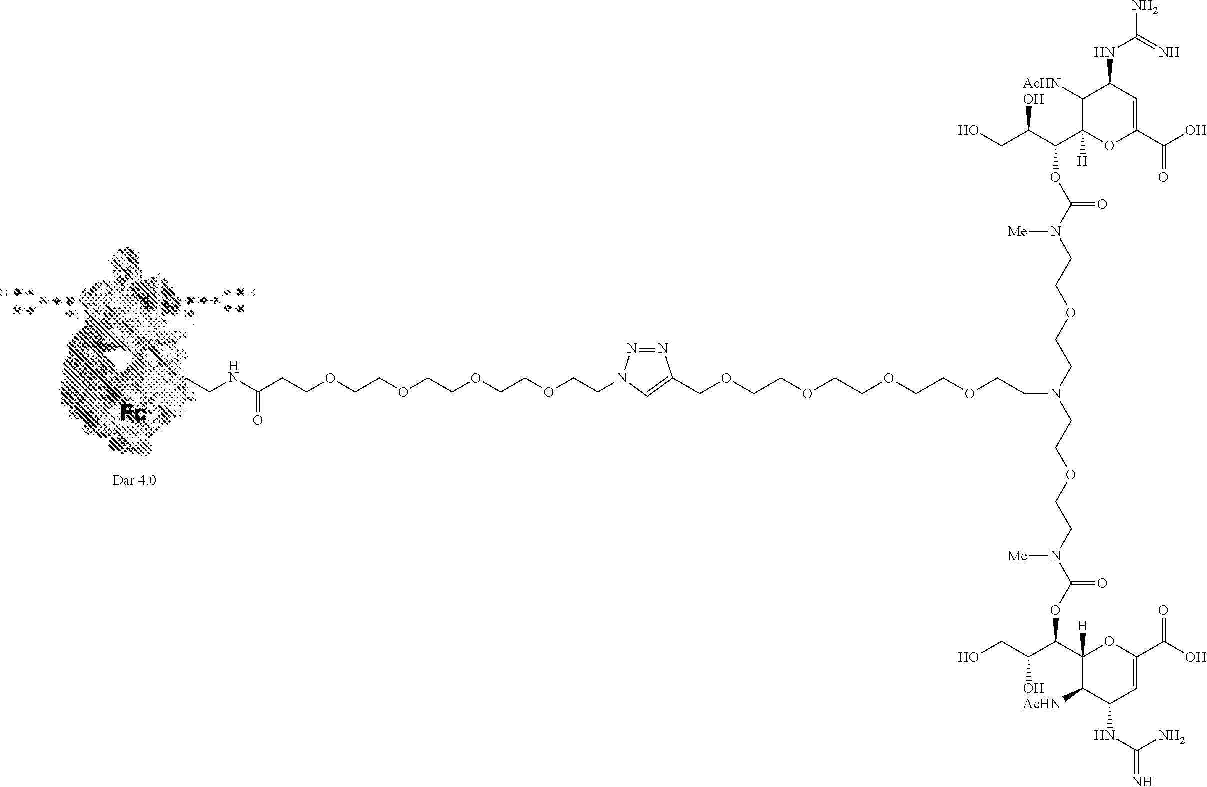 [SMILES: CCNC(=O)CCOCCOCCOCCOCCn1cc(COCCOCCOCCOCCN(CCOCCN(C)C(=O)O[C@H]([C@H](O)CO)[C@@H]2OC(C(=O)O)=C[C@H](NC(=N)N)C2NC(C)=O)CCOCCN(C)C(=O)O[C@@H]([C@@H]2OC(C(=O)O)=C[C@H](NC(=N)N)[C@H]2NC(C)=O)[C@H](O)CO)nn1]

Step a.

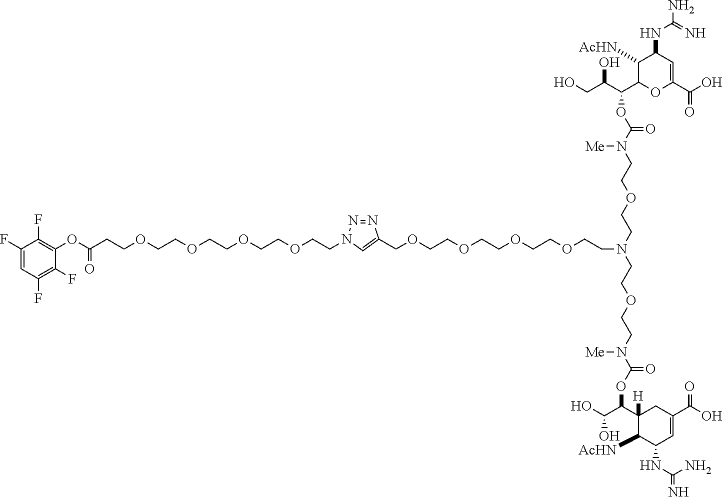

A solution of azido-PEG4-TFP ester (0.1 g, 0.067 mmol) and alkyne functionalized dimer (0.0383 g, 0.0871 mmol) in DMF (2.0 mL), were treated with a solution of copper(II)sulfate (0.0027 g, 0.0168 mmol), sodium ascorbate (0.0133 g, 0.067 mmol), and THPTA (0.0116 g, 0.027 mmol) at room temperature, in water (1.5 mL). The reaction was then vacuum flushed with nitrogen 3× and stirred under an atmosphere of nitrogen. LCMS after 30 min shows complete consumption of starting material. The reaction was acidified with 400 μL of acetic acid, and then purified directly by reverse phase chromatography eluting with a gradient of 5% to 100% acetonitrile/water with 0.1% TFA. The product containing fractions were combined, frozen, and lyophilized overnight. Yield of triple TFA salt was 69%. Ion(s) found by LCMS: (M+2H)⁺²=795.4, (M+3H)⁺³=530.8, (M+4H)⁺⁴=398.4.

Step b.

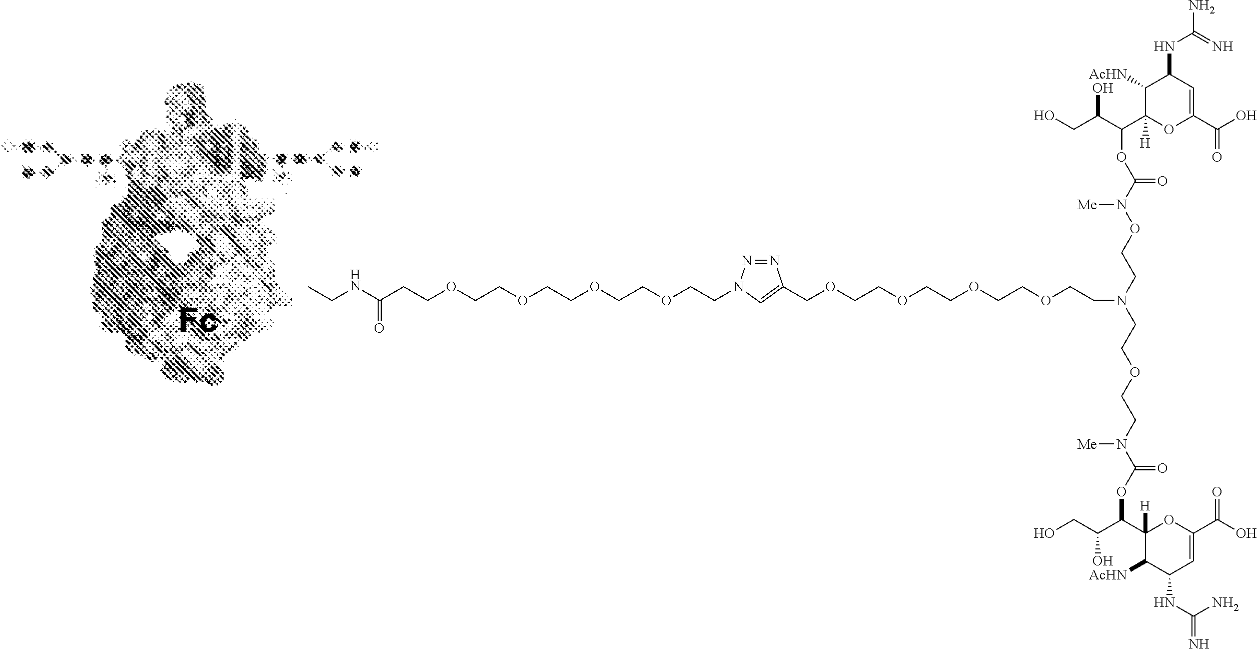

A solution of polypeptide having sequence of SEQ ID NO: 2 (0.100 g in 5.2 mL, 1.717 μmol, MW=58,218) in pH=7.4 PBS buffer was treated with solid TFP ester (0.0273 g, 17.17 μmol) from the previous step. The pH was adjusted to ˜7.0 with borate buffer (120 μL, 1M, pH 8.5) then was gently rocked at room temperature. Maldi TOF after 1.5 h shows an average DAR of 3.3, which did not change upon further mixing. After 24 hr additional TFP ester (0.0073 g, 4.6 μmol) was added and rocking was continued for another 3 h. The crude conjugate was purified Protein A and SEC according to general purification methods. Total yield after Protein A was ˜83%, and after SEC˜77%. Maldi TOF of the purified conjugate showed an average mass of 63,574, which equates to an average DAR of 4.0.

The synthesis described in this example and other examples is advantageous at it avoids exposing the polypeptide to copper+2 and sodium ascorbate, leading to a cleaner crude conjugate that is 98.9% pure by analytical SEC after protein A purification alone. At this level of purity, it may be possible to eliminate the SEC purification which is time very consuming and costly. Initial by attempts with an azido-PEG4-NHS ester were only partially successful because the NHS ester is too reactive to be purified, and the crude click reaction mixture had to be mixed with the Fc, thus necessitating copper removal and high molecular weight aggregate removal (from exposure to sodium ascorbate).

### Example 11. Synthesis of Conjugate Using Trifluorophenyl Ester Intermediate

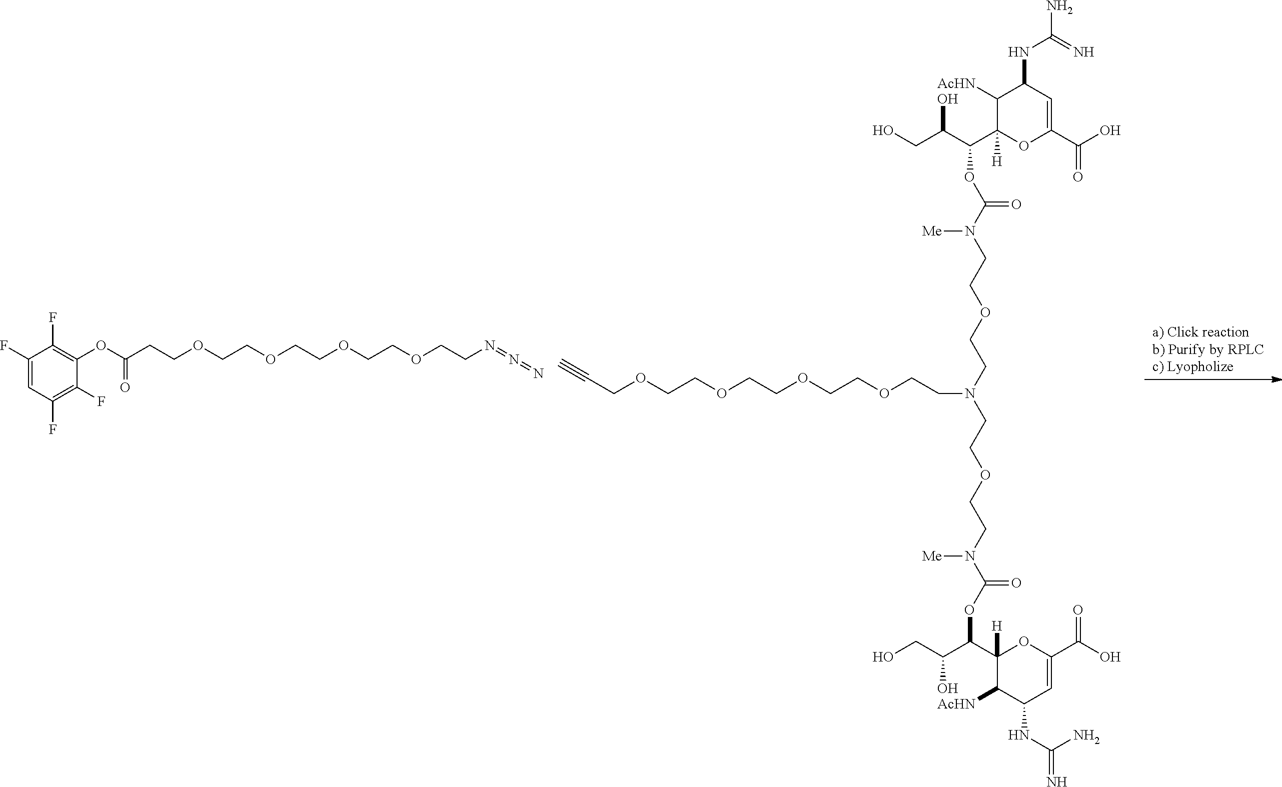

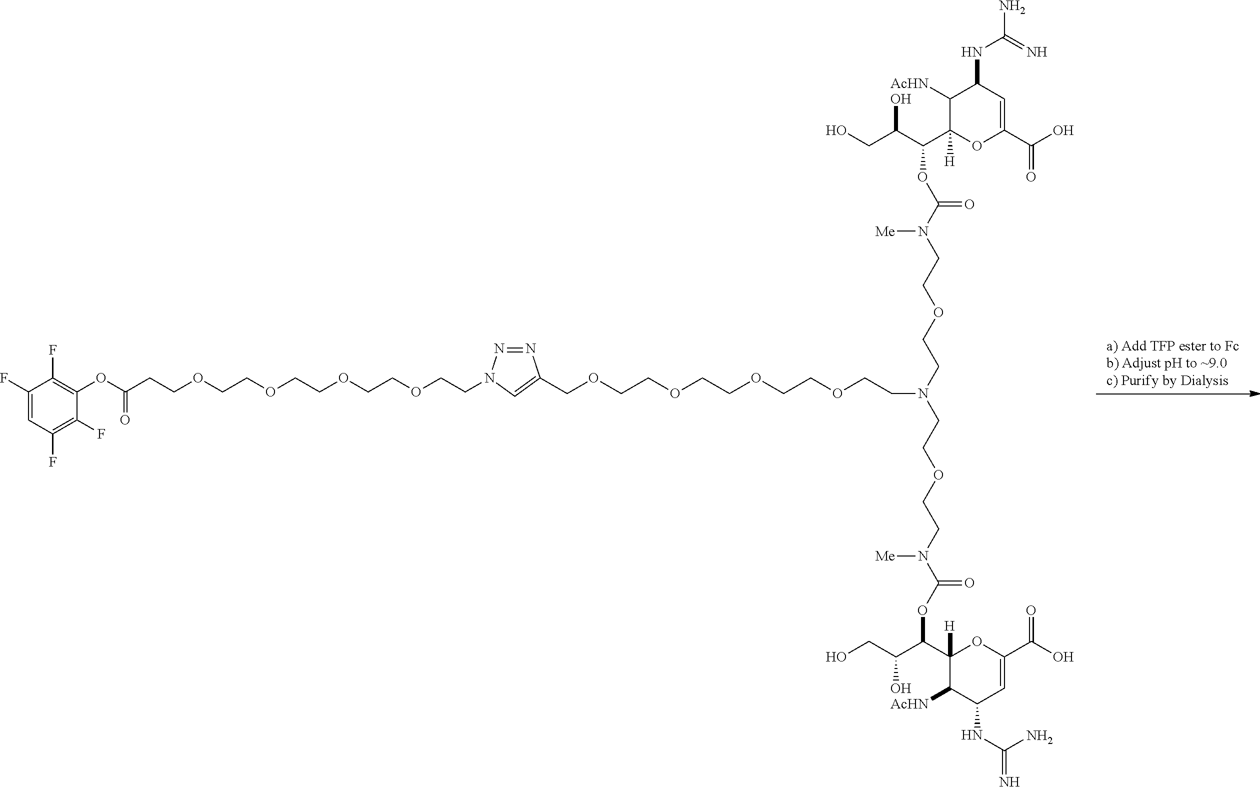

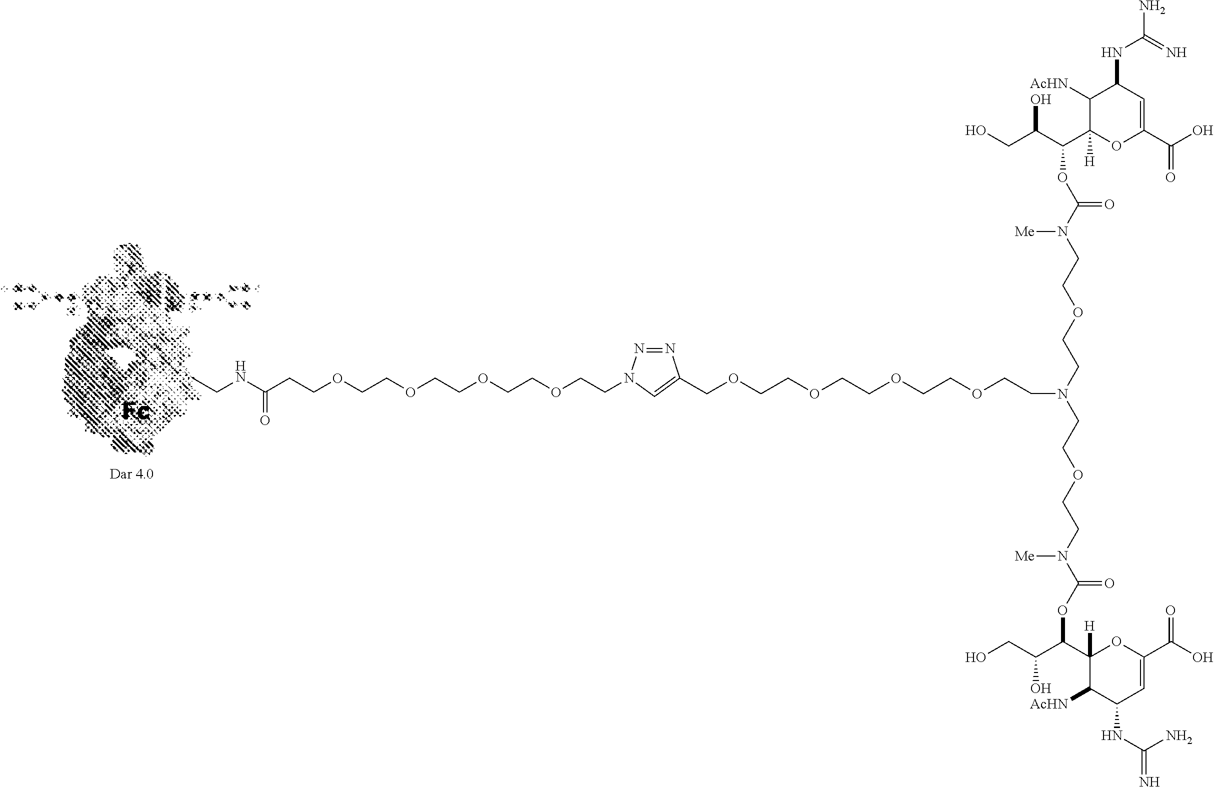

Step a.

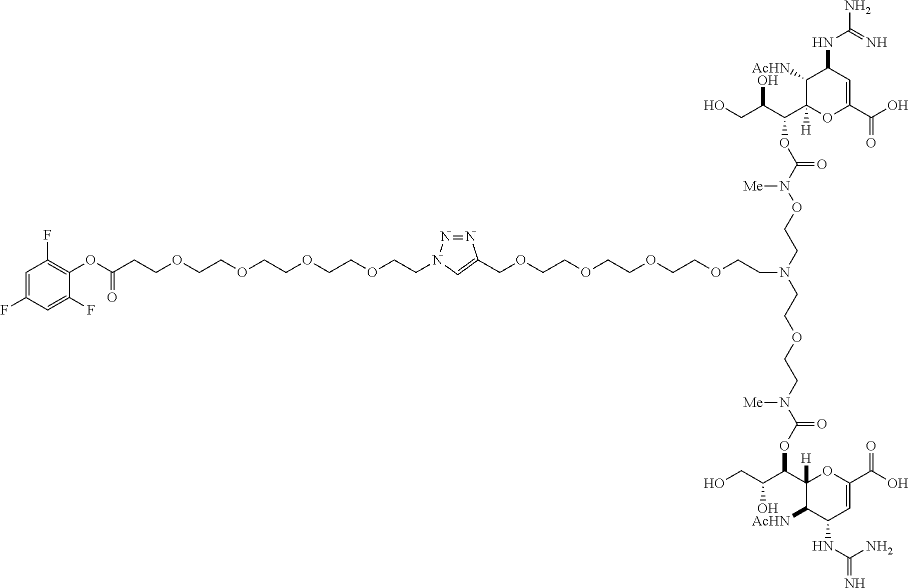

A solution of azido-PEG4-TriFP ester (0.405 g, 0.96 mmol) and alkyne functionalized dimer (0.850 g, 0.74 mmol) in DMF (4.0 mL), were cooled to 0° C. To this solution was added a solution of copper(II)sulfate (0.030 g, 0.18 mmol) and sodium ascorbate (0.146 g, 0.74 mmol), in water (4.0 mL). The reaction was then vacuum flushed with nitrogen 3× and stirred under an atmosphere of nitrogen. LCMS after 30 min shows complete consumption of starting material. The reaction was acidified with acetic acid (0.1 mL, 1.75 mmol), and then purified directly by reversed phase chromatography eluting with a gradient of 0% to 80% acetonitrile/water with 0.1% TFA. The product containing fractions were combined frozen, and lyophilized. Yield of triple TFA salt was 65%, 920 mg. Ion(s) found by LCMS: (M+2H)⁺²=786.4, (M+3H)⁺³=524.8, (M+4H)⁺⁴=393.8.

Step b.

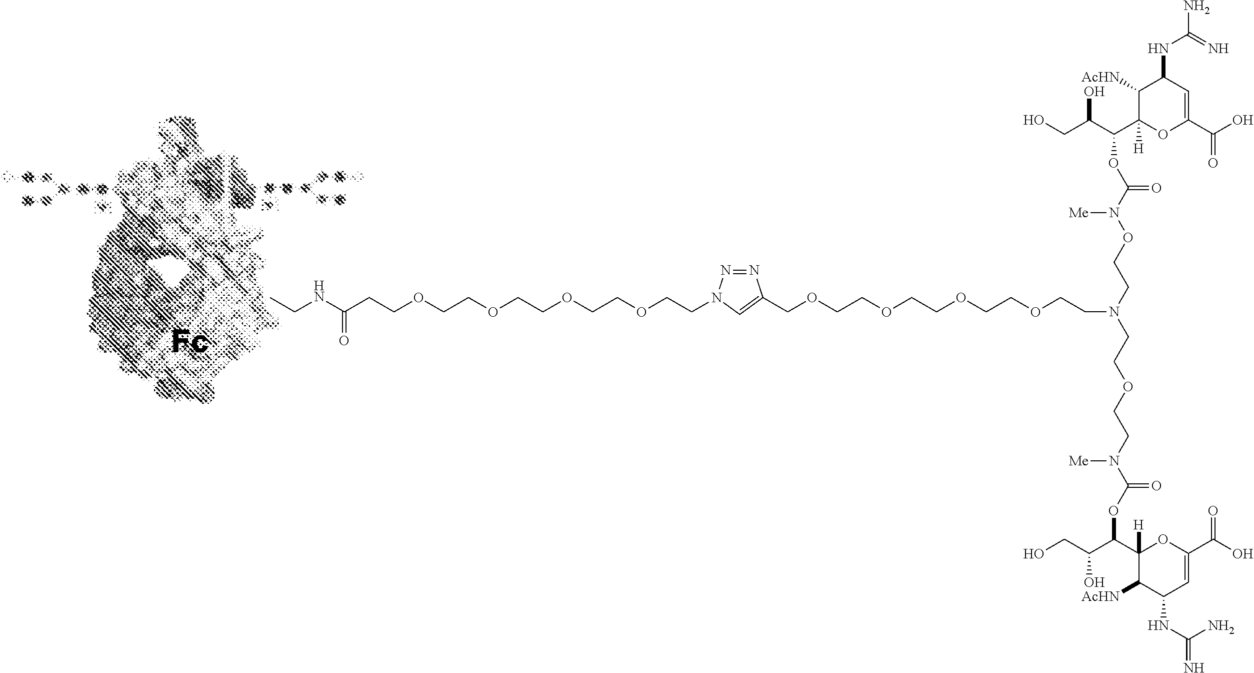

A solution of polypeptide having sequence of SEQ ID NO: 5 (2.0 g in 100 mL, 0.034 mmol, MW=58,200, YTE) in acetate buffer at pH 5.0 was treated with carbonate buffer (pH 9.5, 0.1M, 24-30 mL) to adjust the requisite pH to 9.0. Solid TFP ester (0.710 g, 0.39 mmol) from the previous step was then added at which point the pH decreased back to 6.0-7.0. The pH was again adjusted to ˜9.0-9.5 with the carbonate buffer (12-18 mL). The solution was then gently rocked at room temperature for 3 h. Maldi TOF after 1.5 hours shows an average DAR of 3.5-4.0. After an additional 1 h the DAR had risen to 4.4-4.6 and the reaction was quenched with the addition of concentrated NH₄OH (0.100 mL). The crude conjugate was dialyzed with the following buffer: 120 mM NaCl, 250 mM Arginine, 0.1% sucrose pH 6 buffer. Total yield was ˜80%. Maldi TOF of the purified conjugate showed an average mass of 64,724, which equates to an average DAR of 4.6.

Trifluorophenyl ester compounds (e.g., the compound resulting from step a of this example or compounds of formula (F-I), (F-II), (F-II-A), (F-II-B), (G1-A), and (G2-A)) can provide further advantages in the synthesis of protein-drug conjugates. For example, trifluorophenyl ester compounds can exhibit increased stability, which allows for, e.g., purification by reverse phase chromatography and lyophilization with minimal hydrolysis of the activated ester.

### Example 12. Synthesis of Conjugates Using Alkyne Intermediates

The following conjugates were prepared using an alternative synthetic method including the use of click chemistry to conjugate an alkyne intermediate with a polypeptide functionalized with an azido group.

### Synthesis of Azido Polypeptide

Preparation of PEG4-azido NHS ester solution (0.050 M) in DMF/PBS-PEG4-azido NHS ester (16.75 mg) was dissolved in DMF (0.100 mL) at 0° C. and diluted to 0.837 mL by adding PBS 1× buffer at 0° C. This solution was used for preparing other PEG4-azido Fc with a variety of DAR values by adjusting the equivalents of this PEG4-azido NHS ester PBS solution.

Pretreatment of polypeptide (SEQ ID NO: 2)—The polypeptide solution was transferred into four centrifugal concentrators (30,000 MWCO, 15 mL) and diluted to 15 mL with PBS×1 buffer and concentrated to a volume of ˜1.5 mL. The residue was diluted 1:10 in PBS pH 7.4, and concentrated again. This wash procedure was repeated for total of four times followed by dilution to 8.80 mL.

Preparation of PEG4-azido polypeptide—The 0.050M PEG4-azidoNHS ester PBS buffer solution (0.593 mL, 29.6 μmol, 16 equivalents) was added to above solution of polypeptide (SEQ ID NO: 2) and the mixture was shaken rotated for 2 hours at ambient temperature. The solution was concentrated by using four centrifugal concentrators (30,000 MWCO, 15 mL) to a volume of ˜1.5 mL. The crude mixture was diluted 1:10 in PBS pH 7.4, and concentrated again. This wash procedure was repeated for total of three times. The concentrated polypeptide-PEG4-azide was diluted to 8.80 mL with pH 7.4 PBS buffer and ready for Click conjugation. The purified material was quantified using a NANODROP™ UV visible spectrophotometer (using a calculated extinction coefficient based on the amino acid sequence of h-IgG1). Yield was quantitative after purification.

### Conjugate 12A

### Synthesis of Int-12A

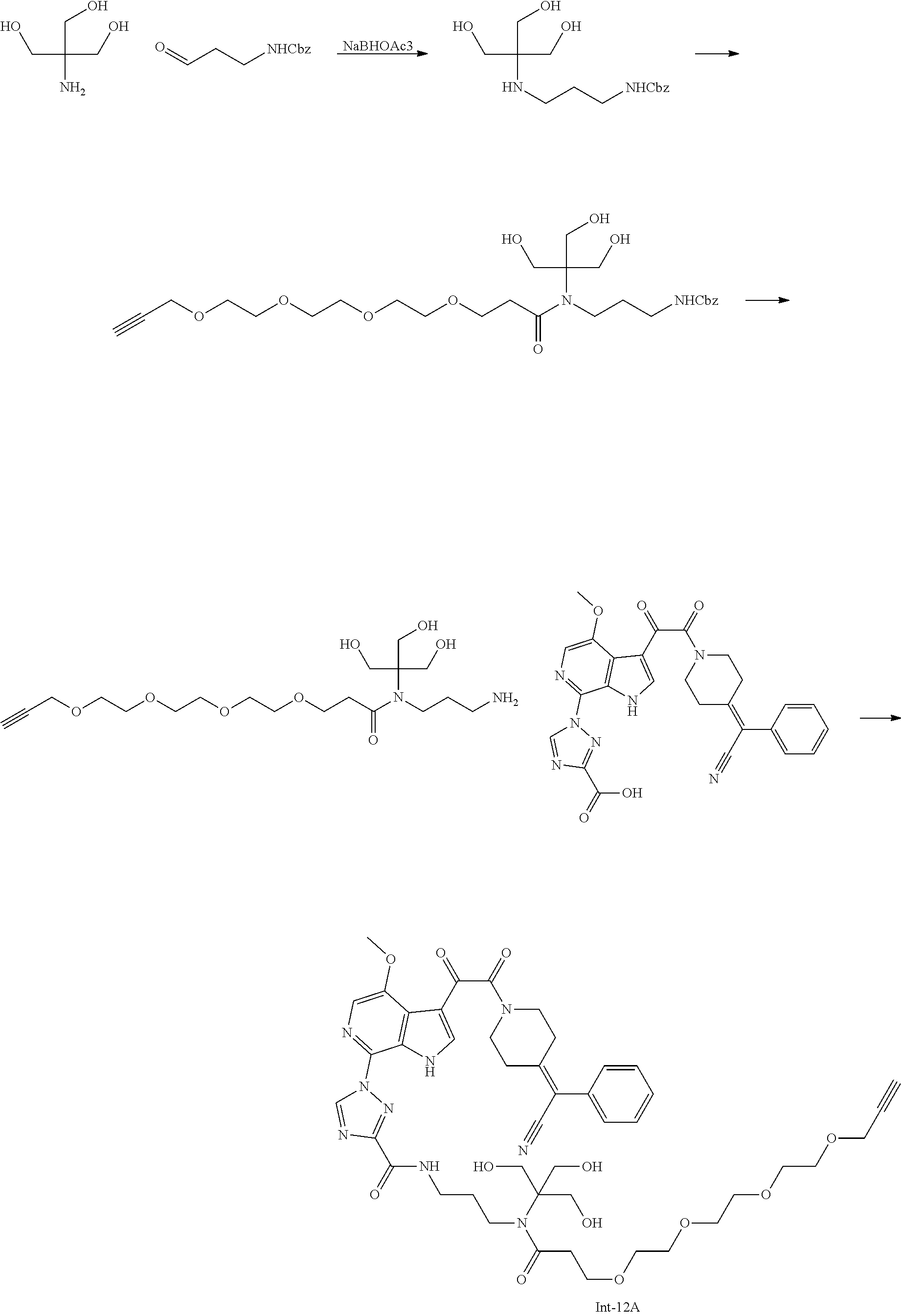

Step a.

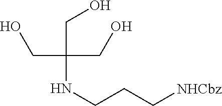 [SMILES: O=C(NCCCNC(CO)(CO)CO)OCc1ccccc1]

To a solution of Tris(hydroxymethyl)-aminomethane (1.22 g, 10 mmol) and 3-[(Benzyloxycarbonyl)amino]-1-propanal (2.1 g, 10 mmol) in DCM (20 mL) and methanol (10 ml) was added acetic acid (1 ml). The resulting solution was stirred for 1 hour at room temperature, then treated under vigorous stirring with sodium triacetoxyborohydride (4.2 g, 20 mmol). This mixture was stirred overnight, then concentrated and purified by reverse phase liquid chromatography (RPLC) using an Isco CombiFlash liquid chromatograph eluted with 5% to 80% acetonitrile and water with 0.1% TFA as modifier. Yield of the products 2.3 g, 72.0%. Ion(s) found by LCMS: M+H=313.2.

Step b.

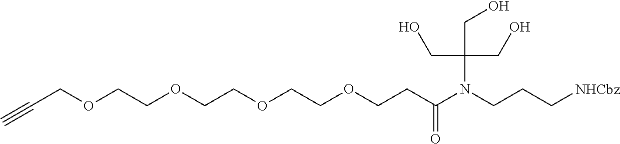 [SMILES: C#CCOCCOCCOCCOCCC(=O)N(CCCNC(=O)OCc1ccccc1)C(CO)(CO)CO]

To a solution of the product from the previous step (0.1 g, 0.32 mmol) and propargyl-PEG4-acid (130 mg, 0.5 mmol) in DMF (5 ml) was added HATU (38 mg, 0.1 mmol), and N-methylmorpholine (0.14 ml, 1 mmol) at room temperature, and the resulting solution was stirred for 1 hour at room temperature. The solution was concentrated and purified by and purified by reverse phase liquid chromatography (RPLC) using an Isco CombiFlash liquid chromatograph eluted with 10% to 100% acetonitrile and water with 0.1% TFA as modifier. Yield of products 120 mg, 68%. Ion(s) found by LCMS: M+H=554.3.

Step c.

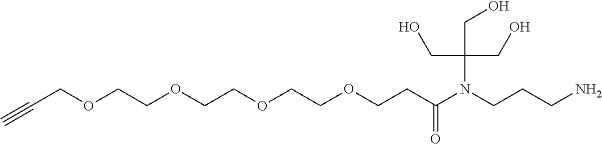 [SMILES: C#CCOCCOCCOCCOCCC(=O)N(CCCN)C(CO)(CO)CO]

The product from the previous step (0.2 g, 32 mmol) was treated with TFA (3 mL) and thioanisole (0.2 ml), and the resulted solution was heated to 45° C. for overnight. The solution was concentrated and purified by and purified by reverse phase liquid chromatography (RPLC) using an Isco CombiFlash liquid chromatograph eluted with 10% to 100% acetonitrile and water with 0.1% TFA as modifier. Yield was quantitative for this step. Ion(s) found by LCMS: M+H=421.3.

Step d.

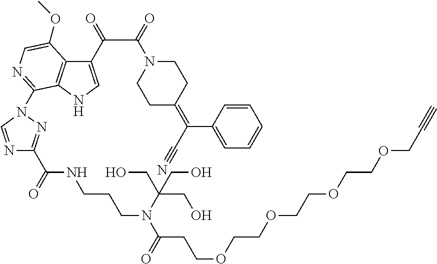 [SMILES: C#CCOCCOCCOCCOCCC(=O)N(CCCNC(=O)c1ncn(-c2ncc(OC)c3c(C(=O)C(=O)N4CCC(=C(C#N)c5ccccc5)CC4)c[nH]c23)n1)C(CO)(CO)CO]

To a solution of 1-{3-[{4-[cyano(phenyl)methylidene]piperidin-1-yl}(oxo)acetyl]-4-methoxy-1H-pyrrolo[2,3-c]pyridine-7-yl}-1H-1,2,4-triazole-3-carboxylic acid (50 mg, 0.1 mmol, described in Example 5, Int-2 and the product from previous step (41 mg, 0.1 mmol) in DMF (2 ml) was added HATU (38 mg, 0.1 mmol), and N-Methylmorpholine (0.07 ml, 0.5 mmol) at room temperature, and the resulting solution was stirred for 1 hour at room temperature. The solution was concentrated and purified by and purified by reverse phase liquid chromatography (RPLC) using an Isco CombiFlash liquid chromatograph eluted with 10% to 100% acetonitrile and water with 0.1% TFA as modifier. Yield of product 21 mg, 24.0%. Ion(s) found by LCMS: M+H=914.4.

Click Conjugation

A preparation of 0.0050M CuSO₄ in PBS buffer solution Click reagent was performed. Briefly, 10.0 mg CuSO₄ was dissolved in 12.53 mL PBS, next 6.00 mL of the CuSO₄ solution and added 51.7 mg BTTAA (CAS #1334179-85-9) and 297.2 mg sodium ascorbate to give the Click reagent solution (0.0050M CuSO4, 0.020M BTTAA and 0.25M sodium ascorbate).

A solution of azido functionalized polypeptide was added to a 15 mL centrifuge tube containing alkyne intermediate, Int-12A (2 equivalents for each DAR). After gently shaking to dissolve all solids, the mixture was treated with the Click reagent solution of (L-ascorbic acid sodium, 0.25 M, 400 equivalents, copper (II) sulfate 0.0050M, 8 equivalents, and BTTAA 0.020M, 32 equivalents). The resulting mixture was gently rotated for 6 hours at ambient temperature. It was purified by affinity chromatography over a protein A column, followed size exclusion chromatography (as described herein). Yield=35%. Maldi-TOF=60,152. DAR=1.6.

### Conjugate 12B

### Synthesis of tert-butyl 2′-oxo-1′,2′-dihydrospiro[piperidine-4,3′-pyrrolo[2,3-c]pyridine]-1-carboxylate

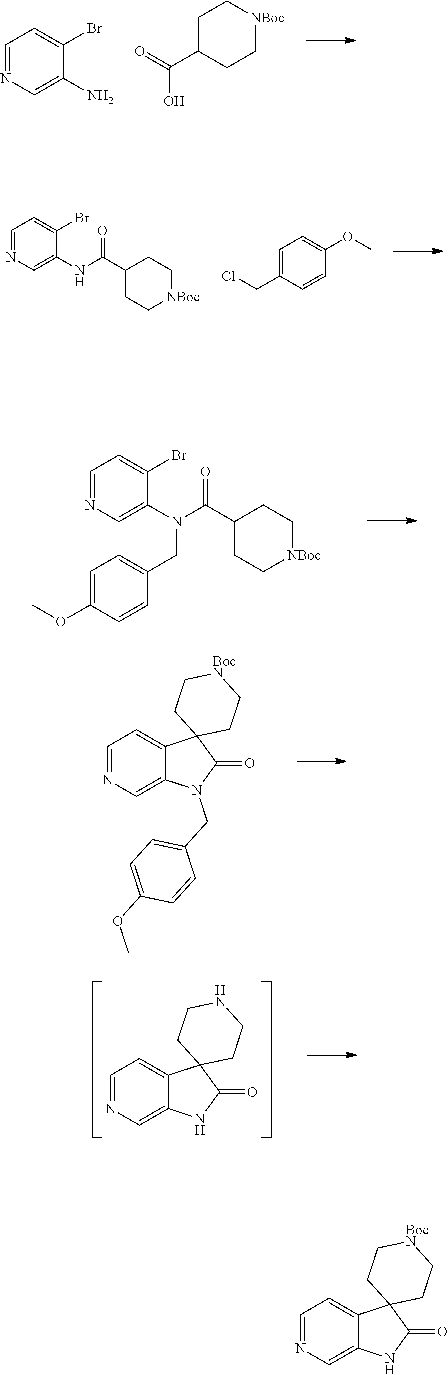

Step a.

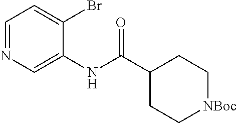 [SMILES: CC(C)(C)OC(=O)N1CCC(C(=O)Nc2cnccc2Br)CC1]

T3P (41.6 mL, 69.9 mmol, 50% by wt. in ethyl acetate) was added, dropwise over 10 minutes, to a stirring mixture of 2-amino-2-bromo-pyridine (11 g, 63.6 mmol), N-Boc-piperazine carboxylic acid (16 g, 69.9 mmol), and DIPEA (16.4 g, 127.2 mmoL) in ethyl acetated (75 mL) cooled to 0° C. The ice bath was removed and the reaction was stirred for 24 hours. The reaction mixture was diluted with water, extracted into ethyl acetate (3×, 25 mL). The combined organic extracts were dried over sodium sulfate, and concentrated on the rotary evaporator. The crude product was by silica gel chromatography on the ISCO COMBI FLASH® (15% to 100% ethyl acetate in hexanes, 25 minutes). The pure fractions were pooled and concentrated to afford the intermediate as a light orange oil. Yield 61%. LC/MS [M+H]⁺=384.2.

Step b.

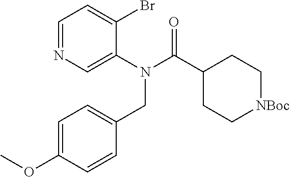 [SMILES: COc1ccc(CN(C(=O)C2CCN(C(=O)OC(C)(C)C)CC2)c2cnccc2Br)cc1]

p-Methoxy benzyl chloride (11.6 g, 74.2 mmol) was added to a mixture of the intermediate from step a. of this example (19.1 g, 49.4 mmol) and cesium carbonate (24.1 g, 74.1 mmol) in DMF (30 mL). The reaction was stirred at room temperature for 12 hours at which time it was diluted with water and extracted into ethyl acetate (3×, 30 ml). The combined organic extracts were washed with brine, dried over sodium sulfate, and concentrated on the rotary evaporator. The crude product was by silica gel chromatography on the ISCO COMBI FLASH® (10% to 100% ethyl acetate in hexanes, 25 minutes). The pure fractions were pooled and concentrated to afford the intermediate as a light orange oil. Yield 68%. LC/MS [M+Na]⁺=526.0.

Step c.

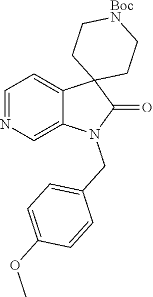 [SMILES: COc1ccc(CN2C(=O)C3(CCN(C(=O)OC(C)(C)C)CC3)c3ccncc32)cc1]

A mixture of intermediate b. (17.0 g, 33.7 mmol), described in this example, palladium(II)acetate (0.76 g, 3.4 mmol), tricyclohexyl phosphine (1.9 g, 6.7 mmol) were dissolved in dioxane (40 mL) in a sealed tube. Nitrogen was gently bubbled though the mixture for 10 minutes at which point sodium t-butoxide (4.9 g, 50.5 mmol) was added and nitrogen was bubbled through the reaction mixture for an additional 10 minutes. The tube was sealed and heated at 120° C. for 16 hours. The mixture was cooled and concentrated on the rotary evaporator. The dark, viscous product mixture was purified by silica gel chromatography on the ISCO COMBI FLASH® (0% to 10% methanol in DCM, 25 minutes). The pure fractions were pooled and concentrated to afford the intermediate as a light yellow oil. Yield 84%. LC/MS [M+H]⁺=424.2.

Step d.

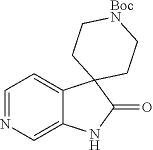 [SMILES: CC(C)(C)OC(=O)N1CCC2(CC1)C(=O)Nc1cnccc12]

The intermediate from step c of this example (3 g, 7.1 mmol) and anisole (3.8 g, 35.4 mmol) were stirred in a solution of 10% triflic acid in TFA (25 mL) at 70° C. for 12 hours (LC/MS [M+H]⁺=204.2). The mixture was cooled and concentrated on the rotary evaporator and azeotroped with toluene (3×). The dark, viscous product mixture was taken up in acetonitrile (50 mL) and cooled to 0° C. The pH was adjusted to 8 by the dropwise addition of DIPEA and boc anhydride (1.5 g, 7.1 mmoL) was added and the reaction was stirred for 40 minutes. The solvent was removed by the rotary evaporator and the crude product mixture was purified by RP HPLC (ISCO COMBI FLASH®, 10-95% acetonitrile in DI water, 0.1% TFA, 40 minute gradient). The pure fractions were pooled and lyophilized to afford the product as a white solid. Yield 69%. LC/MS [M+H]⁺=304.2.

### Synthesis of Int-12B

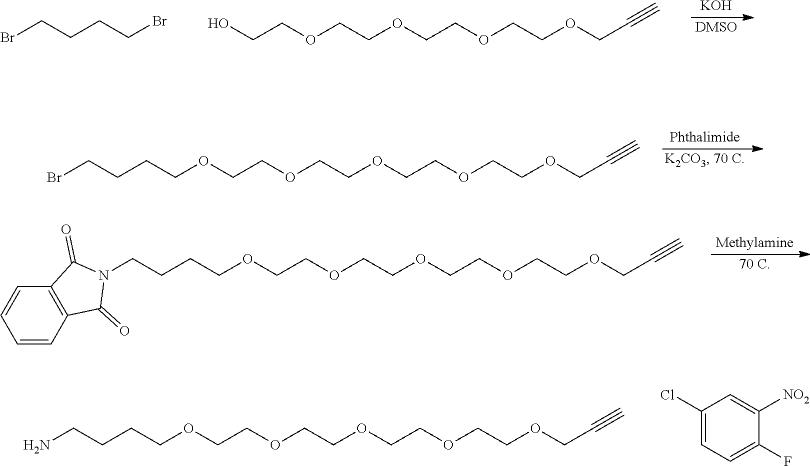

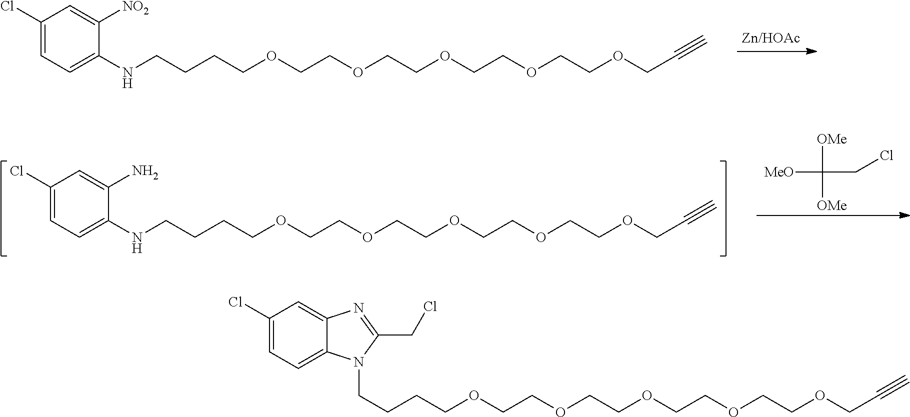

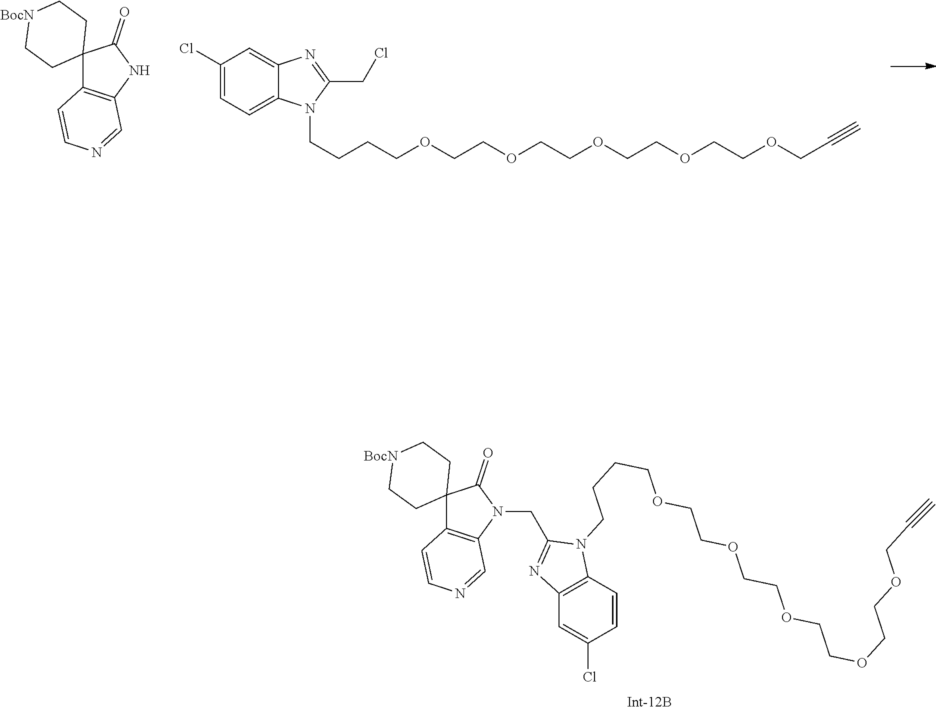 [SMILES: CC(C)(C)OC(=O)N1CCC2(CC1)C(=O)Nc1cnccc12 ; C#CCOCCOCCOCCOCCOCCCCn1c(CCl)nc2cc(Cl)ccc21 ; C#CCOCCOCCOCCOCCOCCCCn1c(CN2C(=O)C3(CCN(C(=O)OC(C)(C)C)CC3)c3ccncc32)nc2cc(Cl)ccc21]

Step a.

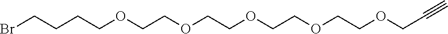 [SMILES: C#CCOCCOCCOCCOCCOCCCCBr]

A stirring solution of propargyl-PEG4-alcohol (2.00 g, 8.61 mmol) and 1,4-dibromobutane (5.57 g, 25.83 mmol), dissolved in DMSO (20 mL), at room temperature, was treated with powdered KOH (0.966 g, 17.22 mmol). The reaction initially became warm and turned dark yellow. After stirring for 1 h, LCMS shows complete consumption of alcohol. The reaction was filtered, diluted with ethylacetate, and extracted with water three times. The water washes were extracted with ethyl acetate three times. The combined ethyl acetate extracts were dried over sodium sulfate, concentrated, and purified by RPLC (10 to 100% ACN/water). Yield 1.10 g, 34.9%.

Step b.

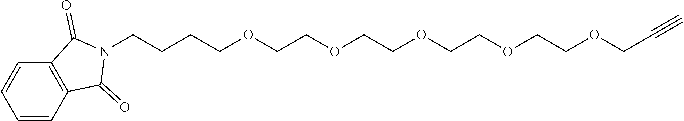 [SMILES: C#CCOCCOCCOCCOCCOCCCCN1C(=O)c2ccccc2C1=O]

To a stirring solution of product from the previous step a (1.100 g, 3.00 mmol) and phthalimide (0.881 g, 6.00 mmol) in DMF (7 mL), was added powdered potassium carbonate (1.66 g, 11.98 mmol). The mixture was stirred in a 70° C. oil bath for 1 h, at which time LCMS showed complete disappearance of starting bromide. The reaction mixture was filtered, concentrated and purified by RPLC (10 to 100% ACN/water). Yield 1.28 g, 96.6% yield. Ion(s) found by LCMS: [M+H]⁺=434.0.

Step c.

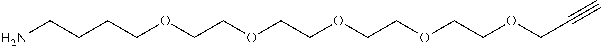 [SMILES: C#CCOCCOCCOCCOCCOCCCCN]

A solution of product from the previous step b (1.10 g, 2.54 mmol) dissolved in ethanol (3 mL), was treated with 40% aqueous methyl amine (3 mL) and heated in 70° C. oil bath for 1 h, at which time LCMS show complete consumption of starting material. The reaction was concentrated by rotary evaporation, then stored under high vacuum overnight, and used as mixture of N-methyl-phthalimide and desired product in the next step without further purification.

Step d.

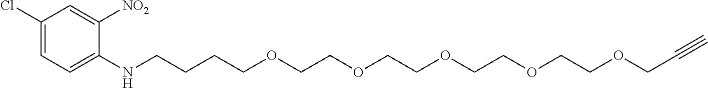 [SMILES: C#CCOCCOCCOCCOCCOCCCCNc1ccc(Cl)cc1[N+](=O)[O-]]

Crude product (2.538 mmol) from the previous step c was dissolved in DMF (5 mL), treated with DIEA (1.81 mL, 4 eq) and 5-Chloro-2-fluoronitrobenzene (0.535 g, 3.046 mmol), and heated in 50° C. oil bath. After stirring overnight LCMS showed complete consumption of amino-PEG starting material. The crude mixture was concentrated and purified by RPLC (10 to 100% ACN/water). Yield 0.62 g, 52% yield for two steps. Ion(s) found by LCMS: [M+H]⁺=459.0.

Step e.

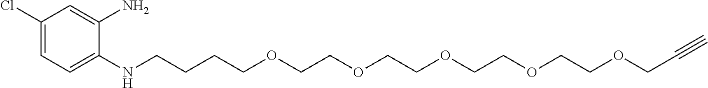 [SMILES: C#CCOCCOCCOCCOCCOCCCCNc1ccc(Cl)cc1N]

Product from the previous step d (0.620 g, 1.35 mmol), was dissolved in acetic acid (4 mL), heated in a 50° C. oil bath, and treated with zinc powder (1.77 g, 27.02 mmol), portionwise over 15 minutes. After 20 min the reaction changes color from orange to colorless, and LCMS shows complete consumption of starting material. The reaction mixture was filtered to remove zinc powder and used in the next step as a solution in acetic acid.

Step f.

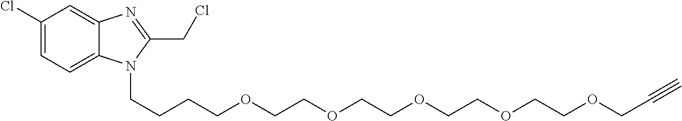 [SMILES: C#CCOCCOCCOCCOCCOCCCCn1c(CCl)nc2cc(Cl)ccc21]

Crude product from the previous step e (1.35 mmol) was heated in a 50° C. oil bath, and treated with 2-chloro-1,1,1-trimethoxyethane (1.25 g, 8.10 mmol). LCMS after 1 hr shows complete consumption of starting material. The reaction was concentrated by rotary evaporation, then purified by flash chromatography (0 to 10% MeOH/DCM). Yield 0.45 g, 68.3% yield for two steps. Ion(s) found by LCMS: [M+H]⁺=486.8.

Step g.

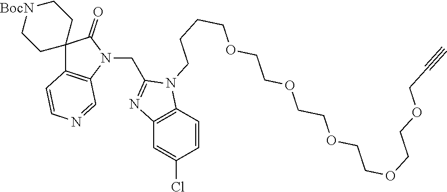 [SMILES: C#CCOCCOCCOCCOCCOCCCCn1c(CN2C(=O)C3(CCN(C(=O)OC(C)(C)C)CC3)c3ccncc32)nc2cc(Cl)ccc21]

To a solution of tert-butyl 2-oxospiro[2-pyrrolino[2,3-c]pyridine-3,4′-piperidine]-10-carboxylate (31 mg, 0.10 mmol prepared as described above) in CH₃CN (10 mL) was added Cs₂CO₃ (100 mg, 0.30 mmol). The solution was stirred for 20 min. To this was added the product from the previous step and the solution was stirred for 16 h. The excess CH₃CN was removed and the crude material was purified by reversed phase HPLC (0-100% CH₃CN/H₂O using 0.1% TFA). Ion found by LCMS: [M+H]⁺=754.2.

Click Conjugation

A solution of azido functionalized polypeptide and alkyne intermediate, Int-12B was treated with a solution (pH ˜6, adjusted with potassium hydroxide (aq)) of BTTAA (19.4 mg, 50 eq), CuSO4 (3.6 mg, 25 eq), aminoguanidine HCl (25 mg, 250 eq), zinc chloride (9.3 mg, 75 eq), and sodium ascorbate (44.8 mg, 250 eq), dissolved in 1 mL of water. Reaction progress was monitored by Maldi-TOF analysis. Yield=21.0 mg, 18.0%. MALDI-TOF=61,283. DAR=6.0.

### OTHER EMBODIMENTS

All publications, patents, and patent applications mentioned in this specification are herein incorporated by reference to the same extent as if each independent publication or patent application was specifically and individually indicated to be incorporated by reference.

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure that come within known or customary practice within the art to which the disclosure pertains and may be applied to the essential features hereinbefore set forth, and follows in the scope of the claims. Other embodiments are within the claims.

1. A method of synthesizing a conjugate of formula (M-I): 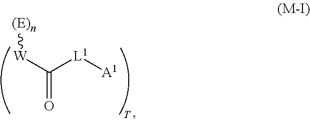
or a pharmaceutically acceptable salt thereof,
wherein
n is 1 or 2;
W is O, S, NR^{N}, or 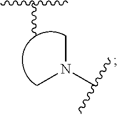
R^{N} is H, optionally substituted C₁-C₂₀ alkyl, or optionally substituted C₁-C₂₀ heteroalkyl; 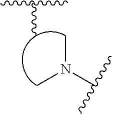
is optionally substituted C₂-C₁₀ heterocyclylene;
each E is a polypeptide or polymer;
L¹ is a linker comprising one or more of optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₂ alkynylene, optionally substituted C₂-C₂₀ heteroalkynylene, optionally substituted C₃-C₂₀ carbocyclylene, optionally substituted C₂-C₂₀ heterocyclylene, optionally substituted C₆-C₂₂ arylene, optionally substituted C₂-C₂₀ heteroarylene, carbonyl, thiocarbonyl, sulfonyl, phosphoryl, optionally substituted amino, O, and S;
each A¹ is a therapeutic agent, or each A¹ is, independently, selected from any one of H, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ carbocyclyl, optionally substituted C₂-C₉ heterocyclyl, optionally substituted O₆-C₁₀ aryl, optionally substituted C₂-C₉ heteroaryl, and optionally substituted amino;
T is an integer from 1 to 20; and
each squiggly line in formula (M-I) indicates that 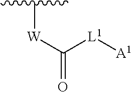
is covalently attached to each E, said method comprising:
(a) providing a first composition comprising E;
(b) providing a second composition comprising a compound of formula (F-I) or salt thereof: 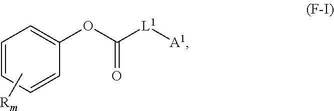
wherein
m is 0, 1, 2, 3, or 4, and
each R is, independently, halo, cyano, nitro, optionally substituted C₁-C₆ alkyl group, or optionally substituted C₁-C₆ heteroalkyl group; and
(c) combining the first composition, the second composition, and a buffer to form a mixture.

2. The method of claim 1, wherein E is a polypeptide.

3. The method of claim 2, wherein E is an Fc domain monomer, an Fc domain, an Fc-binding peptide, an albumin protein, or an albumin protein-binding peptide.

4. The method of claim 3, wherein E is an Fc domain monomer, an Fc domain, or an Fc-binding peptide.

5. The method of claim 4, wherein E is an Fc domain monomer or an Fc domain.

6. The method of any one of claims 1 to 5, wherein E comprises at least one lysine residue.

7. The method of claim 6, wherein the squiggly line in formula (M-I) is covalently bound to a lysine residue of each E.

8. The method of claim 6 or 7, wherein W is NR^{N}.

9. The method of claim 8, wherein R^{N} is H or optionally substituted C₁-C₂₀ alkyl.

10. The method of claim 9, wherein R^{N} is H.

11. The method of any one of claims 1 to 5, wherein E comprises at least one cysteine residue.

12. The method of claim 11, wherein the squiggly line in formula (M-I) is covalently bound to a cysteine residue of each E.

13. The method of claim 11 or 12, wherein W is S.

14. The method of any one of claims 1 to 5, wherein E comprises at least one proline residue.

15. The method of claim 14, wherein the squiggly line in formula (M-I) is covalently bound to a proline residue of each E.

16. The method of claim 14 or 15, wherein W is

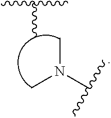

17. The method of claim 16, wherein

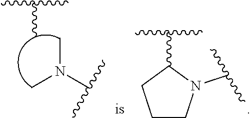

18. The method of any one of claims 1 to 17, wherein n is 1.

19. The method of any one of claims 1 to 17, wherein n is 2.

20. The method of claim 1, wherein E is a polymer.

21. The method of claim 20, wherein E comprises an amine.

22. The method of claim 21, wherein E comprises —NH₂.

23. The method of claim 22, wherein W is NH.

24. The method of any one of claims 1 to 23, wherein A¹ is a therapeutic agent.

25. The method of any one of claims 1 to 24, wherein A¹ comprises a small molecule.

26. The method of any one of claims 1 to 25, wherein L¹ is: 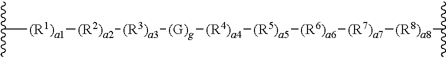
wherein
g is 0 or 1;
each of a1, a2, a3, a4, a5, a6, a7, and a8 is, independently, 0 or 1;
G is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted C₃-C₁₀ carbocyclylene, optionally substituted C₂-C₁₀ heterocyclylene, optionally substituted C₆-C₁₀ arylene, or optionally substituted C₂-C₁₀ heteroarylene;
R¹ is optionally substituted C₁-C₂₀ alkylene, optionally substituted C₁-C₂₀ heteroalkylene, optionally substituted amino, O, or S;
R² is optionally substituted C₁-C₂₀ heteroalkylene, optionally substituted C₂-C₂₀ alkenylene, optionally substituted C₂-C₂₀ heteroalkenylene, optionally substituted C₃-C₂₀ cycloalkylene, optionally substituted C₃-C₂₀ heterocycloalkylene, optionally substituted C₆-C₂₂ arylene, or optionally substituted C₂-C₂₀ heteroarylene;
R³ is optionally substituted C₁-C₂₀ alkylene, optionally substituted C₁-C₂₀ heteroalkylene, or carbonyl;
R⁴ is optionally substituted C₁-C₂₀ alkylene, optionally substituted C₁-C₂₀ heteroalkylene, or carbonyl;
R⁵ is optionally substituted C₁-C₂₀ heteroalkylene, optionally substituted C₂-C₂₀ alkenylene, optionally substituted C₂-C₂₀ heteroalkenylene, optionally substituted C₃-C₂₀ cycloalkylene, optionally substituted C₃-C₂₀ heterocycloalkylene, optionally substituted C₆-C₁₈ arylene, optionally substituted C₂-C₂₀ heteroarylene, optionally substituted amino, O, or S;
R⁶ is optionally substituted C₁-C₂₀ alkylene, optionally substituted C₁-C₂₀ heteroalkylene, or carbonyl;
R⁷ is optionally substituted C₁-C₂₀ heteroalkylene, optionally substituted C₂-C₂₀ alkenylene, optionally substituted C₂-C₂₀ heteroalkenylene, optionally substituted C₃-C₂₀ cycloalkylene, optionally substituted C₃-C₂₀ heterocycloalkylene, optionally substituted C₆-C₁₈ arylene, optionally substituted C₂-C₂₀ heteroarylene, optionally substituted amino, O, or S; and
R⁸ is optionally substituted C₁-C₂₀ alkylene, optionally substituted C₁-C₂₀ heteroalkylene, or carbonyl.

27. The method of claim 26, wherein g is 0.

28. The method of claim 26 or 27, wherein R¹ is optionally substituted C₁-C₂₀ alkylene or optionally substituted C₁-C₂₀ heteroalkylene.

29. The method of claim 28, wherein R¹ is optionally substituted C₁-C₂₀ heteroalkylene.

30. The method of claim 29, wherein R¹ is C₁-C₂₀ heteroalkylene.

31. The method of claim 30, wherein R¹ is: 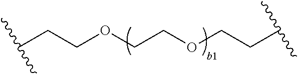
wherein b1 is 0, 1, 2, 3, 4, 5, 6, 7, or 8.

32. The method of any one of claims 26 to 31, wherein R³ is optionally substituted C₁-C₂₀ alkylene or optionally substituted C₁-C₂₀ heteroalkylene.

33. The method of claim 32, wherein R³ is optionally substituted C₁-C₂₀ heteroalkylene.

34. The method of claim 33, wherein R³ is C₁-C₂₀ heteroalkylene.

35. The method of claim 34, wherein R³ is: 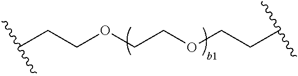
wherein b1 is 0, 1, 2, 3, 4, 5, 6, 7, or 8.

36. The method of any one of claims 26 to 35, wherein R⁴ is optionally substituted C₁-C₂₀ alkylene or optionally substituted C₁-C₂₀ heteroalkylene.

37. The method of claim 30, wherein R⁴ is: 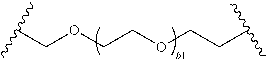
wherein b1 is 0, 1, 2, 3, 4, 5, 6, 7, or 8.

38. The method of any one of claims 26 to 37, wherein R⁵ is optionally substituted amino or optionally substituted C₃-C₂₀ heterocycloalkylene.

39. The method of any one of claims 26 to 38, wherein R⁶ is optionally substituted C₁-C₂₀ alkylene.

40. The method of any one of claims 26 to 39, wherein R⁷ is optionally substituted amino.

41. The method of any one of claims 26 to 40, wherein R⁸ is carbonyl.

42. The method of any one of claims 1 to 41, wherein each R is, independently, halo, cyano, nitro, haloalkyl, or 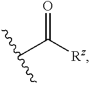
where R^{z} is optionally substituted C₁-C₅ alkyl group or optionally substituted C₁-C₅ heteroalkyl group.

43. The method of claim 42, wherein each R is, independently, halo, cyano, nitro, or haloalkyl.

44. The method of claim 43, wherein each R is, independently, F, Cl, Br, or I.

45. The method of claim 44, wherein each R is F.

46. The method of any one of claims 1 to 45, wherein m is 1, 2, 3, 4, or 5.

47. The method of claim 46, wherein m is 3 or 4.

48. The method of claim 47, wherein m is 4.

49. The method of claim 48, wherein

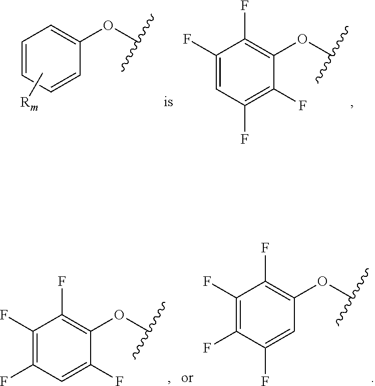

50. The method of claim 49, wherein

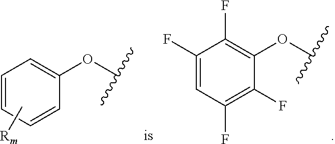

51. The method of claim 47, wherein m is 3.

52. The method of claim 51, wherein

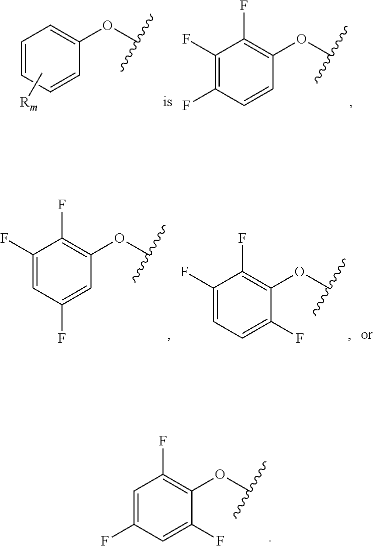

53. The method of claim 52, wherein

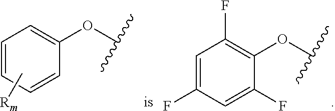

54. The method of claim 47, wherein

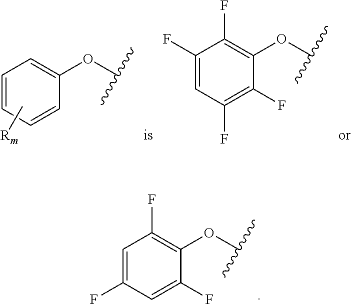

55. The method of claim 54, wherein

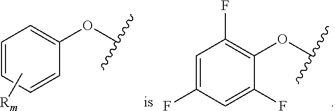

56. The method of any one of claims 1 to 55 wherein the compound of formula (F-I) is described by formula (F-I-A):

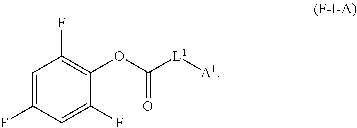

57. The method of any one of claims 1 to 55 wherein the compound of formula (F-I) is described by formula (F-I-B):

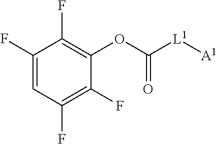

58. The method of any one of claims 1 to 57, wherein the buffer comprises borate or carbonate.

59. The method of any one of claims 1 to 58, wherein the buffer has a pH of about 7.0 to 10.0.

60. The method of claim 59, wherein the buffer has a pH of about 7.5 to 9.5.

61. The method of claim 59 or 60, wherein the buffer has a pH of about 7.5.

62. The method of claim 59 or 60, wherein the buffer has a pH of about 8.5.

63. The method of claim 59 or 60, wherein the buffer has a pH of about 9.5.

64. The method of any one of claims 1 to 63, wherein step (c) is conducted at a temperature of 20 to 30° C.

65. The method of claim 64, wherein step (c) is conducted at a temperature of 22 to 27° C.

66. The method of claim 65, wherein step (c) is conducted at a temperature of about 25° C.

67. The method of any one of claims 1 to 66, wherein step (c) is conducted for 2 to 12 hours.

68. the method of claim 66, wherein step (c) is conducted for about 2 hours.

69. The method of any one of claims 1 to 68, wherein the first composition comprises phosphate-buffered saline buffer.

70. The method of any one of claims 1 to 69, wherein the buffer has a pH of about 7.0 to 8.0.

71. The method of claim 70, wherein the buffer has a pH of about 7.5.

72. The method of any one of claims 1 to 71, wherein the second composition comprises DMF.

73. The method of any one of claims 1 to 72, wherein the method further comprises a purification step.

74. The method of claim 73, wherein the purification step comprises dialysis in arginine buffer.

75. The method of claim 73 or 74, wherein the purification step comprises a buffer exchange.

76. A method of synthesizing a conjugate of formula (M-II): 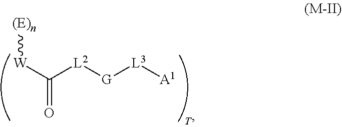
or a pharmaceutically acceptable salt thereof,
wherein
n is 1 or 2;
W is O, S, NR^{N} or 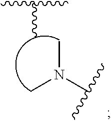
R^{N} is H, optionally substituted C₁-C₂₀ alkylene, or optionally substituted C₁-C₂₀ heteroalkylene; 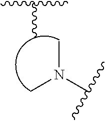
is optionally substituted C₂-C₁₀ heterocyclylene;
each E is a polypeptide or polymer;
L² is a linker comprising one or more of optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ carbocyclylene, optionally substituted C₂-C₂₀ heterocyclylene, optionally substituted C₆-C₂₂ arylene, optionally substituted C₂-C₂₀ heteroarylene, carbonyl, thiocarbonyl, sulfonyl, phosphoryl, optionally substituted amino, O, and S;
L³ is a linker comprising one or more of optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ carbocyclylene, optionally substituted C₂-C₂₀ heterocyclylene, optionally substituted C₆-C₂₂ arylene, optionally substituted C₂-C₂₀ heteroarylene, carbonyl, thiocarbonyl, sulfonyl, phosphoryl, optionally substituted amino, O, and S;
G is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted C₃-C₁₀ carbocyclylene, optionally substituted C₂-C₁₀ heterocyclylene, optionally substituted C₆-C₁₀ arylene, or optionally substituted C₂-C₁₀ heteroarylene;
each A¹ is a therapeutic agent, or each A¹ is, independently, selected from any one of H, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, and optionally substituted amino;
T is an integer from 1 to 20; and
each squiggly line in formula (M-II) indicates that 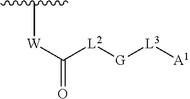
is covalently attached to each E,
said method comprising:
(a) providing a first composition comprising E;
(b) providing a second composition comprising a compound of formula (F-II) or salt thereof: 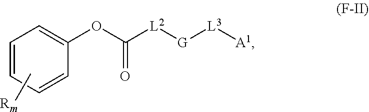
wherein
m is 0, 1, 2, 3, or 4, and
each R is, independently, halo, cyano, nitro, optionally substituted C₁-C₆ alkyl group, or optionally substituted C₁-C₆ heteroalkyl group; and
(c) combining the first composition, the second composition, and a buffer to form a mixture.

77. The method of claim 76, wherein G is optionally substituted C₁-C₆ heteroalkylene or optionally substituted C₂-C₁₀ heteroarylene.

78. The method of claim 77, wherein G is optionally substituted C₁-C₆ heteroalkylene.

79. The method of claim 78, wherein G is 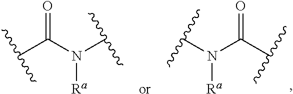
wherein R^{a} is H, optionally substituted C₁-C₂₀ alkylene, or optionally substituted C₁-C₂₀ heteroalkylene.

80. The method of claim 77, wherein G is optionally substituted C₂-C₁₀ heteroarylene.

81. The method of claim 80, wherein G is optionally substituted C₂-C₅ heteroarylene.

82. The method of claim 81, wherein G is a 5-membered or 6-membered optionally substituted C₂-C₅ heteroarylene.

83. The method of claim 82, wherein G is a triazolylene.

84. The method of claim 83, wherein the conjugate of formula (M-II) is described by the formula (M-II-A): 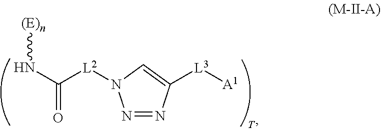
and said method comprises:
(a) providing a first composition comprising E;
(b) providing a second composition comprising a compound of formula (F-II-A) or salt thereof: 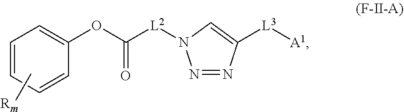
and
(c) combining the first composition, the second composition, and a buffer to form a mixture.

85. The method of claim 84, wherein the synthesis of compound of formula (F-II-A) comprises:
(d) providing a third composition comprising formula (G1-A) or salt thereof: 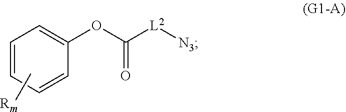
(e) providing a fourth composition comprising formula (G1-B) or salt thereof: 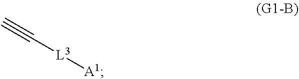
and
(f) combing the third composition and the fourth composition to form a mixture.

86. The method of claim 84, wherein the compound of formula (F-II-A) is described by formula (F-II-A-1):

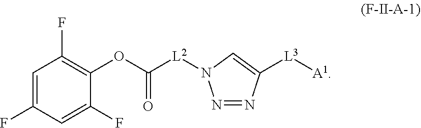

87. The method of claim 84, wherein the compound of formula (F-II-A) is described by formula (F-II-A-2):

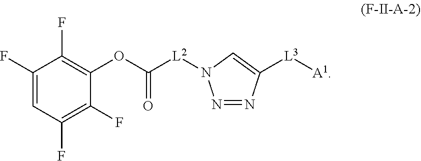

88. The method of claim 85, wherein the compound of formula (G1-A) is described by formula (G1-A-1):

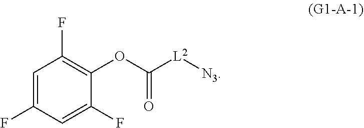

89. The method of claim 85, wherein the compound of formula (G1-A) is described by formula (G1-A-2):

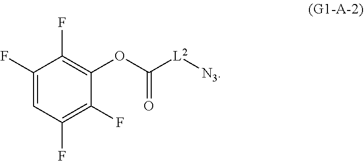

90. The method of claim 83, wherein the conjugate of formula (M-II) is described by the formula (M-II-B): 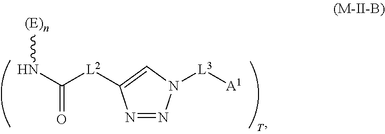
and said method comprises:
(a) providing a first composition comprising E;
(b) providing a second composition comprising a compound of formula (F-II-A) or salt thereof: 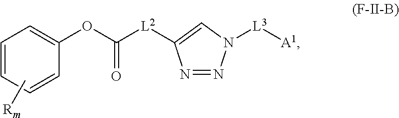
and
(c) combining the first composition, the second composition, and a buffer to form a mixture.

91. The method of claim 90, wherein the synthesis of compound of formula (F-II-B) comprises:
(d) providing a third composition comprising formula (G2-A) or salt thereof: 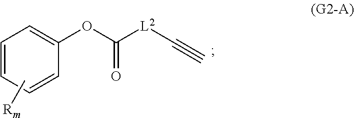
(e) providing a fourth composition comprising formula (G2-B) or salt thereof: 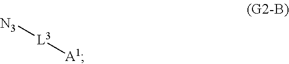
and
(f) combing the third composition and the fourth composition to form a mixture.

92. The method of claim 90, wherein the compound of formula (F-II-B) is described by formula (F-II-B-1).

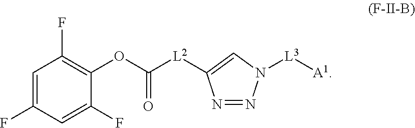

93. The method of claim 90, wherein the compound of formula (F-II-B) is described by formula (F-II-B-2):

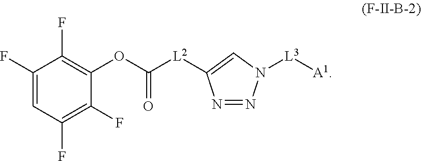

94. The method of claim 91, wherein the compound of formula (G2-A) is described by formula (G2-A-1):

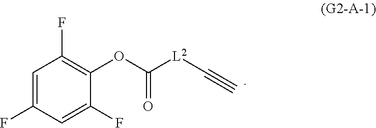

95. The method of claim 91, wherein the compound of formula (G2-A) is described by formula (G2-A-2):

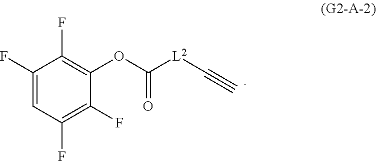

96. The method of claim 85 or 91, wherein step (f) comprises the use of a Cu(I) source.

97. A method of synthesizing a conjugate of formula (M-II): 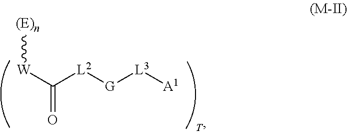
or a pharmaceutically acceptable salt thereof,
wherein
n is 1 or 2;
W is O, S, NR^{N}, or 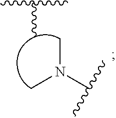
R^{N} is H, optionally substituted C₁-C₂₀ alkylene, or optionally substituted C₁-C₂₀ heteroalkylene; 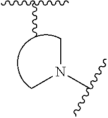
is optionally substituted C₂-C₁₀ heterocyclylene;
each E is a polypeptide or polymer;
L² is a linker comprising one or more of optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ carbocyclylene, optionally substituted C₂-C₂₀ heterocyclylene, optionally substituted C₆-C₂₂ arylene, optionally substituted C₂-C₂₀ heteroarylene, carbonyl, thiocarbonyl, sulfonyl, phosphoryl, optionally substituted amino, O, and S;
L³ is a linker comprising one or more of optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ carbocyclylene, optionally substituted C₂-C₂₀ heterocyclylene, optionally substituted C₆-C₂₂ arylene, optionally substituted C₂-C₂₀ heteroarylene, carbonyl, thiocarbonyl, sulfonyl, phosphoryl, optionally substituted amino, O, and S;
G is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted C₃-C₁₀ carbocyclylene, optionally substituted C₂-C₁₀ heterocyclylene, optionally substituted C₆-C₁₀ arylene, or optionally substituted C₂-C₁₀ heteroarylene;
each A¹ is a therapeutic agent, or each A¹ is, independently, selected from any one of H, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, and optionally substituted amino;
T is an integer from 1 to 20; and
each squiggly line in formula (M-II) indicates that 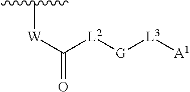
is covalently attached to each E,
said method comprising:
(a) providing a first composition comprising formula (G3-A) or a salt thereof: 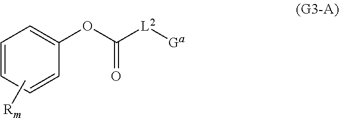
wherein G^{a} is a functional group that reacts with G^{b} to form G;
(b) providing a second composition comprising formula (G3-B) or a salt thereof: 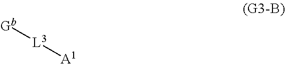
wherein G^{b} is a functional group that reacts with G^{a} to form G; and
(c) combining the first composition and the second composition to form a first mixture,
wherein
m is 0, 1, 2, 3, or 4; and
each R is, independently, halo, cyano, nitro, optionally substituted C₁-C₆ alkyl group, or optionally substituted C₁-C₆ heteroalkyl group.

98. The method of claim 97, wherein step (c) comprises the use of a Cu(I) source.

99. The method of claim 97 or 98, wherein the method further comprises:
(d) providing a third composition comprising E; and
(e) combing the third composition, the first mixture, and a buffer to form a second mixture.

100. The method of any one of claims 97 to 99, wherein G^{a} comprises optionally substituted amino.

101. The method of claim 100, wherein G^{b} comprises a carbonyl.

102. The method of any one of claims 97 to 99, wherein G^{a} comprises a carbonyl.

103. The method of claim 102, wherein G^{b} comprises optionally substituted amino.

104. The method of any one of claims 97 to 99, wherein G^{a} comprises an azido group.

105. The method of claim 104, wherein G^{b} comprises an alkynyl group.

106. The method of any one of claims 97 to 99, wherein G^{a} comprises an alkynyl group.

107. The method of claim 106, wherein G^{b} comprises an azido group.

108. The method of claim 97, wherein the compound of formula (G3-A) is described by formula (G3-A-1):

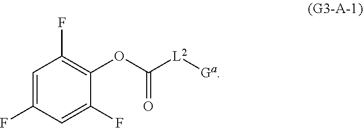

109. The method of claim 97, wherein the compound of formula (G3-A) is described by formula (G3-A-2):

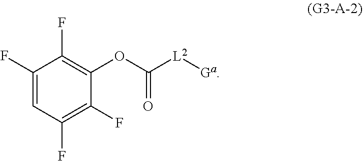

110. The method of any one of claims 76 to 109, wherein E is a polypeptide.

111. The method of claim 110, wherein E is an Fc domain monomer, an Fc domain, an albumin protein, an albumin protein-binding peptide, or an Fc-binding peptide.

112. The method of claim 111, wherein E is an Fc domain monomer, an Fc domain, or an Fc-binding peptide.

113. The method of claim 112, wherein E is an Fc domain monomer or an Fc domain.

114. The method of any one of claims 76 to 113, wherein E comprises at least one lysine residue.

115. The method of claim 114, wherein the squiggly line in formula (M-I) is covalently bound to a lysine residue of each E.

116. The method of claim 114 or 115, wherein W is NR^{N}.

117. The method of claim 116, wherein R^{N} is H or optionally substituted C₁-C₂₀ alkyl.

118. The method of claim 117, wherein R^{N} is H.

119. The method of any one of claims 76 to 113, wherein E comprises at least one cysteine residue.

120. The method of claim 119, wherein the squiggly line in formula (M-II) is covalently bound to a cysteine residue of each E.

121. The method of claim 119 or 120, wherein W is S.

122. The method of any one of claims 76 to 113, wherein E comprises at least one proline residue.

123. The method of claim 122, wherein the squiggly line in formula (M-I) is covalently bound to a proline residue of each E.

124. The method of claim 122 or 123, wherein W is.

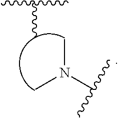

125. The method of claim 124,

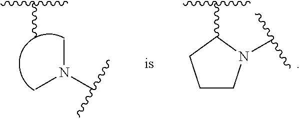

126. The method of any one of claims 76 to 125, wherein n is 1.

127. The method of any one of claims 76 to 125, wherein n is 2.

128. The method of any one of claims 76 to 109, wherein E is a polymer.

129. The method of claim 128, wherein E comprises an amine.

130. The method of claim 129, wherein E comprises —NH₂.

131. The method of claim 130, wherein W is NH.

132. The method of any one of claims 76 to 131, wherein A¹ is a therapeutic agent.

133. The method of any one of claims 76 to 132, wherein A¹ includes a small molecule.

134. The method of any one of claims 76 to 133, wherein L² is: 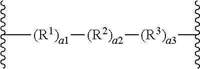
wherein
each of a1, a2, and a3 is, independently, 0 or 1;
R¹ is optionally substituted C₁-C₂₀ alkylene, optionally substituted C₁-C₂₀ heteroalkylene, optionally substituted amino, O, or S;
R² is optionally substituted C₁-C₂₀ heteroalkylene, optionally substituted C₂-C₂₀ alkenylene, optionally substituted C₂-C₂₀ heteroalkenylene, optionally substituted C₃-C₂₀ cycloalkylene, optionally substituted C₃-C₂₀ heterocycloalkylene, optionally substituted C₆-C₁₈ arylene, or optionally substituted C₂-C₂₀ heteroarylene; and
R³ is optionally substituted C₁-C₂₀ alkylene, optionally substituted C₁-C₂₀ heteroalkylene, or carbonyl.

135. The method of claim 134, wherein a2 is 0.

136. The method of claim 134 or 135, wherein a1 is 1 and a3 is 0.

137. The method of claim 134 or 135, wherein a1 is 1 and a3 is 1.

138. The method of any one of claims 134 to 137, wherein R¹ is optionally substituted C₁-C₂₀ alkylene or optionally substituted C₁-C₂₀ heteroalkylene.

139. The method of claim 138, wherein R¹ is optionally substituted C₁-C₂₀ heteroalkylene.

140. The method of claim 139, wherein R¹ is: 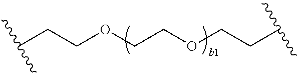
wherein b1 is 0, 1, 2, 3, 4, 5, 6, 7, or 8.

141. The method of any one of claims 134 to 140, wherein R³ is optionally substituted C₁-C₂₀ alkylene or optionally substituted C₁-C₂₀ heteroalkylene.

142. The method of claim 141, wherein R³ is optionally substituted C₁-C₂₀ heteroalkylene.

143. The method of claim 142, wherein R³ is: 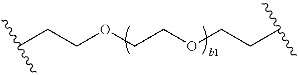
wherein b1 is 0, 1, 2, 3, 4, 5, 6, 7, or 8.

144. The method of any one of claims 76 to 143, wherein L³ is: 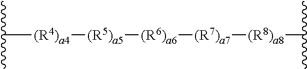
wherein
each of a4, a5, a6, a7, and a8 is, independently, 0 or 1;
R⁴ is optionally substituted C₁-C₂₀ alkylene, optionally substituted C₁-C₂₀ heteroalkylene, or carbonyl;
R⁵ is optionally substituted C₁-C₂₀ heteroalkylene, optionally substituted C₂-C₂₀ alkenylene, optionally substituted C₂-C₂₀ heteroalkenylene, optionally substituted C₃-C₂₀ cycloalkylene, optionally substituted C₃-C₂₀ heterocycloalkylene, optionally substituted C₆-C₁₈ arylene, optionally substituted C₂-C₂₀ heteroarylene, optionally substituted amino, O, or S;
R⁶ is optionally substituted C₁-C₂₀ alkylene, optionally substituted C₁-C₂₀ heteroalkylene, or carbonyl;
R⁷ is optionally substituted C₁-C₂₀ heteroalkylene, optionally substituted C₂-C₂₀ alkenylene, optionally substituted C₂-C₂₀ heteroalkenylene, optionally substituted C₃-C₂₀ cycloalkylene, optionally substituted C₃-C₂₀ heterocycloalkylene, optionally substituted C₆-C₁₈ arylene, optionally substituted C₂-C₂₀ heteroarylene, optionally substituted amino, O, or S; and
R⁸ is optionally substituted C₁-C₂₀ alkylene, optionally substituted C₁-C₂₀ heteroalkylene, or carbonyl.

145. The method of claim 144, wherein a4 is 1, a5 is 1, a6 is 1, a7 is 1, and a8 is 1.

146. The method of claim 144 or 145, wherein R⁴ is optionally substituted C₁-C₂₀ alkylene or optionally substituted C₁-C₂₀ heteroalkylene.

147. The method of claim 146, wherein R⁴ is: 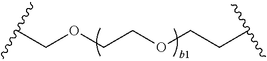
wherein b1 is 0, 1, 2, 3, 4, 5, 6, 7, or 8.

148. The method of any one of claims 144 to 147, wherein R⁵ is optionally substituted amino or optionally substituted C₃-C₂₀ heterocycloalkylene.

149. The method of any one of claims 144 to 148, wherein R⁶ is optionally substituted C₁-C₂₀ alkylene.

150. The method of any one of claims 144 to 149, wherein R⁷ is optionally substituted amino.

151. The method of any one of claims 144 to 150, wherein R⁸ is carbonyl.

152. The method of any one of claims 76 to 151, wherein each R is, independently, halo, cyano, nitro, haloalkyl, or 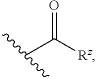
where R^{z} is optionally substituted C₁-C₅ alkyl group or optionally substituted C₁-C₅ heteroalkyl group.

153. The method of claim 152, wherein each R is, independently, halo, cyano, nitro, or haloalkyl.

154. The method of claim 153, wherein each R is, independently, F, Cl, Br, or I.

155. The method of claim 154, wherein each R is F.

156. The method of any one of claims 76 to 155, wherein m is 1, 2, 3, 4, or 5.

157. The method of claim 156, wherein m is 3 or 4.

158. The method of claim 157, wherein m is 4.

159. The method of claim 158, wherein

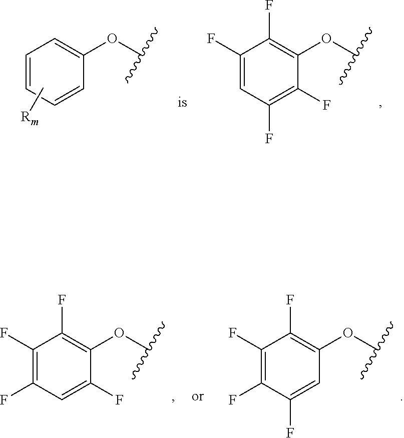

160. The method of claim 159, wherein

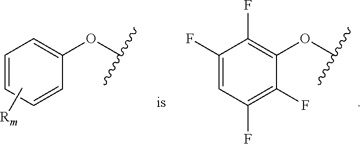

161. The method of claim 157, wherein m is 3.

162. The method of claim 161, wherein

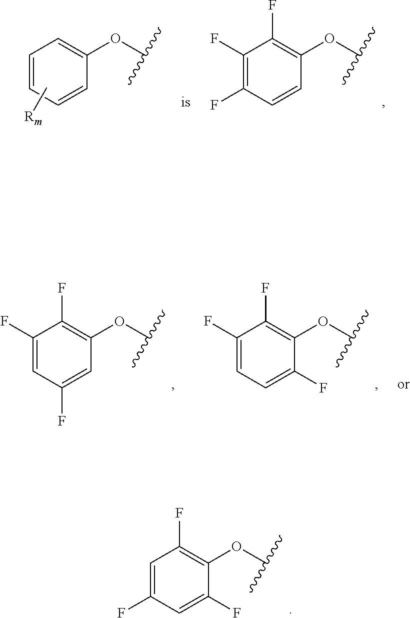

163. The method of claim 162, wherein

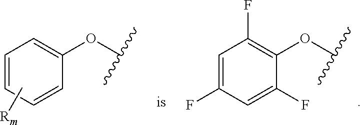

164. The method of claim 163, wherein

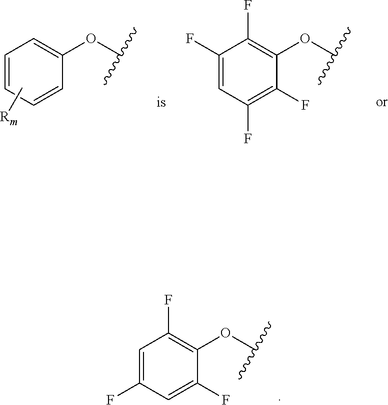

165. The method of claim 164, wherein

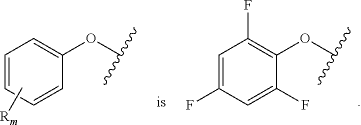

166. The method of any one of claims 76 to 165, wherein the buffer comprises borate or carbonate.

167. The method of any one of claims 76 to 166, wherein the buffer has a pH of about 7.0 to 10.0.

168. The method of claim 167, wherein the buffer has a pH of about 7.5 to 9.5.

169. The method of claim 167 or 168, wherein the buffer has a pH of about 7.5.

170. The method of claim 167 or 168, wherein the buffer has a pH of about 8.5.

171. The method of claim 167 or 168, wherein the buffer has a pH of about 9.5.

172. The method of any one of claims 76 to 171, wherein step (c) is conducted at a temperature of 20 to 30° C.

173. The method of claim 172, wherein step (c) is conducted at a temperature of 22 to 27° C.

174. The method of claim 173, wherein step (c) is conducted at a temperature of about 25° C.

175. The method of any one of claims 76 to 174, wherein step (c) is conducted for 2 to 12 hours.

176. the method of claim 175, wherein step (c) is conducted for about 2 hours.

177. The method of any one of claims 76 to 176, wherein the first composition comprises phosphate-buffered saline buffer.

178. The method of any one of claims 76 to 177, wherein the buffer has a pH of about 7.0 to 8.0.

179. The method of claim 178, wherein the buffer has a pH of about 7.5.

180. The method of any one of claims 76 to 179, wherein the second composition comprises DMF.

181. The method of any one of claims 76 to 180, wherein the method further comprises a purification step.

182. The method of claim 181, wherein the purification step comprises dialysis in arginine buffer.

183. The method of claim 181 or 182, wherein purification step comprises a buffer exchange.

184. A conjugate produced by the method of any one of claims 1 to 183.

185. The method or conjugate of any one of claims 1 to 184, wherein T is 1.

186. The method or conjugate of any one of claims 1 to 184, wherein T is 2 to 20.

187. The method or conjugate of any one of claims 1 to 184, wherein T is 2 to 10.

188. The method or conjugate of any one of claims 1 to 184, wherein T is 2 to 5.

189. A population of conjugates produced by the method of any one of claims 1 to 183.

190. The population of conjugates of claim 189, wherein the average value of T is about 1.

191. The population of conjugates of claim 189, wherein the average value of T is 2 to 20.

192. The population of conjugates of claim 189, wherein the average value of T is 2 to 10.

193. The population of conjugates of claim 189, wherein the average value of T is 2 to 5.


SEQUENCE LISTING (5 sequences)

SEQ ID NO: 1 - protein, 247 aa, Artificial Sequence
  Synthetic Construct
  MISC_FEATURE (1)..(1): Xaa is Asn or absent
  MISC_FEATURE (20)..(20): Xaa is Cys or Ser
  MISC_FEATURE (46)..(46): Xaa is Lys, Ser, Gly, Ala, Thr, Asn, Gln, Arg, His, Glu, or Asp
  MISC_FEATURE (52)..(52): Xaa is Met or Tyr
  MISC_FEATURE (54)..(54): Xaa is Ser or Thr
  MISC_FEATURE (56)..(56): Xaa is Thr or Glu
  MISC_FEATURE (97)..(97): Xaa is Asn or Ala
  MISC_FEATURE (109)..(109): Xaa is Leu or Asp
  MISC_FEATURE (111)..(111): Xaa is Gln or His
  MISC_FEATURE (156)..(156): Xaa is Asp or Glu
  MISC_FEATURE (158)..(158): Xaa is Leu or Met
  MISC_FEATURE (228)..(228): Xaa is Met or Leu
  MISC_FEATURE (234)..(234): Xaa is Asn or Ser
  MISC_FEATURE (247)..(247): Xaa is Lys or absent
XVNHKPSNTKVDKKVEPKSXDKTHTCPPCPAPELLGGPSVFLFPPXPKDTLXIXRXPEVTCVVVDVSHEDPEVKFNWYVDGVEVHNAKTKPREEQYXSTYRVVSVLTVXHXDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGX

SEQ ID NO: 2 - protein, 246 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 246 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 246 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 246 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG